(19) United States
(12) Patent Application Publication
(10) Pub. No.: US 2026/0137714 A1
(43) Pub. Date: May 21, 2026

(54) METHODS AND COMPOSITIONS FOR TREATMENT OF OPHTHALMIC DISEASE

(71) Applicant: Design Therapeutics, Inc., Carlsbad, CA (US)

(72) Inventors: Pratik SHAH, Carlsbad, CA (US); Sean J. JEFFRIES, Carlsbad, CA (US)

(21) Appl. No.: 19/122,445

(22) PCT Filed: Oct. 19, 2023
(86) PCT No.: PCT/US2023/035544

§ 371 Date: Apr. 18, 2025

Related U.S. Application Data
(60) Provisional application No. 63/380,332, filed on Oct. 20, 2022.

(51) Int. Cl. A61K 31/787; A61K 9/00; A61P 27/02

(52) U.S. Cl. CPC A61K 31/787; A61K 9/0048; A61P 27/02

(57) ABSTRACT

Provided herein are compositions for ophthalmic administration and methods of use.

### CROSS REFERENCE

This application claims the benefit of U.S. Application No. 63/380,332, filed Oct. 20, 2022, which is hereby incorporated by reference in its entirety.

### BACKGROUND

Drug delivery to the human eye is an important area of pharmaceutical development. The anatomy and physiology of the eye creates challenges in getting drugs to the site of action and maintaining therapeutic potency over time.

### SUMMARY

In an aspect, provided herein are compositions comprising a double stranded deoxyribonucleic acid (DNA) binding moiety that binds to double stranded DNA in a sequence specific manner. In some embodiments, the double stranded DNA binding moiety does not bind to a repeat sequence comprising multiple copies of GAA. In some embodiments, the composition is suitable for administration to a human eye.

In another aspect, provided herein are compositions comprising a double stranded DNA binding moiety suitable for treating a genetic disease, wherein the composition is suitable for administration to a human eye.

In a further aspect, provided herein are compositions comprising a double stranded DNA binding moiety that binds to a repeat sequence comprising at least four nucleotides, wherein the composition is suitable for administration to a human eye.

A composition comprising a double stranded DNA binding moiety that binds to a repeat sequence comprising multiple copies of CTG, TGC, CAG, GGC, TCCAA, GGGGCC, CGG, GCC, GCG, ATTCT, TGGAA, GGCCTG, CCCCGCCCCGCG, or CCTG, wherein the composition is suitable for administration to a human eye.

A composition comprising a double stranded DNA binding moiety, wherein the double stranded DNA binding moiety is a polymer that does not comprise nucleotides or amino acids, and wherein the composition is suitable for administration to a human eye.

In various aspects of compositions provided herein, in some embodiments, the double stranded DNA binding moiety binds to a repeat sequence comprising multiple copies of CTG or CAG. In some embodiments, the double stranded DNA binding moiety binds to a minor groove of double stranded DNA. In some embodiments, the double stranded DNA binding moiety binds to a repeat sequence that is adjacent to or within a gene. In some embodiments, the double stranded DNA binding moiety modulates expression of the gene. In some embodiments, the gene is Transcription factor 4 (TCF4). In some embodiments, the double stranded DNA binding moiety binds non-covalently. In some embodiments, the double stranded DNA binding moiety does not comprise a polynucleotide. In some embodiments, the double stranded DNA binding moiety does not comprise a polypeptide. In some embodiments, the double stranded DNA binding moiety has a molecular weight of less than about 10 kDa, about 7.5 kDa, about 5 kDa, or about 2.5 kDa. In some embodiments, the double stranded DNA binding moiety comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker. In some embodiments, administration of the composition to an eye results in minimal systemic exposure. In some embodiments, the composition further comprises an excipient. In some embodiments, the ophthalmic formulation composition has a pH of about 5 to about 8. In some embodiments, the composition has a viscosity of from about 1 to about 50,000 cps at about 20° C.

In another aspect, provided herein are methods of treating an eye disorder in a human individual in need thereof, the method comprising administering a double stranded deoxyribonucleic acid (DNA) binding moiety that binds to double stranded DNA in a sequence specific manner. In some embodiments, the double stranded DNA binding moiety does not bind to a repeat sequence comprising multiple copies of GAA.

In another aspect, provided herein are methods of treating an eye disorder in a human individual in need thereof, the method comprising administering a double stranded DNA binding moiety, wherein the eye disorder is a genetic disease.

In a further aspect, provided herein are methods of treating an eye disorder in a human individual in need thereof, the method comprising administering a double stranded DNA binding moiety to an eye of the individual, wherein administration results in minimal systemic exposure to the double stranded DNA binding moiety, and wherein the double stranded DNA binding moiety binds to a repeat sequence comprising multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG.

In another aspect, provided herein are methods of treating an eye disorder in a human individual in need thereof, the method comprising administering a double stranded DNA binding moiety that binds to a repeat sequence comprising at least four nucleotides.

In a further aspect, provided herein are methods of treating an eye disorder in a human individual in need thereof, the method comprising administering a double stranded DNA binding moiety, wherein the double stranded DNA binding moiety is a polymer that does not comprise nucleotides or amino acids.

In various aspects of methods herein, in some embodiments, the double stranded DNA binding moiety binds to a repeat sequence comprising multiple copies of CTG or CAG. In some embodiments, the double stranded DNA binding moiety binds to a minor groove of double stranded DNA. In some embodiments, the double stranded DNA binding moiety binds to a repeat sequence adjacent to or within a gene. In some embodiments, the double stranded DNA binding moiety modulates expression of the gene. In some embodiments, the gene is Transcription factor 4 (TCF4). In some embodiments, the double stranded DNA binding moiety binds non-covalently. In some embodiments, the double stranded DNA binding moiety does not comprise a polynucleotide. In some embodiments, the double stranded DNA binding moiety does not comprise a polypeptide. In some embodiments, the double stranded DNA binding moiety has a molecular weight of less than about 10 kDa, about 7.5 kDa, about 5 kDa, or about 2.5 kDa. In some embodiments, the double stranded DNA binding moiety comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker. In some embodiments, the double stranded DNA binding moiety is formulated in an excipient suitable for administration to the eye. In some embodiments, the double stranded DNA binding moiety is in a formulation having a pH of about 5 to about 8. In some embodiments, the double stranded DNA binding moiety is in a formulation having a viscosity of from about 1 to about 50,000 cps at about 20° C.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows ex vivo bovine eye distribution of compound 201.

### DETAILED DESCRIPTION

In treatment of diseases affecting the eyes, treatment of the affected cells and tissues of the eye is needed, while systemic administration and exposure is not necessary or desired. Therefore, in many cases, it is beneficial to administer treatments for eye diseases directly to the eye and minimize exposure to the rest of the body. Such characteristics of eye disease treatments, in some cases, avoid off target effects, reduce immune responses to the pharmaceutical compositions, and increase therapeutic efficacy by ensuring that the maximum dose reaches the target cells and tissues of the eye.

In particular, some diseases of the eye are treated with compositions comprising agents that bind to double stranded deoxyribonucleic acid (DNA), further described elsewhere herein. Administering such compositions directly to the eye with minimal systemic exposure has not been previously demonstrated, and thus the art has not demonstrated ophthalmic administration of these compositions.

Provided herein are compositions for administration to the eye, such as an ophthalmic composition comprising agents formulated for administration to the eye, and methods of treatment of eye diseases using such compositions. In some embodiments, there are provided compositions comprising agents that act by modulating gene expression by binding to double stranded deoxyribonucleic acid (DNA). In some embodiments, such compositions comprise agents that modulate genes adjacent to a particular DNA sequence by binding to that sequence and recruiting transcriptional modulators such as transcriptional activators or repressors thereby modulating expression of one or more genes. In additional embodiments, administration of such compositions to the eye results in the therapeutic staying in the eye and providing minimal exposure to the rest of the body.

### Double-Stranded Deoxyribonucleic Acid Binding Compositions

Provided herein are compositions comprising a double stranded deoxyribonucleic acid (DNA) binding moiety. In some embodiments, the double stranded DNA binding moiety binds to a repeat sequence. In some embodiments, the repeat sequence does not comprise multiple copies of GAA. In some embodiments, the repeat sequence comprises a repeat of at least four nucleotides. In some embodiments, the repeat sequence comprises multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG. In some embodiments, the repeat sequence comprises multiple copies of CTG or CAG. In some embodiments, the repeat sequence is not a triplet repeat. In some embodiments, the double stranded DNA binding moiety is suitable for treating a genetic disease. In some embodiments, the genetic disease is not Friedreich's ataxia. In some embodiments, the double stranded DNA binding moiety is a polymer that does not comprise nucleotides or amino acids. In some embodiments, the double stranded DNA binding moiety is not an antibiotic. In some embodiments, the composition is suitable for administration to a human eye. In some embodiments, the double stranded DNA binding moiety binds to a minor groove of the double stranded DNA.

In aspects of compositions provided herein, the double stranded DNA binding moiety binds to a sequence, such as a repeat sequence, that is adjacent to or within a gene. In some embodiments, the double stranded DNA binding moiety modulates expression of the gene. In some embodiments the double stranded DNA binding moiety increases expression of the gene. In some embodiments, the double stranded DNA binding moiety decreases expression of the gene. In some embodiments, the gene is Transcription factor 4 (TCF4).

In aspects of compositions provided herein, the double stranded DNA binding moiety binds to double stranded DNA non-covalently. In some embodiments, the double stranded DNA binding moiety binds to double stranded DNA using electrostatic forces, such as hydrogen bonds, or Van der Waals forces. In some embodiments, the double stranded DNA binding moiety does not comprise a polynucleotide. In some embodiments, the double stranded DNA binding moiety does not comprise a polypeptide.

In aspects of compositions provided herein, the double stranded DNA binding moiety has a molecular weight of less than about 15 kDa, less than about 12.5 kDa, less than about 10 kDa, less than about 9.5 kDa, less than about 9 kDa, less than about 8.5 kDa, less than about 8 kDa, less than about 7.5 kDa, less than about 7 kDa, less than about 6.5 kDa, less than about 6 kDa, less than about 5.5 kDa, less than about 5 kDa, less than about 4.5 kDa, less than about 4 kDa, less than about 3.5 kDa, less than about 3 kDa, less than about 2.5 kDa, less than about 2 kDa, or less than about 1.5 kDa. In some embodiments, the double stranded DNA binding moiety has a molecular weight of less than about 10 kDa, about 7.5 kDa, about 5 kDa, or about 2.5 kDa.

In some embodiments, the double stranded DNA binding moiety comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker.

In various aspects, the compositions are formulated to be suitable for topical administration. In some embodiments, the composition further comprises an excipient. In some embodiments, the excipient comprises an isotonicity adjusting agent such as sodium chloride, a buffer, a stabilizer, an anti-oxidant, a viscosity enhancing agent, a solubilizer, a penetration enhancer, and/or a preservative. In some embodiments, the composition has a pH of about 5 to about 8. In some embodiments, the composition has a viscosity of from about 1 to about 50,000 centipoise (cps) at about 20° C.

In various aspects, administration of the composition to an eye results in minimal systemic exposure. In some embodiments, topical administration (e.g., ophthalmic administration) of the composition to an eye results in penetration of the double stranded DNA binding moiety to the internal cells and tissues of the eye. In some embodiments, at least about 10%, at least about 20%, at least about 30%, at least about 40%, at least about 50%, at least about 60%, at least about 70%, at least about 80%, at least about 85%, at least about 90%, at least about 95%, or at least about 99% of the ophthalmically administered double stranded DNA binding moiety penetrates the internal cells and tissues of the eye.

In another aspect, provided herein are compositions comprising a double stranded DNA binding moiety, wherein the double stranded DNA binding moiety is a polymer that does not comprise nucleotides or amino acids, and wherein the composition is suitable for administration to a human eye.

In some embodiments of compositions that do not comprise nucleotides or amino acids, the double stranded DNA binding moiety binds to a repeat sequence. In some embodiments, the repeat sequence comprises multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG. In some embodiments, the repeat sequence comprises multiple copies of CTG or CAG. In some embodiments, the repeat sequence is not a triplet repeat. In some embodiments, the repeat sequence comprises a repeat of at least four nucleotides. In some embodiments, the repeat sequence does not comprise GAA. In some embodiments, the double stranded DNA binding moiety binds to a minor groove of the double stranded DNA.

In aspects of compositions that do not comprise nucleotides or amino acids provided herein, the double stranded DNA binding moiety is suitable for treating a genetic disease. In some embodiments, the genetic disease is not Friedreich's ataxia. In some embodiments, the double stranded DNA binding moiety is not an antibiotic.

In aspects of compositions that do not comprise nucleotides or amino acids provided herein, the double stranded DNA binding moiety binds to a sequence, such as a repeat sequence, that is adjacent to or within a gene. In some embodiments, the double stranded DNA binding moiety modulates expression of the gene. In some embodiments the double stranded DNA binding moiety increases expression of the gene. In some embodiments, the double stranded DNA binding moiety decreases expression of the gene. In some embodiments, the gene is Transcription factor 4 (TCF4).

In aspects of compositions that do not comprise nucleotides or amino acids provided herein, the double stranded DNA binding moiety binds to double stranded DNA non-covalently. In some embodiments, the double stranded DNA binding moiety binds to double stranded DNA using electrostatic forces, such as hydrogen bonds, or Van der Waals forces.

In aspects of compositions that do not comprise nucleotides or amino acids provided herein, the double stranded DNA binding moiety has a molecular weight of less than about 15 kDa, less than about 12.5 kDa, less than about 10 kDa, less than about 9.5 kDa, less than about 9 kDa, less than about 8.5 kDa, less than about 8 kDa, less than about 7.5 kDa, less than about 7 kDa, less than about 6.5 kDa, less than about 6 kDa, less than about 5.5 kDa, less than about 5 kDa, less than about 4.5 kDa, less than about 4 kDa, less than about 3.5 kDa, less than about 3 kDa, less than about 2.5 kDa, less than about 2 kDa, or less than about 1.5 kDa. In some embodiments, the double stranded DNA binding moiety has a molecular weight of less than about 10 kDa, about 7.5 kDa, about 5 kDa, or about 2.5 kDa.

In some embodiments of compositions that do not comprise nucleotides or amino acids, the double stranded DNA binding moiety comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker.

In various aspects, the composition that does not comprise nucleotides or amino acids is suitable for topical administration. In some embodiments, the composition further comprises an excipient. In some embodiments, the excipient comprises an isotonicity adjusting agent such as sodium chloride, a buffer, a stabilizer, and anti-oxidant, a viscosity enhancing agent, a solubilizer, a penetration enhancer, and/or a preservative. In some embodiments, the composition has a pH of about 5 to about 8. In some embodiments, the composition has a viscosity of from about 1 to about 50,000 cps at about 20° C.

In various aspects, administration of the composition that does not comprise nucleotides or amino acids to an eye results in minimal systemic exposure. In some embodiments, topical administration (e.g., ophthalmic administration) of the composition to an eye results in penetration of the double stranded DNA binding moiety to the internal cells and tissues of the eye. In some embodiments, at least about 10%, at least about 20%, at least about 30%, at least about 40%, at least about 50%, at least about 60%, at least about 70%, at least about 80%, at least about 85%, at least about 90%, at least about 95%, or at least about 99% of the ophthalmic administered double stranded DNA binding moiety penetrates the internal cells and tissues of the eye.

In a further aspect, there are provided compositions comprising a double stranded DNA binding moiety suitable for treating a genetic disease. In some embodiments, the genetic disease is not Friedreich's ataxia. In some embodiments, the double stranded DNA binding moiety is not an antibiotic.

In some embodiments of compositions suitable for treating a genetic disease, the composition comprises a double stranded DNA binding moiety binds to a repeat sequence. In some embodiments, the repeat sequence does not comprise multiple copies of GAA. In some embodiments, the repeat sequence comprises multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG. In some embodiments, the repeat sequence comprises multiple copies of CTG or CAG. In some embodiments, the repeat sequence comprises a repeat of at least four nucleotides. In some embodiments, the double stranded DNA binding moiety binds to a minor groove of the double stranded DNA.

In aspects of compositions suitable for treating a genetic disease provided herein, the double stranded DNA binding moiety binds to a sequence that is adjacent to or within a gene. In some embodiments, the double stranded DNA binding moiety modulates expression of the gene. In some embodiments the double stranded DNA binding moiety increases expression of the gene. In some embodiments, the double stranded DNA binding moiety decreases expression of the gene. In some embodiments, the gene is Transcription factor 4 (TCF4).

In aspects of compositions suitable for treating genetic diseases provided herein, the double stranded DNA binding moiety binds to double stranded DNA non-covalently. In some embodiments, the double stranded DNA binding moiety binds to double stranded DNA using electrostatic forces, such as hydrogen bonds, or Van der Waals forces. In some embodiments, the double stranded DNA binding moiety does not comprise a polynucleotide. In some embodiments, the double stranded DNA binding moiety does not comprise a polypeptide.

In aspects of compositions suitable for treatment of genetic diseases provided herein, the double stranded DNA binding moiety has a molecular weight of less than about 15 kDa, less than about 12.5 kDa, less than about 10 kDa, less than about 9.5 kDa, less than about 9 kDa, less than about 8.5 kDa, less than about 8 kDa, less than about 7.5 kDa, less than about 7 kDa, less than about 6.5 kDa, less than about 6 kDa, less than about 5.5 kDa, less than about 5 kDa, less than about 4.5 kDa, less than about 4 kDa, less than about 3.5 kDa, less than about 3 kDa, less than about 2.5 kDa, less than about 2 kDa, or less than about 1.5 kDa. In some embodiments, the double stranded DNA binding moiety has a molecular weight of less than about 10 kDa, about 7.5 kDa, about 5 kDa, or about 2.5 kDa.

In another aspect of compositions suitable for treating genetic diseases provided herein, the composition comprises a double stranded DNA binding moiety, wherein the double stranded DNA binding moiety is a polymer that does not comprise nucleotides or amino acids.

In some embodiments of compositions suitable for treating genetic diseases, the double stranded DNA binding moiety comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker.

### DNA-Binding Moiety

In aspects of compositions suitable for treatment of genetic diseases provided herein, the double stranded DNA binding moiety has a structure of Formula (A-1), or a pharmaceutically acceptable salt thereof.

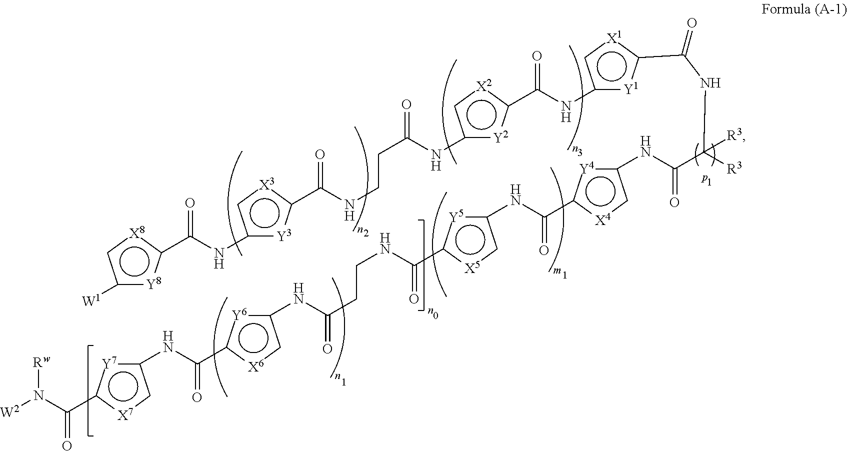

wherein:
each X¹, X², X³, X⁴, X⁵, X⁶, X⁷, and X⁸ is independently O or NR²;
each Y¹, Y², Y³, Y⁴, Y⁵, Y⁶, Y⁷, and Y⁸ is independently CH or N;
W¹ is hydrogen, halogen, optionally substituted C₁-C₁₀ alkyl, —N(R^{1e})₂, —NR^{1e}C(O)R^{1f}, —C(O)NR^{1e}R^{1f}, —N=C(N(R^{1e})₂)₂, —NR^{1e}C(O)R^{1f}, —OC(O)NR^{1e}R^{1f}, AA₁₋₁₀, —Z_{B}—P(O)(OR^{1e})₂, —Z_{B}—(CH₂)^{p3}—P(O)(OR^{1e})₂, or —Z_{B}—(CH₂)ₚ₃—O—P(O)(OR^{1e})₂, wherein
   each R^{1e} is independently hydrogen, optionally substituted C₁-C₂₀ alkyl, optionally substituted C₁-C₂₀ heteroalkyl, or PEG₁₋₂₀;
   each R^{1f} is independently hydrogen, optionally substituted C₁-C₂₀ alkyl, optionally substituted C₁-C₂₀ heteroalkyl, optionally substituted 5-membered heteroaryl, PEG₁₋₂₀, or AA₁₋₁₀;
   each AA is independently a naturally occurring amino acid;
   Z_{B} is N or O; and
   p₃ is 1-10;
W² is hydrogen, optionally substituted C₁-C₂₀ alkyl, or optionally substituted C₁-C₂₀ heteroalkyl; or W² is -L¹-Z—R⁴, wherein
   U is alkylene or heteroalkylene;
   Z is absent, —C(O)—, or —C(═NH)—; and
   R⁴ is C₁-C₆ alkyl, —OR^{4b}, or —NR^{4a}R^{4b}; wherein
      R^{4a} is hydrogen, optionally substituted C₁-C₂₀ alkyl, or optionally substituted C₁-C₂₀ heteroalkyl;
      R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl; or
      R^{4a} and R^{4b} together with the nitrogen to which they are attached form an optionally substituted 4 to 8-membered heterocycloalkyl which is partially or fully unsaturated;
R^{W} is hydrogen or optionally substituted C₁-C₂₀ alkyl; or
W² and R^{W} together with the nitrogen to which they are attached form an optionally substituted 4 to 8-membered heterocycloalkyl which is partially or fully unsaturated;
each R² is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, optionally substituted C₁-C₂₀ hydroxyalkyl, optionally substituted C₁-C₂₀ aminoalkyl, optionally substituted C₁-C₂₀ haloalkyl, optionally substituted C₃-C₈ cycloalkyl, optionally substituted 3 to 8-membered heterocycloalkyl, or optionally substituted PEG₁₋₂₀;
each R³ is independently hydrogen, halogen, acetyl, amino, amido, hydroxy, optionally substituted C₁-C₂₀ alkyl, optionally substituted C₁-C₂₀ haloalkyl, optionally substituted C₁-C₂₀ alkylamino, or optionally substituted C₁-C₂₀ hydroxyalkyl; or
two R³ together with the atom(s) to which they are attached form a C₃-C₆ cycloalkyl or 4 to 6-membered heterocycloalkyl;
p₁ is 3 or 4;
n₁ and n₂ are each independently 0 or 1;
n₃ is 0, 1, or 2;
m₁ is 0, 1, 2, or 3; and
n₀ is 0 or 1, wherein no and m₁ are both not 0.

In some embodiments of Formula (A-1), n₀ is 1. In some embodiments of Formula (A-1), n₀ is 0.

In some embodiments of Formula (A-1), n₂ is 1. In some embodiments of Formula (A-1), n₂ is 0.

In some embodiments of Formula (A-1), n₃ is 2. In some embodiments of Formula (A-1), n₃ is 1. In some embodiments of Formula (A-1), n₃ is 0.

In some embodiments of Formula (A-1), p₁ is 3. In some embodiments of Formula (A-1), p₁ is 4.

In some embodiments of Formula (A-1), each X¹, X², X³, X⁴, X⁵, X⁶, X⁷, and X⁸ is independently NR².

In some embodiments of Formula (A-1), each R² is independently hydrogen or an optionally substituted C₁-C₂₀ alkyl, each of which is optionally substituted with one or more amino, amido, azido, cyano, ester, oxo (=O), urea, optionally substituted aryl, or optionally substituted 5 to 10-membered heteroaryl. In some embodiments of Formula (A-1), each R² is independently an optionally substituted C₁-C₁₀ alkyl. In some embodiments of Formula (A-1), each R² is independently methyl, ethyl, n-propyl, isopropyl, n-butyl, isobutyl, sec-butyl, tert-butyl, pentyl, or hexyl. In some embodiments of Formula (A-1), each R² is methyl. In some embodiments of Formula (A-1), each R² is hydrogen.

In some embodiments of Formula (A-1), each R³ is independently hydrogen, amino, or amido. In some embodiments of Formula (A-1), each R³ is independently amino. In some embodiments of Formula (A-1), each R³ is independently amido. In some embodiments of Formula (A-1), each R³ is hydrogen.

In some embodiments of Formula (A-1), two R³ together with the atom(s) to which they are attached form a C₃-C₆ cycloalkyl or 4 to 6-membered heterocycloalkyl. In some embodiments of Formula (A-1), two R³ together with the atom(s) to which they are attached form a C₃-C₆ cycloalkyl. In some embodiments of Formula (A-1), two R³ together with the atom(s) to which they are attached form a 4 to 6-membered heterocycloalkyl.

In some embodiments of Formula (A-1), W² is -L¹-Z—R⁴.

In some embodiments of Formula (A-1), R^{W} is hydrogen.

In various aspects of compositions suitable for treatment of genetic diseases provided herein, the double stranded DNA binding moiety has a structure of Formula (A-2), or a pharmaceutically acceptable salt thereof:

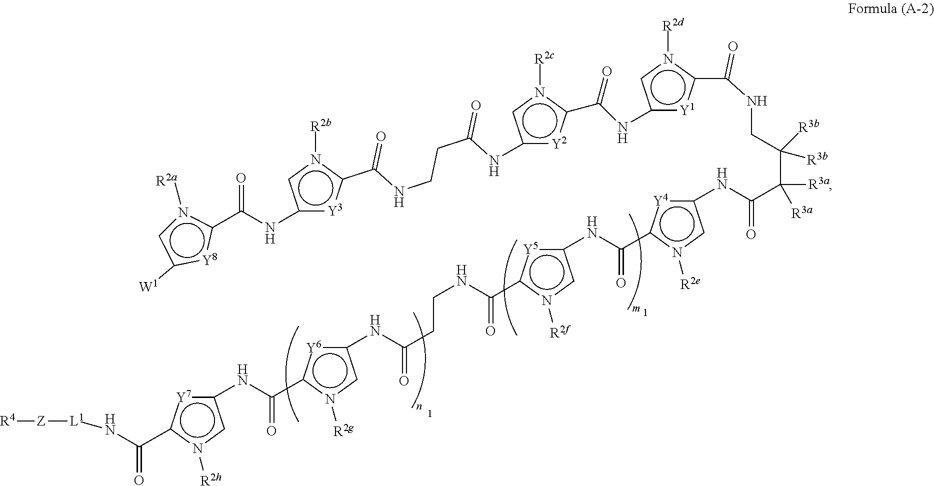

wherein:
W¹ is hydrogen or —N═C(N(R^{1e})₂)₂, wherein each R^{1e} is independently hydrogen or C₁-C₃ alkyl; each Y¹, Y², Y³, Y⁴, Y⁵, Y⁶, Y⁷, and Y⁸ is independently N or CH;
L¹ is C₁-C₂₀ alkylene or C₂-C₂₀ heteroalkylene;
Z is absent, —C(O)—, or —C(═NH)—;
R⁴ is C₁-C₆ alkyl, —OR^{4b}, or —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl which is partially or fully unsaturated;
each R^{2a}, R^{2b}, R^{2c}, R^{2d}, R^{2e}, R^{2f}, R^{2g}, and R^{2h} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, optionally substituted C₁-C₂₀ hydroxyalkyl, optionally substituted C₁-C₂₀ aminoalkyl, optionally substituted C₁-C₂₀ haloalkyl, optionally substituted C₃-C₈ cycloalkyl, optionally substituted 3 to 8-membered heterocycloalkyl, or optionally substituted PEG₁₋₂₀; each of which is optionally substituted with one or more R^{X};
each R^{3a} and R^{3b} is independently hydrogen, halogen, C₁-C₁₀ alkyl, —OR^{3e}, —NR^{3c}R^{3d}, or —NHC(O)R^{3e}, wherein
   R^{3c} and R^{3d} are each independently hydrogen, alkyl, or PEG;
   R^{3e} is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R^{X} is independently —CN, —OH, —OR^{Xa}, —N₃, —NR^{Xa}R^{Xb}, —CO(O)R^{Xc}, —C(O)OR^{Xc}, —C(O)NR^{Xa}R^{Xb}, —NHC(O)R^{Xc}, —NHC(O)OR^{Xc}, —OC(O)NR^{Xa}R^{Xb}, or optionally substituted 5 to 10-membered heteroaryl; wherein
   R^{Xa} and R^{Xb} are each independently hydrogen, alkyl, or PEG;
   R^{Xc} is alkyl, PEG, cycloalkyl, heterocycloalkyl, or phenyl; and
n₁ and m₁ are each independently 0 or 1.

In some embodiments of Formula (A-1) or (A-2), each Y², Y⁴, Y⁷, and Y⁸ is independently N. In some embodiments of Formula (A-1) or (A-2), each Y¹, Y³, and Y⁶ is independently CH.

In various aspects of compositions suitable for treatment of genetic diseases provided herein, the double stranded DNA binding moiety has a structure of Formula (A-3), or a pharmaceutically acceptable salt thereof:

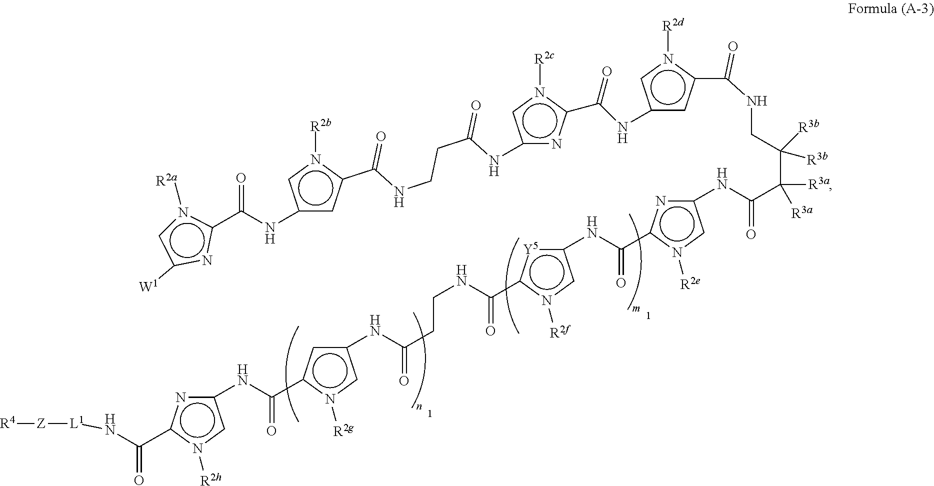

wherein:
W¹ is hydrogen or —N═C(N(R^{1e})₂)₂, wherein each R^{1e} is independently hydrogen or C₁-C₃ alkyl; each Y⁵ is CH or N;
L¹ is C₁-C₂₀ alkylene or C₂-C₂₀ heteroalkylene;
Z is absent, —C(O)—, or —C(═NH)—;
R⁴ is C₁-C₆ alkyl, —OR^{4b}, or —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₇ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl which is partially or fully unsaturated;
each R^{2a}, R^{2b}, R^{2c}, R^{2d}, R^{2e}, R^{2f}, R^{2g}, and R^{2h} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, optionally substituted C₁-C₂₀ hydroxyalkyl, optionally substituted C₁-C₂₀ aminoalkyl, optionally substituted C₁-C₂₀ haloalkyl, optionally substituted C₃-C₈ cycloalkyl, optionally substituted 3 to 8-membered heterocycloalkyl, or optionally substituted PEG₁₋₂₀; each of which is optionally substituted with one or more R^{X};
each R^{3a} and R^{3b} is independently hydrogen, halogen, C₁-C₁₀ alkyl, —OR^{3e}, —NR^{3c}R^{3d}, or —NHC(O)R^{3e}, wherein
   R^{3c} and R^{3d} are each independently hydrogen, alkyl, or PEG;
   R^{3e} is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R^{X} is independently —CN, —OH, —OR^{Xa}, —N₃, —NR^{Xa}R^{Xb}, —CO(O)R^{Xc}, —C(O)OR^{Xc}, —C(O)NR^{Xa}R^{Xb}, —NHC(O)R^{Xc}, —NHC(O)OR^{Xc}, —OC(O)NR^{Xa}R^{Xb}, or optionally substituted 5 to 10-membered heteroaryl; wherein
   R^{Xa} and R^{Xb} are each independently hydrogen, alkyl, or PEG;
   R^{Xc} is alkyl, PEG, cycloalkyl, heterocycloalkyl, or phenyl; and
n₁ and m₁ are each independently 0 or 1.

In various aspects of compositions suitable for treatment of genetic diseases provided herein, the double stranded DNA binding moiety has a structure of Formula (A-4), or a pharmaceutically acceptable salt thereof:

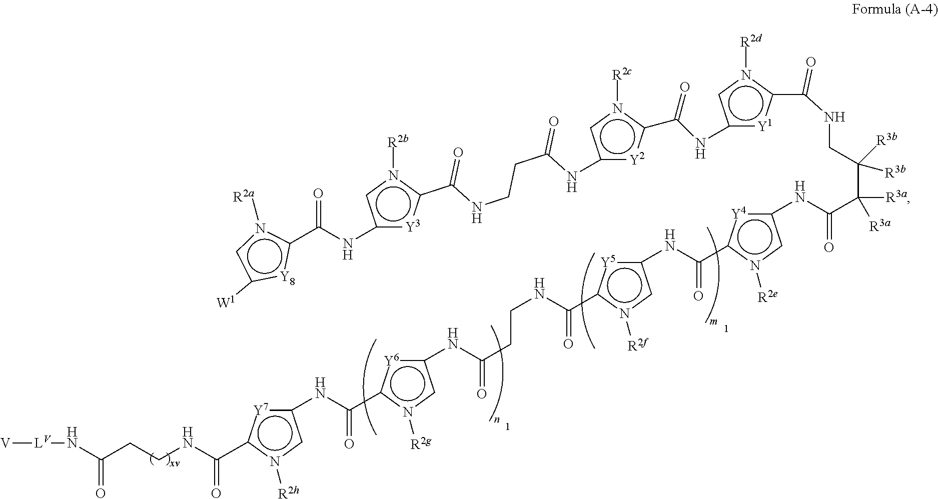

wherein:
W¹ is hydrogen or —N═C(N(R^{1e})₂)₂, wherein each R^{1e} is independently hydrogen or C₁-C₃ alkyl;
each Y¹, Y², Y³, Y⁴, Y⁵, Y⁶, Y⁷, and Y⁸ is independently N or CH;
L^{V} is C₁-C₂₀ alkylene, C₂-C₂₀ heteroalkylene, or AA₁₋₁₀; wherein each AA is independently a naturally occurring amino acid;
V is absent, optionally substituted C₃-C₈ cycloalkyl, optionally substituted 4 to 8-membered heterocycloalkyl, optionally substituted phenyl, or optionally substituted 5 to 10-membered heteroaryl;
each R^{2a}, R^{2b}, R^{2c}, R^{2d}, R^{2e}, R^{2f}, R^{2g}, and R^{2h} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, optionally substituted C₁-C₂₀ hydroxyalkyl, optionally substituted C₁-C₂₀ aminoalkyl, optionally substituted C₁-C₂₀ haloalkyl, optionally substituted C₃-C₈ cycloalkyl, optionally substituted 3 to 8-membered heterocycloalkyl, or optionally substituted PEG₁₋₂₀; each of which is optionally substituted with one or more R^{X};
each R^{3a} and R^{3b} is independently hydrogen, halogen, C₁-C₁₀ alkyl, —OR^{3e}, —NR^{3c}R^{3d}, or —NHC(O)R^{3e}, wherein
   R^{3c} and R^{3d} are each independently hydrogen, alkyl, or PEG;
   R^{3e} is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R^{X} is independently —CN, —OH, —OR^{Xa}, —N₃, —NR^{Xa}R^{Xb}, —CO(O)R^{Xc}, —C(O)OR^{Xc}, —C(O)NR^{Xa}R^{Xb}, —NHC(O)R^{Xc}, —NHC(O)OR^{Xc}, —OC(O)NR^{Xa}R^{Xb}, or optionally substituted 5 to 10-membered heteroaryl; wherein
   R^{Xa} and R^{Xb} are each independently hydrogen, alkyl, or PEG;
   R^{Xc} is alkyl, PEG, cycloalkyl, heterocycloalkyl, or phenyl;
n₁ and m₁ are each independently 0 or 1; and
xv is 0-10.

In some embodiments of Formula (A-4), each Y², Y⁴, Y⁷, and Y⁸ is independently N. In some embodiments of Formula (A-4), each Y¹, Y³, and Y⁶ is independently CH.

In some embodiments of Formula (A-4), L^{V} is C₁-C₂₀ alkylene or C₂-C₂₀ heteroalkylene. In some embodiments of Formula (A-4), L^{V} is C₁-C₂₀ alkylene. In some embodiments of Formula (A-4), L^{V} is C₁-C₁₀ alkylene. In some embodiments of Formula (A-4), L^{V} is C₂-C₂₀ heteroalkylene. In some embodiments of Formula (A-4), L^{V} is C₂-C₁₀ heteroalkylene. In some embodiments of Formula (A-4), L^{V} is PEG₁₋₁₀.

In some embodiments of Formula (A-4), xv is 0-8, 0-6, 0-4, or 0-2. In some embodiments of Formula (A-4), xv is 0-8. In some embodiments of Formula (A-4), xv is 0-4. In some embodiments of Formula (A-4), xv is 0, 1, 2, or 3.

In some embodiments, V is an optionally substituted C₃-C₈ cycloalkyl or optionally substituted 4 to 8-membered heterocycloalkyl. In some embodiments, V is an optionally substituted C₃-C₈ cycloalkyl. In some embodiments, V is an optionally substituted 4 to 8-membered heterocycloalkyl. In some embodiments, V is an optionally substituted 6-membered heterocycloalkyl. In some embodiments, V is an optionally substituted phenyl or optionally substituted 5 to 10-membered heteroaryl. In some embodiments, V is an optionally substituted phenyl. In some embodiments, V is an optionally substituted 5 to 10-membered heteroaryl. In some embodiments, V is absent.

In some embodiments of Formula (A-4), V has the structure of Formula (C-1), or a pharmaceutically acceptable salt thereof:

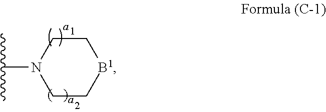

wherein:
B¹ is —CR⁶⁰R⁶⁰—, —O—, —NR⁶⁰—, —S(O)—, —S(O)₂—, or —S—; or B¹ is

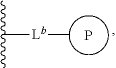

wherein
   each R⁶⁰ is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR^{60a}, or —C(O)R^{60a}, wherein R^{60a} is s hydrogen, optionally substituted C₁-C₂₀ alkyl, optionally substituted C₁-C₁₀ haloalkyl, optionally substituted PEG₁₋₂₀, optionally substituted C₃-C₆ cycloalkyl, optionally substituted 3 to 6-membered heterocycloalkyl, or optionally substituted phenyl; or
   two R⁶⁰ together with the nitrogen atom to which they are attached form an optionally substituted 4 to 8-membered heterocycloalkyl;
   Ring P is an optionally substituted C₃-C₆ cycloalkyl, optionally substituted 4 to 6-membered heterocycloalkyl, optionally substituted phenyl, or optionally substituted 5 to 10-membered heteroaryl;
   L^{b} is absent, C₁-C₄ alkylene, C₂-C₄ alkenylene, or C₂-C₄ alkynylene; and
   a₁ and a₂ are each independently 0, 1, or 2.

In some embodiments of Formula (C-1), B¹ is —CR⁶⁰R⁶⁰—, —O—, —NR⁶⁰—, —S(O)—, —S(O)₂—, or —S—. In some embodiments of Formula (C-1), B¹ is

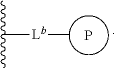

In some embodiments of Formula (C-1), each R⁶⁰ is independently hydrogen, optionally substituted C₁-C₂₀ alkyl, optionally substituted C₂-C₂₀ alkenyl, optionally substituted C₂-C₂₀ alkynyl, or optionally substituted C₁-C₂₀ heteroalkyl. In some embodiments of Formula (C-1), each R⁶⁰ is independently —C(O)OR^{60a} or —C(O)R^{60a}. In some embodiments of Formula (C-1), each R⁶⁰ is hydrogen.

In some embodiments of Formula (C-1), two R⁶⁰ together with the nitrogen atom to which they are attached form an optionally substituted 4 to 8-membered heterocycloalkyl.

In some embodiments of Formula (A-4), V has the structure of Formula (C-2), or a pharmaceutically acceptable salt thereof:

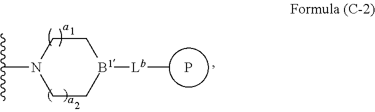

wherein:
Ring P is an optionally substituted C₃-C₆ cycloalkyl, optionally substituted 4 to 6-membered heterocycloalkyl, optionally substituted phenyl, or optionally substituted 5 to 10-membered heteroaryl;
L^{b} is absent, C₁-C₄ alkylene, C₂-C₄ alkenylene, or C₂-C₄ alkynylene;
B¹′ is CH or N; and
a₁ and a₂ are each independently 0, 1, or 2.

In some embodiments of Formula (C-2), Ring P is an optionally substituted C₃-C₆ cycloalkyl or optionally substituted 4 to 6-membered heterocycloalkyl. In some embodiments of Formula (C-2), Ring P is an optionally substituted C₃-C₆ cycloalkyl. In some embodiments of Formula (C-2), Ring P is an optionally substituted 4 to 6-membered heterocycloalkyl. In some embodiments of Formula (C-2), Ring P is an optionally substituted 6-membered heterocycloalkyl.

In some embodiments of Formula (C-1) or (C-2), a₁ and a₂ are each independently 0 or 1. In some embodiments of Formula (C-1) or (C-2), a₁ and a₂ are each 1. In some embodiments of Formula (C-1) or (C-2), a₁ and a₂ are each independently 0. In some embodiments of Formula (C-1) or (C-2), a₁ is 0 and a₂ is 1.

In some embodiments of Formula (A-4), V has the structure of Formula (C-3), or a pharmaceutically acceptable salt thereof:

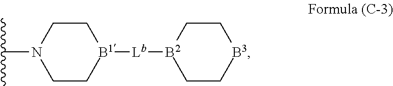

wherein:
B¹′ and B² are each independently CH or N; and
B³ is —CR⁶¹R⁶¹—, —O—, —S—, —S(O)—, —S(O)₂—, or —NR⁶¹—; wherein
   each R⁶¹ is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR^{61a}, or —C(O)R^{61a}; wherein R^{61a} is hydrogen, optionally substituted C₁-C₂₀ alkyl, optionally substituted C₁-C₁₀ haloalkyl, optionally substituted PEG₁₋₂₀, optionally substituted C₃-C₆ cycloalkyl, optionally substituted 3 to 6-membered heterocycloalkyl, or optionally substituted phenyl; and
L^{b} is absent, C₁-C₄ alkylene, C₂-C₄ alkenylene, or C₂-C₄ alkynylene.

In some embodiments of Formula (C-2) or (C-3), B¹′ is CH. In some embodiments, of Formula (C-2) or (C-3), B¹′ is N.

In some embodiments of Formula (C-1), (C-2), or (C-3), L^{b} is C₁-C₄ alkylene, C₂-C₄ alkenylene, or C₂-C₄ alkynylene. In some embodiments of Formula (C-1), (C-2), or (C-3), L^{b} is absent or C₂-C₄ alkynylene. In some embodiments of Formula (C-1), (C-2), or (C-3), L^{b} is C₂-C₄ alkynylene. In some embodiments of Formula (C-1), (C-2), or (C-3), L^{b} is absent.

In some embodiments of Formula (A-4), V has the structure of Formula (C-4), or a pharmaceutically acceptable salt thereof

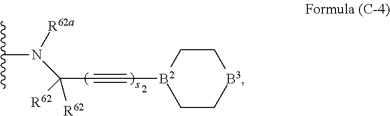

wherein:
B² is CH or N;
B³ is —CR⁶¹R⁶¹—, —O—, —S—, —S(O)—, —S(O)₂—, or —NR⁶¹—; wherein
   each R⁶¹ is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR^{61a}, or —C(O)R^{61a}; wherein R^{61a} is hydrogen, optionally substituted C₁-C₂₀ alkyl, optionally substituted C₁-C₁₀ haloalkyl, optionally substituted PEG₁₋₂₀, optionally substituted C₃-C₆ cycloalkyl, optionally substituted 3 to 6-membered heterocycloalkyl, or optionally substituted phenyl; and
R^{62a} is hydrogen, optionally substituted C₁-C₂₀ alkylene, or optionally substituted PEG₁₋₂₀;
each R⁶² is independently hydrogen or C₁-C₃ alkyl; and
s₂ is 1, 2, or 3.

In some embodiments of Formula (C-3) or (C-4), B² is CH. In some embodiments of Formula (C-3) or (C-4), B² is N.

In some embodiments of Formula (C-3) or (C-4), B³ is —CR⁶¹R⁶¹—, —O—, or —NR⁶¹—. In some embodiments of Formula (C-3) or (C-4), B³ is —CR⁶¹R⁶¹—. In some embodiments of Formula (C-3) or (C-4), B³ is —O—. In some embodiments of Formula (C-3) or (C-4), B³ is —NR⁶¹—. In some embodiments of Formula (C-3) or (C-4), B³ is —S—, —S(O)—, or —S(O)₂—.

In some embodiments of Formula (C-3) or (C-4), each R⁶¹ is independently hydrogen, optionally substituted C₁-C₂₀ alkyl, optionally substituted C₂-C₂₀ alkenyl, optionally substituted C₂-C₂₀ alkynyl, or optionally substituted C₁-C₂₀ heteroalkyl. In some embodiments of Formula (C-3) or (C-4), each R⁶¹ is independently —C(O)OR^{61a} or —C(O)R^{61a}. In some embodiments of Formula (C-3) or (C-4), each R⁶¹ is hydrogen.

In some embodiments of Formula (C-3) or (C-4), R^{61a} is an optionally substituted C₁-C₂₀ alkyl. In some embodiments of Formula (C-3) or (C-4), R^{61a} is an optionally substituted phenyl. In some embodiments of Formula (C-3) or (C-4), R^{61a} is optionally substituted PEG₁₋₂₀. In some embodiments of Formula (C-3) or (C-4), R^{61a} is hydrogen.

In some embodiments of Formula (C-4), each R⁶² is independently C₁-C₃ alkyl. In some embodiments of Formula (C-4), each R⁶² is hydrogen.

In some embodiments of Formula (C-4), R^{62a} is optionally substituted C₁-C₂₀ alkylene or optionally substituted PEG₁₋₂₀. In some embodiments of Formula (C-4), R^{62a} is hydrogen.

In some embodiments of Formula (C-4), s₂ is 1 or 2. In some embodiments of Formula (C-4), s₂ is 2. In some embodiments of Formula (C-4), s₂ is 1.

In some embodiments, of Formula (A-2), (A-3), or (A-4), each R^{2a}, R^{2b}, R^{2c}, R^{2d}, R^{2e}, R^{2f}, R^{2g}, and R^{2h} is independently hydrogen, optionally substituted C₁-C₂₀ alkyl, optionally substituted C₁-C₂₀ heteroalkyl, optionally substituted C₁-C₂₀ hydroxyalkyl, optionally substituted C₁-C₂₀ aminoalkyl, optionally substituted C₁-C₂₀ haloalkyl, optionally substituted C₃-C₈ cycloalkyl, optionally substituted 3 to 8-membered heterocycloalkyl, or optionally substituted PEG₁₋₂₀; each of which is optionally substituted with one or more R^{X}. In some embodiments of Formula (A-2), (A-3), or (A-4), each R^{2a}, R^{2b}, R^{2c}, R^{2d}, R^{2e}, R^{2f}, R^{2g}, and R^{2h} is independently hydrogen, optionally substituted C₁-C₂₀ alkyl, optionally substituted C₁-C₂₀ haloalkyl, optionally substituted C₁-C₂₀ heteroalkyl, optionally substituted C₃-C₈ cycloalkyl, optionally substituted 4 to 8-membered heterocycloalkyl, each of which is optionally substituted with one or more R^{X}. In some embodiments of Formula (A-2), (A-3), or (A-4), each R^{2a}, R^{2b}, R^{2c}, R^{2d}, R^{2e}, R^{2f}, R^{2g}, and R^{2h} is independently hydrogen, optionally substituted C₁-C₂₀ alkyl, optionally substituted C₁-C₂₀ haloalkyl, optionally substituted C₃-C₈ cycloalkyl, optionally substituted 4 to 8-membered heterocycloalkyl, each of which is optionally substituted with one or more R^{X}. In some embodiments of Formula (A-2), (A-3), or (A-4), each R^{2a}, R^{2b}, R^{2c}, R^{2d}, R^{2e}, R^{2f}, R^{2g}, and R^{2h} is independently hydrogen or optionally substituted C₁-C₂₀ alkyl, each of which is optionally substituted with one or more R^{X}. In some embodiments of Formula (A-2), (A-3), or (A-4), each R^{2a}, R^{2b}, R^{2c}, R^{2d}, R^{2e}, R^{2f}, R^{2g}, and R^{2h} is independently optionally substituted C₃-C₈ cycloalkyl or optionally substituted 4 to 8-membered heterocycloalkyl. In some embodiments of Formula (A-2), (A-3), or (A-4), each R^{2a}, R^{2b}, R^{2c}, R^{2d}, R^{2e}, R^{2f}, R^{2g}, and R^{2h} is independently optionally substituted C₃-C₈ cycloalkyl. In some embodiments of Formula (A-2), (A-3), or (A-4), each R^{2a}, R^{2b}, R^{2c}, R^{2d}, R^{2e}, R^{2f}, R^{2g}, and R^{2h} is independently optionally substituted 4 to 8-membered heterocycloalkyl. In some embodiments of Formula (A-2), (A-3), or (A-4), each R^{2a}, R^{2b}, R^{2c}, R^{2d}, R^{2e}, R^{2f}, R^{2g}, and R^{2h} is independently optionally substituted C₁-C₂₀ heteroalkyl. In some embodiments of Formula (A-2), (A-3), or (A-4), each R^{2a}, R^{2b}, R^{2c}, R^{2d}, R^{2e}, R^{2f}, R^{2g}, and R^{2h} is independently optionally substituted C₁-C₁₀ alkyl. In some embodiments of Formula (A-2), (A-3), or (A-4), each R^{2a}, R^{2b}, R^{2c}, R^{2d}, R^{2e}, R^{2f}, R^{2g}, and R^{2h} is independently methyl, ethyl, n-propyl, isopropyl, n-butyl, isobutyl, sec-butyl, tert-butyl, pentyl, or hexyl. In some embodiments of Formula (A-2), (A-3), or (A-4), each R^{2a}, R^{2b}, R^{2c}, R^{2d}, R^{2e}, R^{2f}, R^{2g}, and R^{2h} is methyl. In some embodiments of Formula (A-2), (A-3), or (A-4), each R^{2a}, R^{2b}, R^{2c}, R^{2d}, R^{2e}, R^{2f}, R^{2g}, and R^{2h} is hydrogen.

In some embodiments of Formula (A-2), (A-3), or (A-4), each R^{3a} and R^{3b} is independently hydrogen, —NR^{3c}R^{3d}, or —NHC(O)R^{3f}. In some embodiments of Formula (A-2), (A-3), or (A-4), each R^{3a} and R^{3b} is independently hydrogen or —NR^{3c}R^{3d}. In some embodiments of Formula (A-2), (A-3), or (A-4), each R^{3a} and R^{3b} is independently hydrogen or —NH₂. In some embodiments of Formula (A-2), (A-3), or (A-4), each R^{3a} and R^{3b} is hydrogen.

In some embodiments of Formula (A-2), (A-3), or (A-4), two R^{3a} together with the carbon atom to which they are attached form a C₃-C₆ cycloalkyl. In some embodiments of Formula (A-2), (A-3), or (A-4), two R^{3a} together with the carbon atom to which they are attached form a cyclopropyl, cyclobutyl, or cyclopentyl. In some embodiments of Formula (A-2), (A-3), or (A-4), two R^{3a} together with the carbon atom to which they are attached form a cyclopropyl. In some embodiments of Formula (A-2), (A-3), or (A-4), two R^{3a} together with the carbon atom to which they are attached form a cyclobutyl. In some embodiments of Formula (A-2), (A-3), or (A-4), two R^{3a} together with the carbon atom to which they are attached form a cyclopentyl. In some embodiments of Formula (A-2), (A-3), or (A-4), two R^{3a} together with the carbon atom to which they are attached form a 4 to 6-membered heterocycloalkyl.

In some embodiments of Formula (A-2), (A-3), or (A-4), two R^{3b} together with the carbon atom to which they are attached form a C₃-C₆ cycloalkyl. In some embodiments of Formula (A-2), (A-3), or (A-4), two R^{3b} together with the carbon atom to which they are attached form a cyclopropyl, cyclobutyl, or cyclopentyl. In some embodiments of Formula (A-2), (A-3), or (A-4), two R^{3b} together with the carbon atom to which they are attached form a cyclopropyl. In some embodiments of Formula (A-2), (A-3), or (A-4), two R^{3b} together with the carbon atom to which they are attached form a cyclobutyl. In some embodiments of Formula (A-2), (A-3), or (A-4), two R^{3b} together with the carbon atom to which they are attached form a cyclopentyl. In some embodiments of Formula (A-2), (A-3), or (A-4), two R^{3b} together with the carbon atom to which they are attached form a 4 to 6-membered heterocycloalkyl.

In some embodiments of Formula (A-1), (A-2), or (A-3), L¹ is C₁-C₁₀ alkylene or C₂-C₁₀ heteroalkylene. In some embodiments of Formula (A-1), (A-2), or (A-3), L¹ is C₁-C₁₀ alkylene. In some embodiments, L¹ is C₂-C₁₀ heteroalkylene. In some embodiments of Formula (A-1), (A-2), or (A-3), the heteroalkylene is polyethylene glycol. In some embodiments of Formula (A-1), (A-2), or (A-3), L¹ is PEG₁₋₁₀. In some embodiments of Formula (A-1), (A-2), or (A-3), L¹ is —(CH₂CH₂—O)_{y1}—, wherein y₁ is an integer in the range of 1-10. In some embodiments of Formula (A-1), (A-2), or (A-3), the heteroalkylene comprises —(CH₂)ₓ₃N(R^{a})(CH₂)ₓ₄—, wherein R^{a} is hydrogen or an optionally substituted C₁-C₆ alkyl; and each x₃ and x₄ is independently an integer in the range of 1-6.

In some embodiments of Formula (A-1), (A-2), or (A-3), Z is —C(O)—; and R⁴ is —NR^{4a}R^{4b}. In some embodiments of Formula (A-1), (A-2), or (A-3), Z is absent; and R⁴ is —OR^{4b}. In some embodiments of Formula (A-1), (A-2), or (A-3), Z is absent; and R⁴ is —NR^{4a}R^{4b}. In some embodiments of Formula (A-1), (A-2), or (A-3), Z is —C(O)—; and R⁴ is optionally substituted C₁-C₆ alkyl. In some embodiments of Formula (A-1), (A-2), or (A-3), Z is absent; and R⁴ is optionally substituted C₁-C₆ alkyl.

In some embodiments of Formula (A-1), (A-2), or (A-3), R^{4a} is hydrogen, optionally substituted C₁-C₂₀ alkyl, or optionally substituted C₁-C₂₀ heteroalkyl. In some embodiments of Formula (A-1), (A-2), or (A-3), R^{4a} is an optionally substituted C₁-C₂₀ alkyl or optionally substituted C₁-C₂₀ heteroalkyl. In some embodiments of Formula (A-1), (A-2), or (A-3), R^{4a} is an optionally substituted C₁-C₂₀ alkyl. In some embodiments of Formula (A-1), (A-2), or (A-3), R^{4a} is an optionally substituted C₁-C₂₀ heteroalkyl. In some embodiments of Formula (A-1), (A-2), or (A-3), the heteroalkyl is polyethylene glycol (PEG). In some embodiments of Formula (A-1), (A-2), or (A-3), R^{4a} is optionally substituted PEG₁₋₂₀. In some embodiments of Formula (A-1), (A-2), or (A-3), R^{4a} is hydrogen.

In some embodiments of Formula (A-1), (A-2), or (A-3), R^{4b} is hydrogen, optionally substituted C₁-C₂₀ alkyl, or optionally substituted C₁-C₂₀ heteroalkyl. In some embodiments of Formula (A-1), (A-2), or (A-3), R^{4b} is an optionally substituted C₁-C₂₀ alkyl or optionally substituted C₁-C₂₀ heteroalkyl. In some embodiments of Formula (A-1), (A-2), or (A-3), R^{4b} is an optionally substituted C₁-C₂₀ alkyl. In some embodiments of Formula (A-1), (A-2), or (A-3), R^{4b} is an optionally substituted C₁-C₂₀ heteroalkyl. In some embodiments of Formula (A-1), (A-2), or (A-3), the heteroalkyl is polyethylene glycol (PEG). In some embodiments of Formula (A-1), (A-2), or (A-3), R^{4b} is optionally substituted PEG₁₋₂₀. In some embodiments of Formula (A-1), (A-2), or (A-3), R^{4b} is hydrogen.

In some embodiments of Formula (A-1), (A-2), or (A-3), R^{4b} is optionally substituted C₃-C₈ cycloalkyl, optionally substituted 4 to 8-membered heterocycloalkyl, optionally substituted phenyl, or optionally substituted 5 to 10-membered heteroaryl. In some embodiments of Formula (A-1), (A-2), or (A-3), R^{4b} is optionally substituted C₃-C₈ cycloalkyl or optionally substituted 4 to 8-membered heterocycloalkyl. In some embodiments of Formula (A-1), (A-2), or (A-3), R^{4b} is optionally substituted C₃-C₆ cycloalkyl. In some embodiments of Formula (A-1), (A-2), or (A-3), R^{4b} is optionally substituted 4 to 6-membered heterocycloalkyl.

In some embodiments of Formula (A-1), (A-2), or (A-3), R^{4a} and R^{4b} together with the nitrogen to which they are attached form an optionally substituted 4 to 8-membered heterocycloalkyl which is partially or fully unsaturated. In some embodiments of Formula (A-1), (A-2), or (A-3), R^{4a} and R^{4b} together with the nitrogen to which they are attached form an optionally substituted 4 to 6-membered heterocycloalkyl. In some embodiments of Formula (A-1), (A-2), or (A-3), R^{4a} and R^{4b} together with the nitrogen to which they are attached form an optionally substituted piperidine, piperazine, or morpholine.

In some embodiments of Formula (A-2), (A-3), or (A-4), each R^{X} is independently —CN, —OH, —OR^{Xa}, —N₃, —NR^{Xa}R^{Xb}, —C(O)OR^{Xc}, —C(O)NR^{Xa}R^{Xb}, or —NHC(O)R^{Xc}. In some embodiments of Formula (A-2), (A-3), or (A-4), each R^{X} is independently —CN, —OH, —OR^{Xa}, —N₃, or —NR^{Xa}R^{Xb}. In some embodiments of Formula (A-2), (A-3), or (A-4), each R^{X} is independently —CO(O)R^{Xc}, —C(O)OR^{Xc}, —C(O)NR^{Xa}R^{Xb}, or —NHC(O)R^{Xc}.

In some embodiments of Formula (A-2), (A-3), or (A-4), each R^{Xa} and R^{Xb} is independently hydrogen, C₁-C₂₀ alkyl, or PEG₁₋₂₀. In some embodiments of Formula (A-2), (A-3), or (A-4), each R^{Xa} and R^{Xb} is independently C₁-C₂₀ alkyl. In some embodiments of Formula (A-2), (A-3), or (A-4), each R^{Xa} and R^{Xb} is independently PEG₁₋₂₀. In some embodiments of Formula (A-2), (A-3), or (A-4), each R^{Xa} and R^{Xb} is hydrogen.

In some embodiments of Formula (A-2), (A-3), or (A-4), R^{Xc} is C₁-C₂₀ alkyl, PEG₁₋₂₀, C₃-C₆ cycloalkyl, 4 to 6-membered heterocycloalkyl, or phenyl. In some embodiments of Formula (A-2), (A-3), or (A-4), R^{Xc} is C₁-C₂₀ alkyl or PEG₁₋₂₀. In some embodiments of Formula (A-2), (A-3), or (A-4), R^{Xc} is C₁-C₂₀ alkyl. In some embodiments of Formula (A-2), (A-3), or (A-4), R^{Xc} is PEG₁₋₂₀.

In some embodiments of Formula (A-2), (A-3), or (A-4), R^{3c} and R^{3d} are each independently hydrogen, C₁-C₂₀ alkyl, or PEG₁₋₂₀. In some embodiments of Formula (A-2), (A-3), or (A-4), R^{3c} and R^{3d} are each independently hydrogen or C₁-C₂₀ alkyl. In some embodiments of Formula (A-2), (A-3), or (A-4), R^{3c} and R^{3d} are each independently hydrogen.

In some embodiments of Formula (A-2), (A-3), or (A-4), R^{3e} is C₁-C₂₀ alkyl, PEG₁₋₂₀, C₃-C₆ cycloalkyl, 4 to 6-membered heterocycloalkyl, or phenyl. In some embodiments of Formula (A-2), (A-3), or (A-4), R^{3e} is C₁-C₂₀ alkyl or PEG₁₋₂₀.

In some embodiments of Formula (A-1), (A-2), (A-3), or (A-4), each Y⁵ is independently N. In some embodiments of Formula (A-1), (A-2), (A-3), or (A-4), each Y⁵ is independently CH.

In some embodiments of Formula (A-1), (A-2), (A-3), or (A-4), W¹ is —N═C(N(R^{1e})₂)₂, wherein each R^{1e} is independently hydrogen or C₁-C₃ alkyl. In some embodiments of Formula (A-1), (A-2), (A-3), or (A-4), W¹ is hydrogen.

In some embodiments of Formula (A-1), (A-2), (A-3), or (A-4), n₁ is 1. In some embodiments of Formula (A-1), (A-2), (A-3), or (A-4), n₁ is 0.

In some embodiments of Formula (A-1), (A-2), (A-3), or (A-4), m₁ is 0 or 1. In some embodiments of Formula (A-1), (A-2), (A-3), or (A-4), m₁ is 1. In some embodiments of Formula (A-1), (A-2), (A-3), or (A-4), m₁ is 0.

In aspects of compositions suitable for treatment of genetic diseases provided herein, the double stranded DNA binding moiety is optionally linked to a second terminus comprising a moiety that modulates gene expression. In some embodiments, the double stranded DNA binding moiety is linked by way of a linker to a second terminus comprising a moiety that modulates gene expression.

In some embodiments, the second terminus comprising the moiety that modulates gene expression is conjugated off of a pyrrole on the double stranded DNA binding moiety. In some embodiments, the second terminus comprising the moiety that modulates gene expression is conjugated off of an imidazole on the double stranded DNA binding moiety. In some embodiments, the second terminus comprising the moiety that modulates gene expression is conjugated off of a beta alanine of the double stranded DNA binding moiety. In some embodiments, the second terminus comprising the moiety that modulates gene expression is conjugated off of the C-terminus of the double stranded DNA binding moiety. In some embodiments, the second terminus comprising the moiety that modulates gene expression is conjugated off of the N-terminus of the double stranded DNA binding moiety. In some embodiments, the second terminus comprising the moiety that modulates gene expression is conjugated off of an amide of the double stranded DNA binding moiety.

In aspects of compositions suitable for treatment of genetic diseases provided herein, the double stranded DNA binding moiety of Formula (A-1), (A-2), or (A-3) is optionally linked to a second terminus comprising a moiety that modulates gene expression by way of an oligomeric linker. In aspects of compositions suitable for treatment of genetic diseases provided herein, the double stranded DNA binding moiety of Formula (A-1) is optionally linked to a second terminus comprising a moiety that modulates gene expression. In aspects of compositions suitable for treatment of genetic diseases provided herein, the double stranded DNA binding moiety of Formula (A-2) is optionally linked to a second terminus comprising a moiety that modulates gene expression. In aspects of compositions suitable for treatment of genetic diseases provided herein, the double stranded DNA binding moiety of Formula (A-3) is optionally linked to a second terminus comprising a moiety that modulates gene expression.

In some embodiments, the second terminus comprising the moiety that modulates gene expression is conjugated at W¹. In some embodiments, the second terminus comprising the moiety that modulates gene expression is conjugated at W². In some embodiments, the second terminus comprising the moiety that modulates gene expression is conjugated at one of R². In some embodiments, the second terminus comprising the moiety that modulates gene expression is conjugated at one of R², R^{2b}, R^{2c}, R^{2d}, R^{2e}, R^{2f}, R^{2g}, or R^{2h}. In some embodiments, the second terminus comprising the moiety that modulates gene expression is conjugated at one of R³. In some embodiments, the second terminus comprising the moiety that modulates gene expression is conjugated at one of R^{3a}. In some embodiments, the second terminus comprising the moiety that modulates gene expression is conjugated at one of R^{3b}. In some embodiments, the second terminus comprising the moiety that modulates gene expression is conjugated at R⁴. In some embodiments, the second terminus comprising the moiety that modulates gene expression is conjugated at R^{4a} or R^{4b}.

Moiety that Modulates Gene Expression

In some embodiments, the second terminus comprises a moiety that modulates gene expression.

In some embodiments, the second terminus comprises a bromodomain binding moiety.

In some embodiments, the second terminus comprises a moiety capable of binding to a bromodomain and extra terminal domain (BET) family member.

In some embodiments, the BET family member is BRD2, BRD3, BRD4, or BRDT. In some embodiments, the BET family member is BRD2. In some embodiments, the BET family member is BRD3. In some embodiments, the BET family member is BRD4. In some embodiments, the BET family member is BRDT.

In some embodiments, the protein-binding moiety binds to CBP/p300, PCAF (P300/CBP-Associated Factor), CECR2 (cat eye syndrome chromosome region candidate 2), BRPF (bromodomain and PHD finger-containing protein), ATAD2/ATAD2B (chromatin remodeling proteins), TRIM24 (Tripartite motif-containing 24), BAZ2 (Bromodomain Adjacent to Zinc finger), TAF1 (TBP associated factors), BRD7/9, BPTF (Bromodomain PHD Finger Transcription Factor), SMARCA2/4, or PBRM1.

In some embodiments, the regulatory molecule is CBP/p300.

In some embodiments, the regulatory molecule is PCAF (P300/CBP-Associated Factor).

In some embodiments, the regulatory molecule is CECR2 (cat eye syndrome chromosome region candidate 2).

In some embodiments, the regulatory molecule is BRPF (bromodomain and PHD finger-containing protein).

In some embodiments, the regulatory molecule is an ATAD2 or ATAD2B chromatin remodeling protein.

In some embodiments, the regulatory molecule is BAZ2 (Bromodomain Adjacent Zinc Finger.

In some embodiments, the regulatory molecule is TAF1 (TBP associated factor).

In some embodiments, the regulatory molecule is TRIM24 (tripartite motif-containing 24).

In some embodiments, the regulatory molecule is BRD7/9.

In some embodiments, the regulatory molecule is BPTF (Bromodomain PHD Finger Transcription Factor).

In some embodiments, the regulatory molecule is SMARCA2/4.

In some embodiments, the regulatory molecule is PBRM1.

In some embodiments, the regulatory molecule modulates the rearrangement of histones.

In some embodiments, the regulatory molecule modulates the glycosylation, phosphorylation, alkylation, or acylation of histones.

In some embodiments, the regulatory molecule is a transcription factor.

In some embodiments, the regulatory molecule is an RNA polymerase.

In some embodiments, the regulatory molecule is a moiety that regulates the activity of RNA polymerase.

In some embodiments, the recruiting moiety binds to the regulatory molecule but does not inhibit the activity of the regulatory molecule. In some embodiments, the recruiting moiety binds to the regulatory molecule and inhibits the activity of the regulatory molecule. In some embodiments, the recruiting moiety binds to the regulatory molecule and increases the activity of the regulatory molecule.

In some embodiments, the recruiting moiety binds to the active site of the regulatory molecule. In certain embodiments, the recruiting moiety binds to a regulatory site of the regulatory molecule.

In some embodiments, the second terminus comprises the structure of Formula (2-A), or a pharmaceutically acceptable salt thereof:

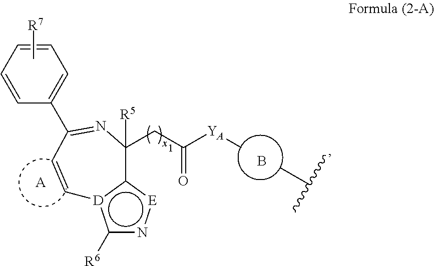

wherein:
Ring A is an optionally substituted aryl or optionally substituted 5 to 6-membered heteroaryl;
Ring B is absent or an optionally substituted 6-membered monocyclic aryl or heteroaryl;
D is C or N;
E is O or N;
Y_{A} is —NH— or —O—;
R⁵ is hydrogen or C₁-C₆ alkyl;
R⁶ is selected from hydrogen, optionally substituted C₁-C₆ alkyl, optionally substituted C₁-C₆ haloalkyl, and optionally substituted C₁-C₆ hydroxyalkyl;
R⁷ is selected from hydrogen, halogen, —NO₂, —CN, optionally substituted aryl, optionally substituted C₁-C₂₀ alkyl, optionally substituted C₁-C₂₀ heteroalkyl, optionally substituted C₁-C₆ haloalkyl, and optionally substituted C₁-C₆ hydroxyalkyl;
or R⁷ is —NR^{7A}R^{7B}, wherein R^{7A} and R^{7B} are each independently hydrogen, optionally substituted C₁-C₂₀ alkyl, or optionally substituted C₁-C₂₀ heteroalkyl; and
x₁ is an integer from 1-6.

In some embodiments, D is N and E is N. In some embodiments, D is C and E is O.

In some embodiments, the second terminus comprises the structure of Formula (2-B), or a pharmaceutically acceptable salt thereof:

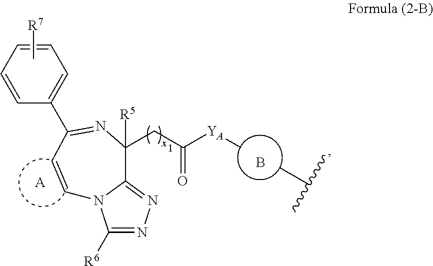

wherein:
Ring A is an optionally substituted aryl or optionally substituted 5 to 6-membered heteroaryl;
Ring B is absent or an optionally substituted 6-membered monocyclic aryl or heteroaryl;
Y_{A} is —NH— or —O—;
R⁵ is hydrogen or C₁-C₆ alkyl;
R⁶ is selected from hydrogen, optionally substituted C₁-C₆ alkyl, optionally substituted C₁-C₆ haloalkyl, and optionally substituted C₁-C₆ hydroxyalkyl;
R⁷ is selected from hydrogen, halogen, —NO₂, —CN, optionally substituted aryl, optionally substituted C₁-C₂₀ alkyl, optionally substituted C₁-C₂₀ heteroalkyl, optionally substituted C₁-C₆ haloalkyl, and optionally substituted C₁-C₆ hydroxyalkyl;
or R⁷ is —NR^{7A}R^{7B}, wherein R^{7A} and R^{7B} are each independently hydrogen, optionally substituted C₁-C₂₀ alkyl, or optionally substituted C₁-C₂₀ heteroalkyl; and
x₁ is an integer from 1-6.

In some embodiments, Ring A is an optionally substituted aryl ring. In some embodiments, Ring A is an optionally substituted phenyl. In some embodiments, Ring A is an optionally substituted 5 membered heteroaryl. In some embodiments, Ring A is an optionally substituted oxazolyl, optionally substituted furanyl, or optionally substituted thiophenyl.

In some embodiments, the second terminus comprising the structure of Formula (2-C), or a pharmaceutically acceptable salt thereof:

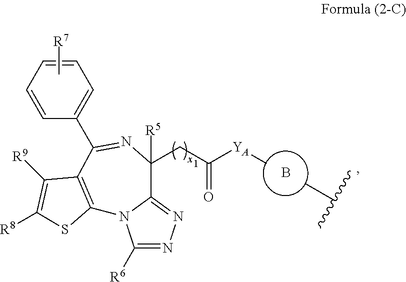

wherein:
R⁸ and R⁹ are each independently selected from hydrogen, optionally substituted C₁-C₆ alkyl, optionally substituted C₁-C₆ haloalkyl, or optionally substituted C₁-C₆ hydroxyalkyl.

In some embodiments, R⁸ and R⁹ are each independently selected from optionally substituted C₁-C₆ alkyl, C₁-C₆ haloalkyl, or C₁-C₆ hydroxyalkyl. In some embodiments, R⁸ and R⁹ are each independently selected from optionally substituted C₁-C₆ alkyl. In some embodiments, R⁸ and R⁹ are each independently methyl, ethyl, or propyl. In some embodiments, R⁸ and R⁹ are each independently methyl. In some embodiments, R⁸ and R⁹ are each independently ethyl. In some embodiments, R⁸ and R⁹ are each independently propyl.

In some embodiments, the second terminus comprising the structure of Formula (2-D), or a pharmaceutically acceptable salt thereof:

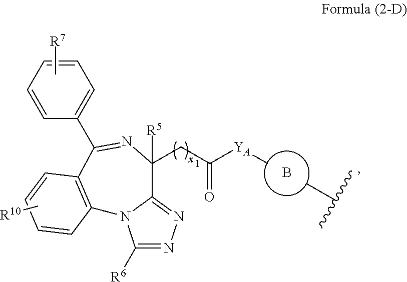

wherein:
R¹⁰ is selected from hydrogen, optionally substituted C₁-C₆ alkyl, optionally substituted C₁-C₆ haloalkyl, or optionally substituted C₁-C₆ hydroxyalkyl.

In some embodiments, R⁵ is C₁-C₆ alkyl. In some embodiments, R⁵ is methyl or ethyl. In some embodiments, R⁵ is methyl. In some embodiments, R is ethyl. In some embodiments, R⁵ is hydrogen.

In some embodiments, R⁷ is selected from hydrogen, halogen, optionally substituted C₁-C₆ alkyl, C₁-C₆ haloalkyl, or C₁-C₆ hydroxyalkyl. In some embodiments, R⁷ is halogen. In some embodiments, R⁷ is Br, Cl, or F. In some embodiments, R⁷ is Cl. In some embodiments, R⁷ is F. In some embodiments, R⁷ is Br.

In some embodiments, R⁷ is —NR^{7A}R^{7B}, wherein R^{7A} and R^{7B} are each independently hydrogen or optionally substituted C₁-C₆ alkyl.

In some embodiments, R¹⁰ is selected from optionally substituted C₁-C₆ alkyl, optionally substituted C₁-C₆ haloalkyl, or optionally substituted C₁-C₆ hydroxyalkyl. In some embodiments, R¹⁰ is selected from optionally substituted C₁-C₆ alkyl. In some embodiments, R¹⁰ is methyl, ethyl, or propyl. In some embodiments, R¹⁰ is methyl. In some embodiments, R¹⁰ is optionally substituted C₁₋₆ hydroxyalkyl. In some embodiments, R¹⁰ is —OMe.

In some embodiments, R⁶ is selected from optionally substituted C₁-C₆ alkyl, optionally substituted C₁-C₆ haloalkyl, or optionally substituted C₁-C₆ hydroxyalkyl. In some embodiments, R⁶ is an optionally substituted C₁-C₆ alkyl. In some embodiments, R⁶ is methyl, ethyl, or propyl. In some embodiments, R⁶ is methyl. In some embodiments, R⁶ is ethyl. In some embodiments, R⁶ is propyl. In some embodiments, R⁶ is hydrogen.

In some embodiments, Y_{A} is —NH—. In some embodiments, Y_{A} is —O—. In some embodiments, Y_{A} is NH and x₁ is 1.

In some embodiments, x₁ is an integer from 1-5, 1-4, 1-3, or 1-2. In some embodiments, x₁ is 1. In some embodiments, x₁ is 2.

In some embodiments, Ring B is an optionally substituted 6-membered monocyclic aryl or heteroaryl, each of which is optionally substituted with alkyl, amino, halogen, hydroxy, hydroxyalkyl, or PEG. In some embodiments Ring B is phenyl. In some embodiments, Ring B is 6-membered monocyclic heteroaryl. In some embodiments, Ring B is pyridine or pyrimidine. In some embodiments, ring B is absent.

In some embodiments, the second terminus comprises the structure of Formula (2-E), (2-F), or (2-G), or a pharmaceutically acceptable salt thereof:

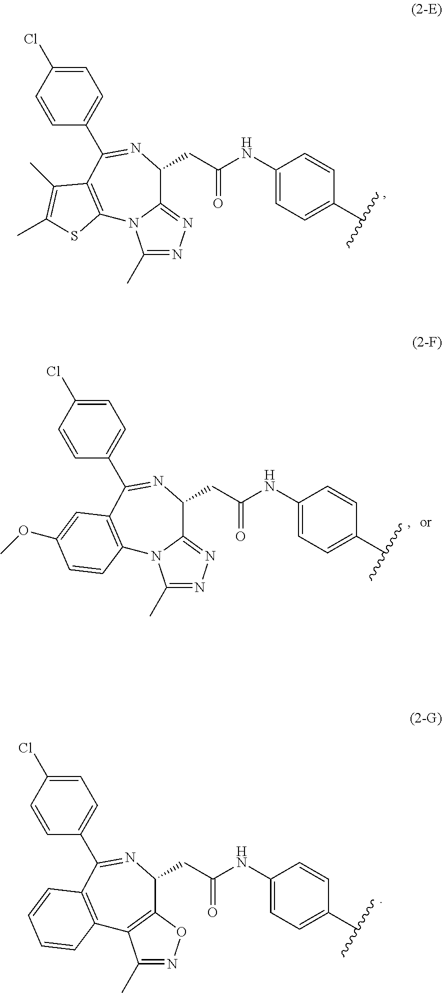 [SMILES: *c1ccc(NC(=O)C[C@H]2N=C(c3ccc(Cl)cc3)c3c(sc(C)c3C)-n3c(C)nnc32)cc1 ; *c1ccc(NC(=O)C[C@H]2N=C(c3ccc(Cl)cc3)c3cc(OC)ccc3-n3c(C)nnc32)cc1 ; *c1ccc(NC(=O)C[C@H]2N=C(c3ccc(Cl)cc3)c3ccccc3-c3c(C)noc32)cc1]

In some embodiments, the second terminus comprises the structure of Formula (3-A), or a pharmaceutically acceptable salt thereof:

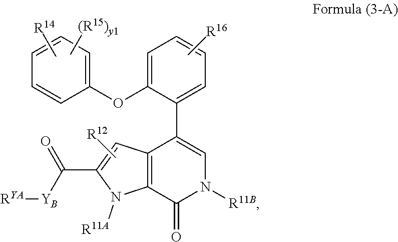

wherein:
Y_{B} is —CH₂NH—, —CH₂O—, —NH—, or —O—;
R^{11A} and R^{11B} are each independently hydrogen or optionally substituted C₁-C₆ alkyl;
R¹² is hydrogen, halogen, —OH, optionally substituted C₁-C₆ alkyl, optionally substituted C₁-C₆ haloalkyl, or optionally substituted C₁-C₆ hydroxyalkyl;
R¹⁴ and R¹⁵ are each independently hydrogen, halogen, —CN, —NO₂, optionally substituted C₁-C₆ alkyl, optionally substituted C₁-C₆ haloalkyl, or optionally substituted C₁-C₆ hydroxyalkyl; or R¹⁴ is —NR^{A}R^{B};
R¹⁶ is optionally substituted C₁-C₆ alkyl, optionally substituted C₁-C₆ heteroalkyl, optionally substituted C₂-C₆ alkenyl, optionally substituted C₂-C₆ alkynyl, C₁-C₆ hydroxyalkyl, —SO₂R^{A}, or —NHSO₂R^{A};
R^{YA} is hydrogen, optionally substituted C₁-C₆ alkyl, optionally substituted C₁-C₆ haloalkyl, or optionally substituted 5-6-membered monocyclic aryl or heteroaryl;
each R^{A} and R^{B} is independently hydrogen, optionally substituted C₁-C₆ alkyl, optionally substituted C₁-C₆ heteroalkyl, optionally substituted C₁-C₆ haloalkyl, optionally substituted C₁-C₆ hydroxyalkyl, optionally substituted C₃-C₆ cycloalkyl, optionally substituted 4 to 6-membered heterocycloalkyl, or optionally substituted 4 to 6-membered heteroalkyl; and
y₁ is 1-3;
wherein the attachment to the linker is at either R¹⁴ or at R^{YA}.

In some embodiments, the second terminus comprises the structure of Formula (3-B), or a pharmaceutically acceptable salt thereof:

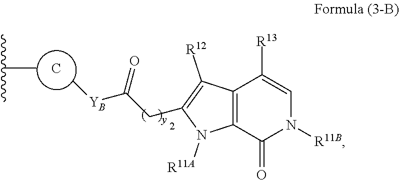

wherein:
Ring C is absent, optionally substituted 5 to 6-membered monocyclic aryl or heteroaryl or 4 to 8-membered heterocycle;
Y_{B} is —NH—, —CH₂NH—, —CH₂O—, or —O—;
R^{11A} and R^{11B} are each independently hydrogen or optionally substituted C₁-C₆ alkyl;
R¹² is hydrogen, optionally substituted C₁-C₆ alkyl, C(O)R^{A}, or C(O)NR^{A}R^{B}; wherein each R^{A} and R^{B} is independently hydrogen, optionally substituted C₁-C₆ alkyl, or optionally substituted C₁-C₆ heteroalkyl;
R¹³ is hydrogen, substituted aryl, substituted heteroaryl, or substituted oxydibenzene; and
y₂ is an integer from 0-2.

In some embodiments, y₂ is 0. In some embodiments, y₂ is 1. In some embodiments, y₂ is 2.

In some embodiments, R¹³ is substituted aryl or substituted heteroaryl. In some embodiments, R¹³ is hydrogen.

In some embodiments, R¹³ is substituted oxydibenzene.

In some embodiments, R¹³ is

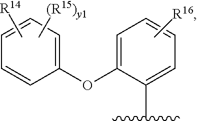

wherein
R¹⁴ and R¹⁵ are each independently hydrogen, halogen, —CN, —NO₂, optionally substituted C₁-C₆ alkyl, optionally substituted C₁-C₆ haloalkyl, or optionally substituted C₁-C₆ hydroxyalkyl;
or R¹⁴ is —NR^{A}R^{B};
R¹⁶ is an substituted C₁-C₆ alkyl, optionally substituted C₁-C₆ heteroalkyl, optionally substituted C₂-C₆ alkenyl, optionally substituted C₂-C₆ alkynyl, optionally substituted C₁-C₆ hydroxyalkyl, —SO₂R^{A}, or —NHSO₂R^{A};
each R^{A} and R^{B} is independently hydrogen, optionally substituted C₁-C₆ alkyl, optionally substituted C₁-C₆ heteroalkyl, optionally substituted C₁-C₆ haloalkyl, optionally substituted C₁-C₆ hydroxyalkyl, optionally substituted C₃-C₆ cycloalkyl, optionally substituted 4 to 6-membered heterocycloalkyl, or optionally substituted 5 to 6-membered heteroaryl; and
y₁ is 1-3.

In some embodiments, the second terminus comprises the structure of Formula (3-C), or a pharmaceutically acceptable salt thereof:

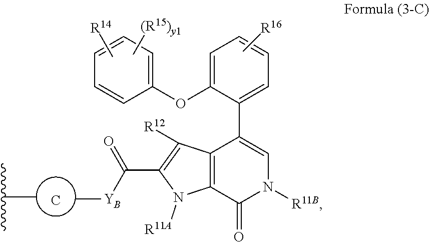

wherein:
Ring C is absent, optionally substituted 5 to 6-membered monocyclic aryl or heteroaryl or 4 to 8-membered heterocycle;
Y_{B} is —CH₂NH—, —CH₂O—, —NH—, or —O—;
R^{11A} and R^{11B} are each independently hydrogen or optionally substituted C₁-C₆ alkyl;
R¹² is hydrogen, halogen, —OH, optionally substituted C₁-C₆ alkyl, optionally substituted C₁-C₆ haloalkyl, or optionally substituted C₁-C₆ hydroxyalkyl;
R¹⁴ and R¹⁵ are each independently hydrogen, halogen, —CN, —NO₂, optionally substituted C₁-C₆ alkyl, optionally substituted C₁-C₆ haloalkyl, or optionally substituted C₁-C₆ hydroxyalkyl;
or R¹⁴ is —NR^{A}R^{B};
R¹⁶ is an substituted C₁-C₆ alkyl, optionally substituted C₁-C₆ heteroalkyl, optionally substituted C₂-C₆ alkenyl, optionally substituted C₂-C₆ alkynyl, optionally substituted C₁-C₆ hydroxyalkyl, —SO₂R^{A}, or —NHSO₂R^{A};
each R^{A} and R^{B} is independently hydrogen, optionally substituted C₁-C₆ alkyl, optionally substituted C₁-C₆ heteroalkyl, optionally substituted C₁-C₆ haloalkyl, optionally substituted C₁-C₆ hydroxyalkyl, optionally substituted C₃-C₆ cycloalkyl, optionally substituted 4 to 6-membered heterocycloalkyl, or optionally substituted 5 to 6-membered heteroaryl; and
y₁ is an integer from 1-3.

In some embodiments, Y_{B} is —NH—. In some embodiments, Y_{B} is —CH₂NH—. In some embodiments, Y_{B} is —CH₂O—. In some embodiments, Y_{B} is —O—.

In some embodiments, Ring C is an optionally substituted 5 or 6-membered monocyclic aryl or heteroaryl, each of which is optionally substituted with alkyl, amino, halogen, hydroxy, hydroxyalkyl, or PEG. In some embodiments, Ring C is phenyl. In some embodiments, Ring C is a 6-membered heteroaryl. In some embodiments, Ring C is pyridine, pyrazine, or triazine. In some embodiments, Ring C is pyridine. In some embodiments, Ring C is pyrazine. In some embodiments, Ring C is triazine. In some embodiments, Ring C is a 5-membered heteroaryl. In some embodiments, Ring C is a pyrazole. In some embodiments, Ring C is a triazole, pyrrole, imidazole, oxazole, oxadiazole, thiazole, or thiadiazole. In some embodiments, Ring C is a triazole. In some embodiments, Ring C is an imidazole or pyrrole. In some embodiments, an oxazole or oxadiazole. In some embodiments, Ring C is a thiazole or thiadiazole. In some embodiments, Ring C is absent.

In some embodiments, the second terminus comprises the structure of Formula (3-D), or a pharmaceutically acceptable salt thereof:

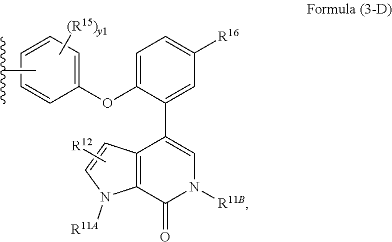

wherein:
R^{11A} and R^{11B} are each independently hydrogen or optionally substituted C₁-C₆ alkyl;
R¹² is hydrogen, halogen, —OH, optionally substituted C₁-C₆ alkyl, optionally substituted C₁-C₆ haloalkyl, optionally substituted C₁-C₆ hydroxyalkyl, C(O)R^{A}, or C(O)NR^{A}R^{B};
each R¹⁵ is independently hydrogen, halogen, —CN, —NO₂, optionally substituted C₁-C₆ alkyl, optionally substituted C₁-C₆ haloalkyl, or optionally substituted C₁-C₆ hydroxyalkyl;
R¹⁶ is an optionally substituted optionally substituted C₁-C₆ alkyl, optionally substituted C₁-C₆ heteroalkyl, optionally substituted C₂-C₆ alkenyl, optionally substituted C₂-C₆ alkynyl, optionally substituted C₁₋₆ hydroxyalkyl, —SO₂R^{A}, or —NHSO₂R^{A};
each R^{A} and R^{B} is hydrogen, optionally substituted C₁-C₆ alkyl, optionally substituted C₁-C₆ heteroalkyl, optionally substituted C₁-C₆ haloalkyl, optionally substituted C₁-C₆ hydroxyalkyl, optionally substituted C₃-C₆ cycloalkyl, optionally substituted 4 to 6-membered heterocycloalkyl, or optionally substituted 5 to 6-membered heteroaryl; and
y₁ is an integer from 1-3.

In some embodiments, R^{11A} and R^{11B} are each independently optionally substituted C₁-C₆ alkyl. In some embodiments, R^{11A} and R^{11B} are each independently methyl, ethyl, propyl, or tert-butyl. In some embodiments, R^{11A} and R^{11B} are each independently methyl. In some embodiments, R^{11A} and R^{11B} are each independently hydrogen.

In some embodiments, R^{11A} is C₁-C₆ alkyl, optionally substituted with haloalkyl or phosphorous hydroxide. In some embodiments, R^{11A} is C₁-C₆ alkyl substituted with —OP(O)(OH)₂. In some embodiments, R^{11A} is unsubstituted C₁-C₆ alkyl. In some embodiments, R^{11A} is methyl, ethyl, or tert-butyl. In some embodiments, R^{11A} is methyl. In some embodiments, R^{11A} is hydrogen.

In some embodiments, R¹² is optionally substituted C₁-C₆ alkyl. In some embodiments, R¹² is hydrogen. In some embodiments, R¹² is C(O)R^{A} or C(O)NR^{A}R^{B}. In some embodiments, R¹² is C(O)NR^{A}R^{B}, wherein R^{A} and R^{B} are each independently hydrogen or optionally substituted C₁-C₆ alkyl.

In some embodiments, R¹⁴ and R¹⁵ are each independently hydrogen, —CN, or —NO₂. In some embodiments, R¹⁴ and R¹⁵ are each independently halogen or optionally substituted C₁-C₆ alkyl. In some embodiments, R¹⁴ and R¹⁵ are each independently Br, Cl, F, methyl, or ethyl. In some embodiments, R¹⁴ and R¹⁵ are each independently F or methyl.

In some embodiments, R¹⁶ is optionally substituted optionally substituted C₁-C₆ alkyl, optionally substituted C₁-C₆ heteroalkyl, optionally substituted C₂-C₆ alkenyl, optionally substituted C₂-C₆ alkynyl, or optionally substituted C₁-C₆ hydroxyalkyl, each of which is optionally substituted with amido, alkyl, alkynyl, azido, amino, halogen, haloalkyl, hydroxy, nitro, oxo (=O), phosphorous hydroxide, or PEG. In some embodiments, R¹⁶ is optionally substituted optionally substituted C₁-C₆ alkyl, optionally substituted C₁-C₆ heteroalkyl, or optionally substituted C₁-C₆ hydroxyalkyl. In some embodiments, R¹⁶ is C₁-C₆ alkyl or C₁-C₆ heteroalkyl, each or which optionally substituted with —CN, —NH₂, —N₃, —OH, CF₃, or —OP(O)(OH)₂. In some embodiments, R¹⁶ is —SO₂R^{A}, wherein R^{A} is C₁-C₆ alkyl. In some embodiments, R¹⁶ is —SO₂Et. In some embodiments, R¹⁶ is —SO₂Me. In some embodiments, R¹⁶ is —NHSO₂R^{A}, wherein R^{A} is C₁-C₆ alkyl. In some embodiments, R¹⁶ is —NHSO₂Et. In some embodiments, R¹⁶ is —NHSO₂Me.

In some embodiments, y₁ is 1. In some embodiments, y₁ is 2. In some embodiments, y₁ is 3.

In some embodiments, the second terminus comprises the structure of Formula (3-E) or Formula (3-F), or a pharmaceutically acceptable salt thereof:

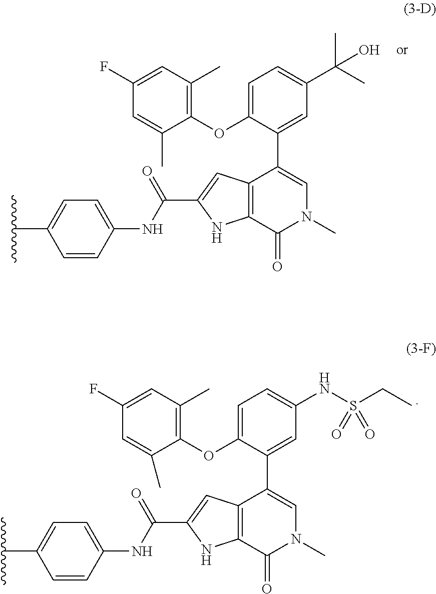 [SMILES: *c1ccc(NC(=O)c2cc3c(-c4cc(C(C)(C)O)ccc4Oc4c(C)cc(F)cc4C)cn(C)c(=O)c3[nH]2)cc1 ; *c1ccc(NC(=O)c2cc3c(-c4cc(NS(=O)(=O)CC)ccc4Oc4c(C)cc(F)cc4C)cn(C)c(=O)c3[nH]2)cc1]

In some embodiments, the second terminus comprising the structure of Formula (3-G) or Formula (3-H), or a pharmaceutically acceptable salt thereof:

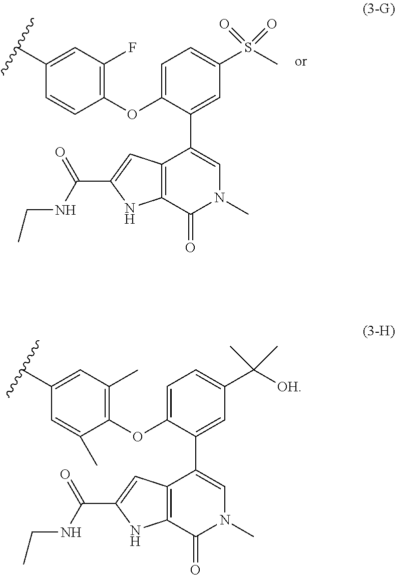 [SMILES: *c1ccc(Oc2ccc(S(C)(=O)=O)cc2-c2cn(C)c(=O)c3[nH]c(C(=O)NCC)cc23)c(F)c1 ; *c1cc(C)c(Oc2ccc(C(C)(C)O)cc2-c2cn(C)c(=O)c3[nH]c(C(=O)NCC)cc23)c(C)c1]

In some embodiments, the second terminus comprises the structure of Formula (4-A), or a pharmaceutically acceptable salt thereof:

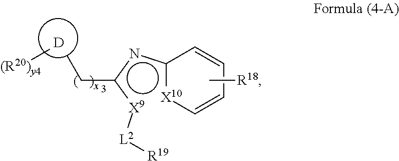

wherein:
Ring D is absent, phenyl, or 5 to 6-membered heteroaryl;
X⁹ and X¹⁰ are each independently C or N, wherein one of X⁹ or X¹⁰ is N;
L² is absent, optionally substituted alkylene, —O—, or —NR^{D}—, wherein R^{D} is hydrogen or optionally substituted C₁-C₃ alkyl;
R¹⁸ is an optionally substituted 5 to 6-membered heteroaryl;
R¹⁹ is an optionally substituted C₃-C₈ cycloalkyl or optionally substituted 4 to 7-membered heteroaryl;
each R²⁰ is independently hydrogen, halogen, —CN, —NO₂, optionally substituted C₁-C₆ alkyl, optionally substituted C₁-C₆ haloalkyl, or optionally substituted C₁-C₆ hydroxyalkyl;
x₃ is an integer from 1-3; and
y₄ is an integer from 1-4;
wherein the attachment to the linker is at either R¹⁹ or at one of R²⁰.

In some embodiments, attachment to the linker is at R¹⁹. In some embodiments, attachment to the linker is at one of R²⁰.

In some embodiments, the second terminus comprises the structure of Formula (4-B), or a pharmaceutically acceptable salt thereof:

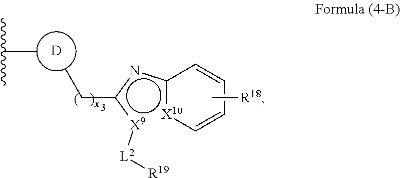

wherein:
Ring D is absent, optionally substituted phenyl, or optionally substituted 5 to 6-membered heteroaryl;
X⁹ and X¹⁰ are each independently C or N, wherein one of X⁹ or X¹⁰ is N;
L² is absent, optionally substituted alkylene, —O—, or —NR^{D}—, wherein R^{D} is hydrogen or optionally substituted C₁-C₃ alkyl;
R¹⁸ is an optionally substituted 5 to 6-membered heteroaryl;
R¹⁹ is an optionally substituted C₃-C₈ cycloalkyl or optionally substituted 4 to 7-membered heteroaryl; and
x₃ is an integer from 1-3.

In some embodiments, X⁹ is N; and X¹⁰ is C. In some embodiments, X⁹ is C; and X¹⁰ is N.

In some embodiments, the second terminus comprises the structure of Formula (4-C), or a pharmaceutically acceptable salt thereof:

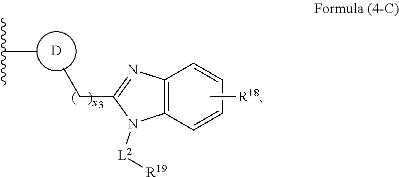

wherein:
Ring D is absent, optionally substituted phenyl, or optionally substituted 5 to 6-membered heteroaryl;
L² is absent, optionally substituted alkylene, —O—, or —NR^{D}—, wherein R^{D} is hydrogen or optionally substituted C₁-C₃ alkyl;
R¹⁸ is an optionally substituted 5 to 6-membered heteroaryl;
R¹⁹ is an optionally substituted C₃-C₈ cycloalkyl or optionally substituted 4 to 7-membered heteroaryl; and
x₃ is an integer from 1-3.

In some embodiments, Ring D is an optionally substituted monocyclic 6-membered aryl or 5 to 6-membered heteroaryl. In some embodiments, Ring D is an optionally substituted monocyclic 6-membered aryl. In some embodiments, Ring D is an optionally substituted phenyl.

In some embodiments, R¹⁹ is an optionally substituted C₃-C₈ cycloalkyl. In some embodiments, R¹⁹ is optionally substituted 4 to 7-membered heteroaryl.

In some embodiments, the second terminus comprises the structure of Formula (4-D), or a pharmaceutically acceptable salt thereof:

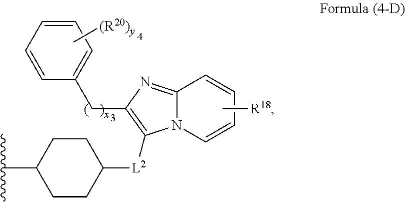

L² is an optionally substituted alkylene, —O—, or —NR^{D}—, wherein R^{D} is hydrogen or optionally substituted C₁-C₃ alkyl;
R¹⁸ is an optionally substituted 5 to 6-membered heteroaryl;
R²⁰ is hydrogen, halogen, —CN, —NO₂, optionally substituted C₁-C₆ alkyl, optionally substituted C₁-C₆ haloalkyl, or optionally substituted C₁-C₆ hydroxyalkyl;
x₃ is an integer from 1-3; and
y₄ is an integer from 1-4.

In some embodiments, L² is an optionally substituted alkylene. In some embodiments, L² is C₂-C₄ alkylene, optionally substituted with one or more C₁-C₃ alkyl. In some embodiments, L² is absent. In some embodiments, L² is —NR^{D}—. In some embodiments, L² is —NH—.

In some embodiments, R¹⁸ is an optionally substituted 5-membered heteroaryl. In some embodiments, R¹⁸ is optionally substituted oxazole, oxadiazole, thiazole, thiadiazole, pyrrole, or pyrazole. In some embodiments, R¹⁸ is optionally substituted oxazole.

In some embodiments, R²⁰ is halogen, —CN, —NO₂, optionally substituted C₁-C₆ alkyl, optionally substituted C₁-C₆ haloalkyl, or optionally substituted C₁-C₆ hydroxyalkyl.

In some embodiments, x₃ is 1. In some embodiments, x₃ is 2. In some embodiments, x₃ is 3.

In some embodiments, y₄ is 1 or 2. In some embodiments, y₄ is 1. In some embodiments, y₄ is 2. In some embodiments, y₄ is 3. In some embodiments, y₄ is 4.

In some embodiments, the second terminus comprises the structure of Formula (4-E), (4-F), or (4-G), or a pharmaceutically acceptable salt thereof:

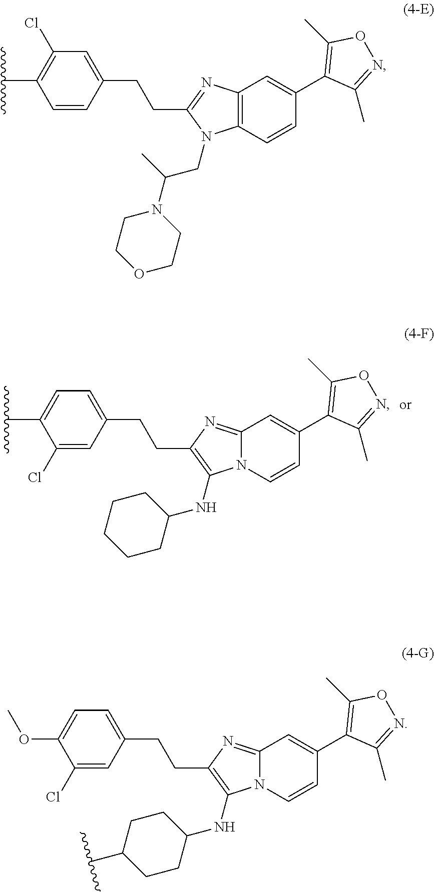 [SMILES: *c1ccc(CCc2nc3cc(-c4c(C)noc4C)ccc3n2CC(C)N2CCOCC2)cc1Cl ; *c1ccc(CCc2nc3cc(-c4c(C)noc4C)ccn3c2NC2CCCCC2)cc1Cl ; *C1CCC(Nc2c(CCc3ccc(OC)c(Cl)c3)nc3cc(-c4c(C)noc4C)ccn23)CC1]

In some embodiments, the second terminus comprises the structure of Formula (5-A), or a pharmaceutically acceptable salt thereof:

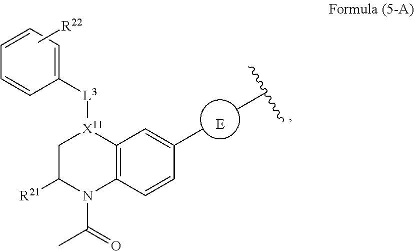

wherein:
Ring E is absent or an optionally substituted phenyl or optionally substituted 5 to 6-membered heteroaryl;
X¹¹ is CH or N;
L³ is —NR^{E}— or —CR^{E}R^{E}—, wherein each R^{E} is independently hydrogen or optionally substituted C₁-C₃ alkyl;
R²¹ is C₁-C₆ alkyl or C₃-C₆ cycloalkyl; and
R²² is halogen, CN, NO₂, optionally substituted C₁-C₆ alkyl, optionally substituted C₁-C₆ haloalkyl, or optionally substituted C₁-C₆ hydroxyalkyl.

In some embodiments, Ring E is absent. In some embodiments, Ring E is an optionally substituted phenyl. In some embodiments, Ring E is an optionally substituted 5 to 6-membered heteroaryl. In some embodiments, Ring E is a 5-membered heteroaryl. In some embodiments, Ring E is a 6-membered heteroaryl.

In some embodiments, X¹¹ is CH and L³ is —NR^{E}—. In some embodiments, X¹¹ is N and L³ is —CR^{E}R^{E}.

In some embodiments, R²¹ is C₁-C₆ alkyl. In some embodiments, R²¹ is methyl.

In some embodiments, R²² is halogen, optionally substituted C₁-C₆ alkyl, optionally substituted C₁-C₆ haloalkyl, or optionally substituted C₁-C₆ hydroxyalkyl. In some embodiments, R²² is CN, F, Cl, Br, or methyl.

In some embodiments, the second terminus comprises the structure of Formula (5-B), or a pharmaceutically acceptable salt thereof:

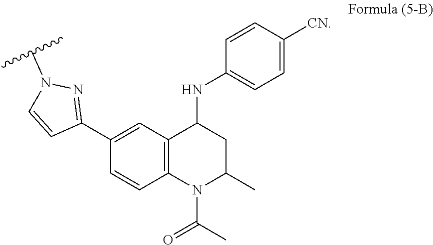 [SMILES: *n1ccc(-c2ccc3c(c2)C(Nc2ccc(C#N)cc2)CC(C)N3C(C)=O)n1]

In some embodiments, the second terminus comprises the structure of Formula (6-A), or a pharmaceutically acceptable salt thereof:

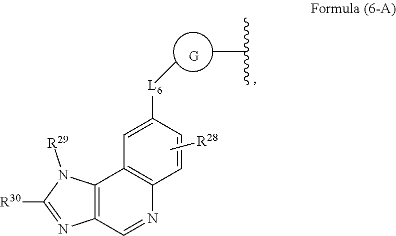

wherein:
Ring G is an optionally substituted C₃-C₆ cycloalkyl or optionally substituted 4 to 6-membered heterocycloalkyl;
L⁶ is —O-(optionally substituted alkylene);
R²⁸ is an optionally substituted 5 to 6-membered heteroaryl;
R²⁹ is optionally substituted C₁-C₆ alkyl(C₆-C₁₀ aryl) or optionally substituted C₁-C₆ alkyl(6 to 10-membered heteroaryl); and
R³⁰ is optionally substituted C₁-C₆ alkyl, optionally substituted C₁-C₆ haloalkyl, or optionally substituted C₁-C₆ hydroxyalkyl.

In some embodiments, the second terminus comprises the structure of Formula (7-A), or a pharmaceutically acceptable salt thereof:

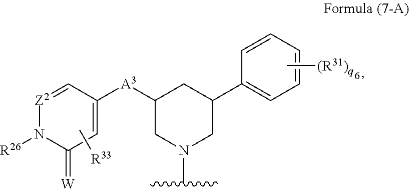

wherein:
A³ is —O—, —NH—, or —CH₂—;
Z² is CH or N;
W is O or S;
each R³¹ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈ cycloalkyl, or optionally substituted 3 to 8-membered heterocycloalkyl;
or two R³¹ together with the atoms to which they are attached join together to form an optionally substituted C₅-C₈ cycloalkyl or optionally substituted 5 to 8-membered heterocycloalkyl;
R³² is hydrogen or optionally substituted C₁-C₁₀ alkyl;
R³³ is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl; and
q₆ is 0-4.

In some embodiments, the second terminus comprises the structure of Formula (7-B), or a pharmaceutically acceptable salt thereof:

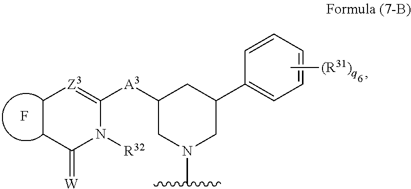

wherein:
Ring F is an optionally substituted 5 to 6-membered heteroaryl;
A³ is —O—, —NH—, or —CH₂—;
Z³ is CH or N;
W is O or S;
each R³¹ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3- to 8-membered heterocycloalkyl;
or two R³¹ together with the atoms to which they are attached form an optionally substituted C₅-C₈ cycloalkyl or optionally substituted 5 to 8-membered heterocycloalkyl;
R³² is hydrogen or optionally substituted C₁-C₁₀ alkyl; and
q₆ is 1-4.

In some embodiments, A³ is —O—. In some embodiments, A³ is —NH—. In some embodiments, A³ is —CH₂—.

In some embodiments, Z² is CH. In some embodiments, Z² is N.

In some embodiments, Z³ is CH. In some embodiments, Z³ is N.

In some embodiments, W is O. In some embodiments, W is S.

In some embodiments, Ring F is an optionally substituted 5-membered heteroaryl. In some embodiments, Ring F is an optionally substituted 6-membered heteroaryl.

In some embodiments, each R³¹ is independently an optionally substituted C₁-C₁₀ alkyl, optionally substituted C₁-C₁₀ haloalkyl, or optionally substituted C₁-C₁₀ hydroxyalkyl. In some embodiments, each R³¹ is independently an optionally substituted C₃-C₈-cycloalkyl or optionally substituted 3 to 8-membered heterocycloalkyl. In some embodiments, each R³¹ is independently hydrogen, halogen, —OH, —CN, —NO₂, or —NH₂. In some embodiments, each R³¹ is hydrogen.

In some embodiments, R³² is an optionally substituted C₁-C₁₀ alkyl. In some embodiments, R³² is methyl. In some embodiments, R³² is hydrogen.

In some embodiments, R³³ is hydrogen, halogen, —OH, —CN, —NO₂, or —NH₂. In some embodiments, R³³ is an optionally substituted C₁-C₁₀ alkyl, optionally substituted C₁-C₁₀ haloalkyl, or optionally substituted C₁-C₁₀ hydroxyalkyl.

In some embodiments, the second terminus comprises the structure of Formula (7-C), or a pharmaceutically acceptable salt thereof:

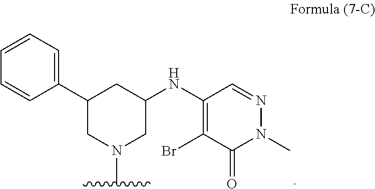 [SMILES: *N1CC(Nc2cnn(C)c(=O)c2Br)CC(c2ccccc2)C1]

In some embodiments, the second terminus comprise the structure of Formula (7-D), or a pharmaceutically acceptable salt thereof:

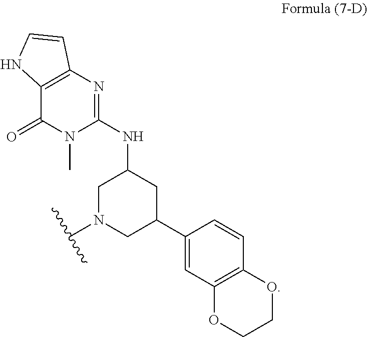 [SMILES: *N1CC(Nc2nc3cc[nH]c3c(=O)n2C)CC(c2ccc3c(c2)OCCO3)C1]

In some embodiments, the second terminus comprises the structure of Formula (8-A), or a pharmaceutically acceptable salt thereof:

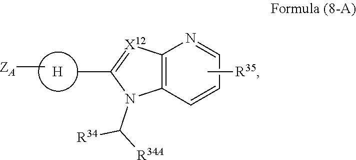

wherein:
Ring H is an optionally substituted phenyl or optionally substituted 6-membered heteroaryl;
or Ring H is

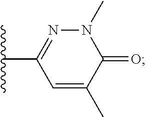 [SMILES: *c1cc(C)c(=O)n(C)n1]

Z_{A} is absent or an optionally substituted phenyl formamide;
X¹² is CH or N;
R³⁴ is an optionally substituted phenyl or optionally substituted 6-membered heteroaryl;
R^{34A} is hydrogen, halogen, or optionally substituted C₁-C₃ alkyl;
R³⁵ is independently halogen, optionally substituted C₁-C₁₀ alkyl, or optionally substituted 5 to 6-membered heteroaryl; and
wherein the attachment to the linker is at R³⁵, Z_{A}, or Ring H.

In some embodiments, Ring H is an optionally substituted phenyl. In some embodiments, Ring H is an optionally substituted 6-membered heteroaryl. In some embodiments, Ring H is

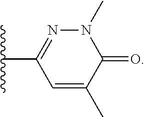 [SMILES: *c1cc(C)c(=O)n(C)n1]

In some embodiments, Z_{A} is absent. In some embodiments, Z_{A} is an optionally substituted phenyl formamide. In some embodiments, Z_{A} is —C(O)NH-phenyl.

In some embodiments, X¹² is CH. In some embodiments, X¹² is N.

In some embodiments, R³⁴ is an optionally substituted phenyl. In some embodiments, R³⁴ is an optionally substituted 6-membered heteroaryl.

In some embodiments, R^{34A} is hydrogen or halogen. In some embodiments, R^{34A} is an optionally substituted C₁-C₃ alkyl. In some embodiments, R^{34A} is methyl.

In some embodiments, Formula (8-A) is attachment to the linker is at R³⁵. In some embodiments, Formula (8-A) is attached to the linker at Z_{A}. In some embodiments, Formula (8-A) is attached to the linker at Ring H.

In some embodiments, the second terminus comprises the structure of Formula (8-B) or Formula (8-C), or a pharmaceutically acceptable salt thereof:

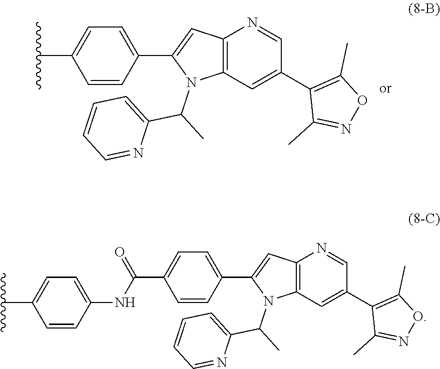 [SMILES: *c1ccc(-c2cc3ncc(-c4c(C)noc4C)cc3n2C(C)c2ccccn2)cc1 ; *c1ccc(NC(=O)c2ccc(-c3cc4ncc(-c5c(C)noc5C)cc4n3C(C)c3ccccn3)cc2)cc1]

In some embodiments, the second terminus comprises the structure of Formula (8-D), or a pharmaceutically acceptable salt thereof:

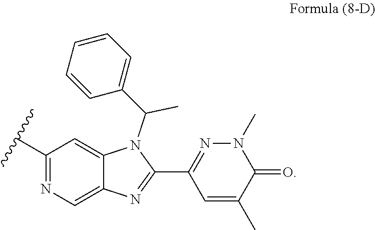 [SMILES: *c1cc2c(cn1)nc(-c1cc(C)c(=O)n(C)n1)n2C(C)c1ccccc1]

In some embodiments, the second terminus comprises the structure of Formula (9-A), or a pharmaceutically acceptable salt thereof:

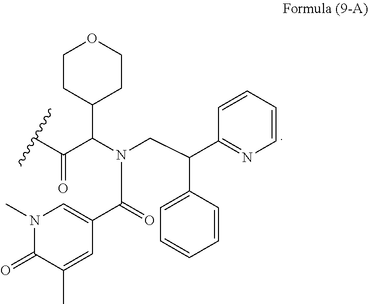 [SMILES: *C(=O)C(C1CCOCC1)N(CC(c1ccccc1)c1ccccn1)C(=O)c1cc(C)c(=O)n(C)c1]

In some embodiments, the second terminus comprises the structure of Formula (10-A) or Formula (10-B), or a pharmaceutically acceptable salt thereof:

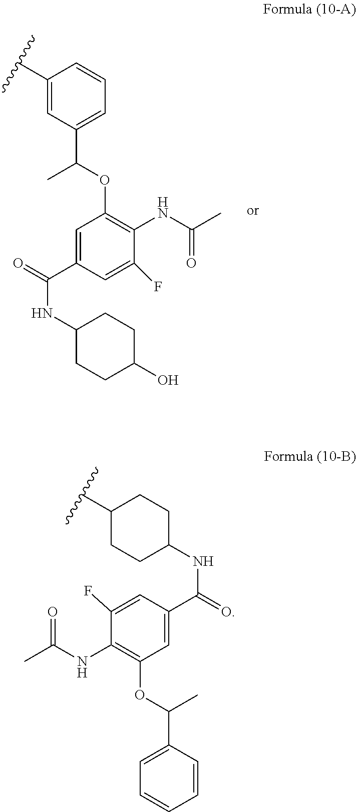

In some embodiments, the second terminus comprises the structure of Formula (11-A), or a pharmaceutically acceptable salt thereof:

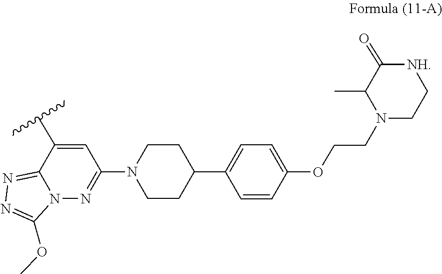 [SMILES: *c1cc(N2CCC(c3ccc(OCCN4CCNC(=O)C4C)cc3)CC2)nn2c(OC)nnc12]

In some embodiments, the second terminus comprises the structure of Formula (12-A), or a pharmaceutically acceptable salt thereof:

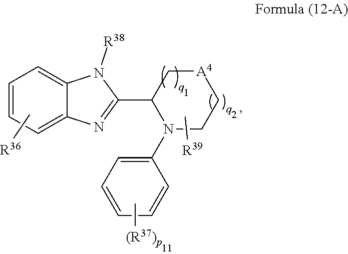

wherein:
A⁴ is —CR⁴⁰R⁴⁰— or —NR⁴⁰—, wherein each R⁴⁰ is independently hydrogen or optionally substituted C₁-C₁₀ alkyl;
R³⁶ is an optionally substituted 5 to 6-membered heteroaryl;
each R³⁷ is independently hydrogen, halogen, C₁-C₆ alkyl, or C₁-C₆ haloalkyl;
R³⁸ is optionally substituted C₁-C₁₀ alkyl, optionally substituted C₃-C₈ cycloalkyl, or optionally substituted 3 to 8-membered heterocycloalkyl;
R³⁹ is hydrogen, halogen, —OH, —CN, —NO₂, —NH₂, oxo (=O), ═S, C₁-C₁₀ haloalkyl, or C₁-C₁₀ hydroxyalkyl;
p₁₁ is 1-4;
q₁ and q₂ are each independently 0-2; and
wherein the linker is attached to Formula (12-A) at either R³⁸ or R⁴⁰.

In some embodiments, R³⁶ is an optionally substituted 5-membered heteroaryl. In some embodiments, R³⁶ is optionally substituted oxazole, oxadiazole, thiazole, thiadiazole, pyrrole, or pyrazole. In some embodiments, R³⁶ is optionally substituted oxazole.

In some embodiments, each R³⁷ is independently halogen, C₁-C₆ alkyl, or C₁-C₆ haloalkyl. In some embodiments, each R³⁷ is independently halogen.

In some embodiments, R³⁸ is an optionally substituted C₁-C₁₀ alkyl. In some embodiments, R³⁸ is an optionally substituted C₃-C₈ cycloalkyl or optionally substituted 3 to 8-membered heterocycloalkyl. In some embodiments, R³⁸ is a 3 to 8-membered heterocycloalkyl.

In some embodiments, R³⁹ is hydrogen, halogen, —OH, —CN, —NO₂, —NH₂, C₁-C₁₀ haloalkyl, or C₁-C₁₀ hydroxyalkyl. In some embodiments, R³⁹ is oxo or =S. In some embodiments, R³⁹ is oxo. In some embodiments, R³⁹ is =S.

In some embodiments, A⁴ is —NR⁴⁰. In some embodiments, A⁴ is —NH. In some embodiments, A⁶ is —NCH₃. In some embodiments, A⁴ is —CR⁴⁰R⁴⁰. In some embodiments, A⁴ is —CH₂—.

In some embodiments, each R⁴⁰ is independently optionally substituted C₁-C₁₀ alkyl. In some embodiments, each R⁴⁰ is independently hydrogen.

In some embodiments, p₁₁ is 3 or 4. In some embodiments, p₁₁ is 2. In some embodiments, p₁₁ is 1.

In some embodiments, q₁ is 1 and q₂ is 1. In some embodiments, q₁ is 2 and q₂ is 0.

In some embodiments, the linker is attached to Formula (12-A) through R³⁸. In some embodiments, the linker is attached to Formula (12-A) through R⁴⁰.

In some embodiments, the second terminus comprises Formula (12-B) or Formula (12-C), or a pharmaceutically acceptable salt thereof:

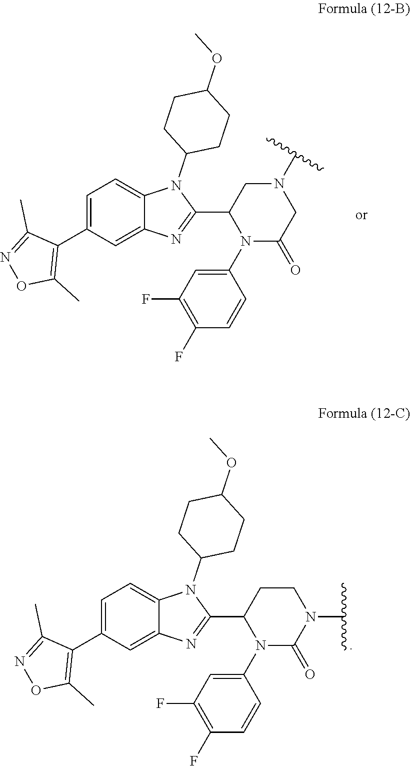 [SMILES: *N1CC(=O)N(c2ccc(F)c(F)c2)C(c2nc3cc(-c4c(C)noc4C)ccc3n2C2CCC(OC)CC2)C1 ; *N1CCC(c2nc3cc(-c4c(C)noc4C)ccc3n2C2CCC(OC)CC2)N(c2ccc(F)c(F)c2)C1=O]

In some embodiments, the second terminus comprises Formula (12-D) or Formula (12-E), or a pharmaceutically acceptable salt thereof:

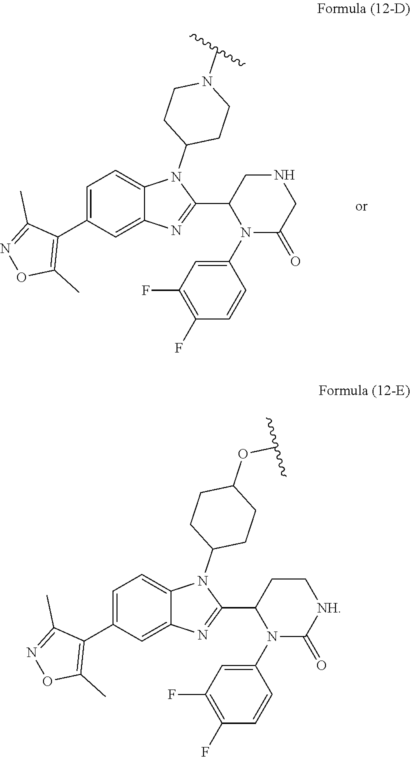 [SMILES: *N1CCC(n2c(C3CNCC(=O)N3c3ccc(F)c(F)c3)nc3cc(-c4c(C)noc4C)ccc32)CC1 ; *OC1CCC(n2c(C3CCNC(=O)N3c3ccc(F)c(F)c3)nc3cc(-c4c(C)noc4C)ccc32)CC1]

In some embodiments, the second terminus comprises the structure of Formula (13-A), or a pharmaceutically acceptable salt thereof:

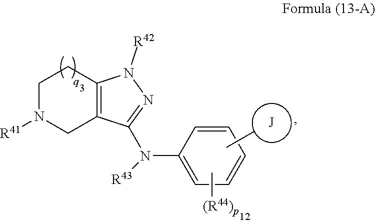

wherein:
Ring J is absent or optionally substituted 5 to 6-membered heteroaryl;
R⁴¹ is optionally substituted C₁-C₆ alkyl, optionally substituted C₃-C₈ cycloalkyl, —C(O)R^{41a}, —C(O)—, or —C(O)NR^{41a}R^{41b}, wherein
   R^{41a} and R^{41b} are each independently optionally substituted C₁-C₁₀ alkyl or optionally substituted C₃-C₈ cycloalkyl;
R⁴² is an optionally substituted C₁-C₁₀ alkyl, optionally substituted C₁-C₁₀ haloalkyl, optionally substituted C₃-C₈ cycloalkyl, or optionally substituted 3 to 8 membered heterocycloalkyl;
R⁴³ is hydrogen or optionally substituted C₁-C₁₀ alkyl;
each R⁴⁴ is independently hydrogen, halogen, —OH, —CN, —NO₂, —NH₂, optionally substituted C₁-C₁₀ alkyl, optionally substituted C₁-C₁₀ haloalkyl, C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3 to 8-membered heterocycloalkyl; or
R⁴³ and one of R⁴⁴ together with the atoms to which they are attached form an optionally substituted 5 to 8-membered heterocycloalkyl;
p₁₂ is 1-4;
q₃ is 0 or 1; and
wherein Formula (13-A) is connected to the linker at Ring J or at R⁴¹.

In some embodiments, R⁴¹ is optionally substituted C₁-C₆ alkyl or optionally substituted C₃-C₈ cycloalkyl. In some embodiments, R⁴¹ is —C(O)R^{41a}. In some embodiments, R⁴¹ is —C(O)CH₃ or —C(O)CH₂CH₃. In some embodiments, R⁴¹ is —C(O)—NR^{41a}R^{41b}.

In some embodiments, R^{41a} is optionally substituted C₁-C₁₀ alkyl. In some embodiments, R^{41a} is optionally substituted C₃-C₈ cycloalkyl.

In some embodiments, R^{41b} is optionally substituted C₁-C₁₀ alkyl. In some embodiments, R^{41b} is optionally substituted C₃-C₈ cycloalkyl.

In some embodiments, R⁴² is optionally substituted C₁-C₁₀ alkyl or optionally substituted C₁-C₁₀ haloalkyl. In some embodiments, R⁴² is optionally substituted C₃-C₈ cycloalkyl or optionally substituted 3 to 8 membered heterocycloalkyl. In some embodiments, R⁴² is optionally substituted 3- to 8-membered heterocycloalkyl ring.

In some embodiments, R⁴³ is optionally substituted C₁-C₁₀ alkyl. In some embodiments, R⁴³ is hydrogen.

In some embodiments, each R⁴⁴ is independently halogen, —OH, —CN, —NO₂, —NH₂, optionally substituted C₁-C₁₀ alkyl, optionally substituted C₁-C₁₀ haloalkyl, C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3 to 8-membered heterocycle. In some embodiments, each R⁴⁴ is independently halogen or C₁-C₁₀ haloalkyl.

In some embodiments, R⁴³ and one of R⁴⁴ together with the atoms to which they are attached form an optionally substituted 5 to 8-membered heterocycloalkyl. In some embodiments, R⁴³ and one of R⁴⁴ together with the atoms to which they are attached form a 5, 6, 7, or 8-membered heterocycloalkyl.

In some embodiments, p₁₂ is 3 or 4. In some embodiments, p₁₂ is 2. In some embodiments, p₁₂ is 1.

In some embodiments, q₃ is 1. In some embodiments, q₃ is 0.

In some embodiments, Ring J is an optionally substituted 5-membered heteroaryl. In some embodiments, Ring J is absent.

In some embodiments, Formula (13-A) is connected to the linker at Ring J. In some embodiments, Formula (13-A) is connected to the linker at R⁴¹.

In some embodiments, the second terminus comprises the structure of Formula (13-B), or a pharmaceutically acceptable salt thereof:

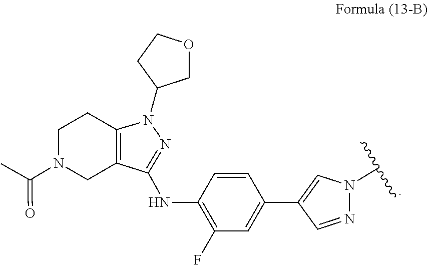

In some embodiments, the second terminus comprises the structure of Formula (13-C1) or Formula (13-C2), or a pharmaceutically acceptable salt thereof:

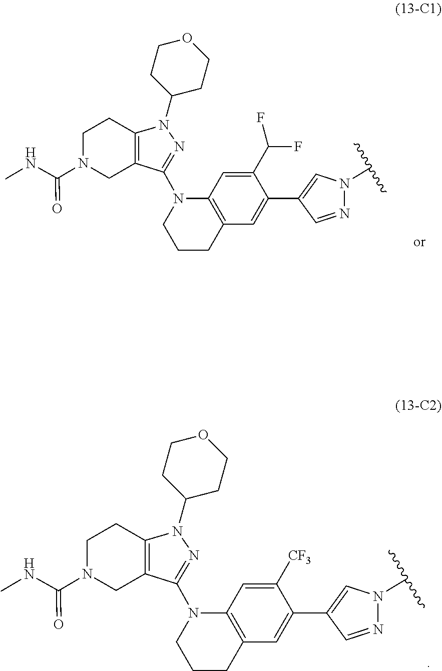

In some embodiments, the second terminus comprises the structure of Formula (13-D1) or Formula (13-D2), or a pharmaceutically acceptable salt thereof:

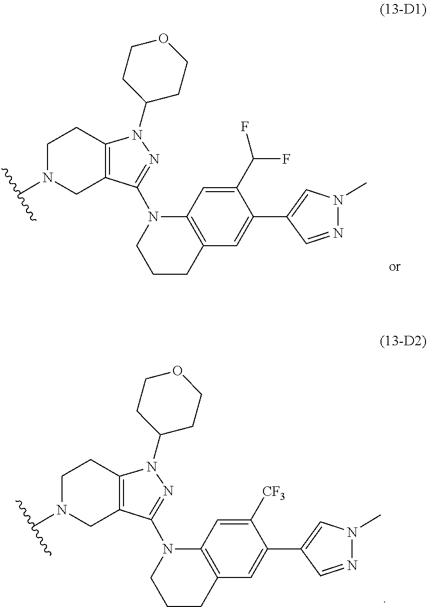

In some embodiments, the second terminus comprises the structure of Formula (14-A), or a pharmaceutically acceptable salt thereof:

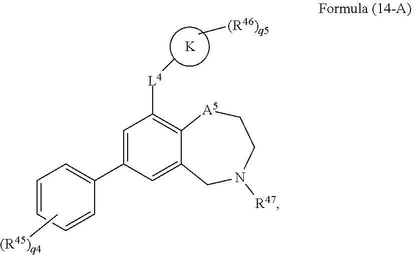

wherein:
Ring K is a 5 to 6-membered heterocycloalkyl;
A⁵ is absent, CH₂, —NH—, or —O—;
L⁴ is alkylene or heteroalkylene;
each R⁴⁵ is independently halogen, —OH, —CN, —NO₂, —NH₂, optionally substituted C₁-C₁₀ alkyl, optionally substituted C₁-C₁₀ haloalkyl, C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3 to 8-membered heterocycloalkyl;
each R⁴⁶ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ heteroalkyl, optionally substituted C₁-C₁₀ hydroxyalkyl, optionally substituted C₂-C₁₀ alkenyl, optionally substituted C₂-C₁₀ alkynyl, optionally substituted C₃-C₈ cycloalkyl, or optionally substituted 3 to 8-membered heterocycloalkyl;
R⁴⁷ is optionally substituted C₁-C₁₀ alkyl, —C(O)R^{47a}, or —C(O)—NR^{47a}R^{47b}, wherein
   R^{47a} and R^{47b} are each independently optionally substituted C₁-C₁₀ alkyl or optionally substituted C₃-C₈ cycloalkyl;
q₄ is 2-3; or
q₅ is 0-2; and
wherein the Formula (14-A) is connected to the linker through Ring K or through one of R⁴⁵.

In some embodiments, A⁵ is absent. In some embodiments, A⁵ is —NH— or —O—. In some embodiments, A⁵ is —NH—. In some embodiments, A⁵ is —O—.

In some embodiments, L⁴ is alkylene. In some embodiments, L⁴ is C₁-C₅ alkylene. In some embodiments, L⁴ is heteroalkylene. In some embodiments, L⁴ is C₁-C₄ heteroalkylene-. In some embodiments, L⁴ is —O—CH₂— or —O—CH₂CH₂—.

In some embodiments, each R⁴⁵ is independently halogen, —OH, —CN, —NO₂, —NH₂, optionally substituted C₁-C₁₀ alkyl, optionally substituted C₁-C₁₀ haloalkyl, C₁-C₁₀ hydroxyalkyl, optionally substituted C₂-C₁₀ alkenyl, or optionally substituted C₂-C₁₀ alkynyl. In some embodiments, each R⁴⁵ is independently optionally substituted C₁-C₁₀ alkyl or optionally substituted C₁-C₁₀ hydroxyalkyl. In some embodiments, each R⁴⁵ is independently C₁-C₁₀ hydroxyalkyl. In some embodiments, each R⁴⁵ is independently —OCH₃ or —OCH₂CH₃.

In some embodiments, each R⁴⁶ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ heteroalkyl, or optionally substituted C₁-C₁₀ hydroxyalkyl. In some embodiments, each R⁴⁶ is independently —OCH₃. In some embodiments, each R⁴⁶ is independently hydrogen.

In some embodiments, R⁴⁷ is optionally substituted C₁-C₁₀ alkyl. In some embodiments, R⁴⁷ is —C(O)R^{47a}. In some embodiments, R⁴⁷ is —C(O)CH₃ or —C(O)CH₂CH₃. In some embodiments, —C(O)—NR^{47a}R^{47b}.

In some embodiments, R^{47a} is optionally substituted C₁-C₁₀ alkyl. In some embodiments, R^{47a} is optionally substituted C₃-C₈ cycloalkyl.

In some embodiments, R^{47a} is optionally substituted C₁-C₁₀ alkyl. In some embodiments, R^{47a} is an optionally substituted C₃-C₈ cycloalkyl.

In some embodiments, Ring K is a 6-membered heterocycloalkyl.

In some embodiments, q₄ is 3. In some embodiments, q₄ is 2.

In some embodiments, q₅ is 2. In some embodiments, q₅ is 1. In some embodiments, q₅ is 0.

In some embodiments, Formula (14-A) is connected to the linker through Ring K. In some embodiments, Formula (14-A) is connected to the linker through one of R⁴⁵.

In some embodiments, the second terminus comprises the structure of Formula (14-B) or Formula (14-C), or a pharmaceutically acceptable salt thereof:

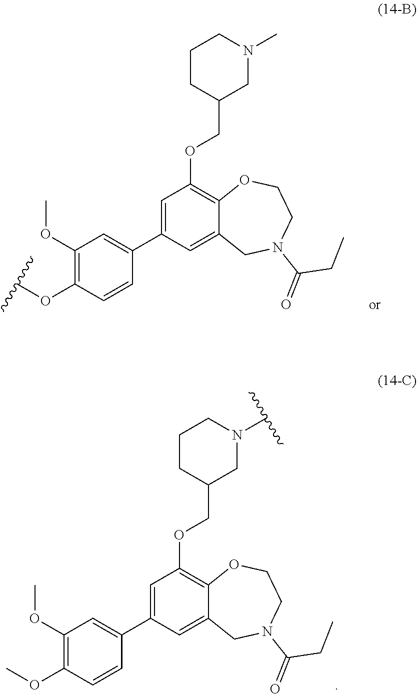

In some embodiments, the second terminus comprises the structure of Formula (15-A), or a pharmaceutically acceptable salt thereof:

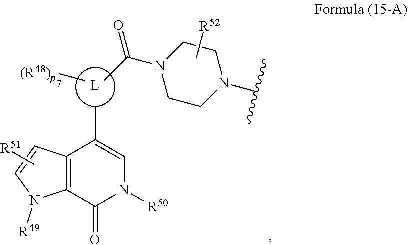

wherein:
Ring L is an aryl or heteroaryl;
each R⁴⁸ is hydrogen, halogen, —OH, —CN, —NO₂, —NH₂, C₁-C₁₀ alkyl, C₁-C₁₀ haloalkyl, or C₁-C₁₀ hydroxyalkyl;
R⁴⁹ and R⁵⁰ are each independently hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl;
R⁵¹ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, or optionally substituted C₂-C₁₀ alkynyl;
R⁵² is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl; and
p₇ is 1-4.

In some embodiments, Ring L is an aryl. In some embodiments, the aryl is phenyl. In some embodiments, Ring L is heteroaryl. In some embodiments, Ring L is a bicyclic heteroaryl comprising 1, 2, or 3 heteroatoms selected from N and O.

In some embodiments, wherein the second terminus comprises the structure of Formula (15-B), or a pharmaceutically acceptable salt thereof:

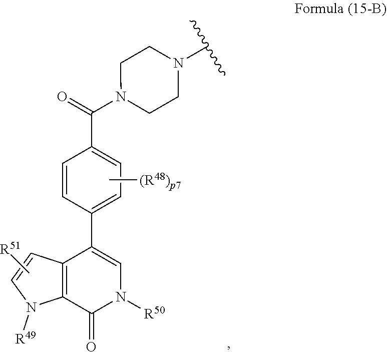

wherein:
each R⁴⁸ is hydrogen, halogen, —OH, —CN, —NO₂, —NH₂, C₁-C₁₀ alkyl, C₁-C₁₀ haloalkyl, or C₁-C₁₀ hydroxyalkyl;
R⁴⁹ and R⁵⁰ are each independently hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl;
R⁵¹ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, or optionally substituted C₂-C₁₀ alkynyl; and
p₇ is 1-4.

In some embodiments, wherein the second terminus comprises the structure of Formula (15-C), or a pharmaceutically acceptable salt thereof:

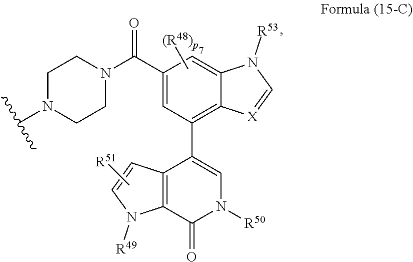

wherein:
X is CR⁴⁸ or N;
each R⁴⁸ is hydrogen, halogen, —OH, —CN, —NO₂, —NH₂, C₁-C₁₀ alkyl, C₁-C₁₀ haloalkyl, or C₁-C₁₀ hydroxyalkyl;
R⁴⁹ and R⁵⁰ are each independently hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl;
R⁵¹ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, or optionally substituted C₂-C₁₀ alkynyl;
R⁵³ is hydrogen or optionally substituted C₁-C₁₀ alkyl; and
p₇ is 1-3.

In some embodiments, each R⁴⁸ is hydrogen, halogen, —OH, —CN, —NO₂, —NH₂, C₁-C₁₀ alkyl, C₁-C₁₀ haloalkyl, or optionally substituted C₁-C₁₀ hydroxyalkyl.

In some embodiments, R⁴⁹ is hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl. In some embodiments, R⁴⁹ is an optionally substituted C₁-C₁₀ alkyl. In some embodiments, R⁴⁹ is methyl, ethyl, iso-propyl, or tert-butyl. In some embodiments, R⁴⁹ is hydrogen.

In some embodiments, R⁵⁰ is hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl. In some embodiments, R⁵⁰ is an optionally substituted C₁-C₁₀ alkyl or optionally substituted C₂-C₁₀ alkenyl. In some embodiments, R⁵⁰ is hydrogen.

In some embodiments, R⁵¹ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, or optionally substituted C₂-C₁₀ alkynyl.

In some embodiments, R⁵² is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl. In some embodiments, R⁵² is hydrogen.

In some embodiments, R⁵³ is hydrogen or optionally substituted C₁-C₁₀ alkyl. In some embodiments, R⁵³ is an optionally substituted C₁-C₁₀ alkyl. In some embodiments, R³ is methyl, ethyl, iso-propyl, or tert-butyl. In some embodiments, R⁵³ is hydrogen.

In some embodiments, p₇ is 4. In some embodiments, p₇ is 3. In some embodiments, p₇ is 2. In some embodiments, p₇ is 1.

In some embodiments, the second terminus comprises the structure of Formula (15-D1), (15-D2), or (15-D3), or a pharmaceutically acceptable salt thereof:

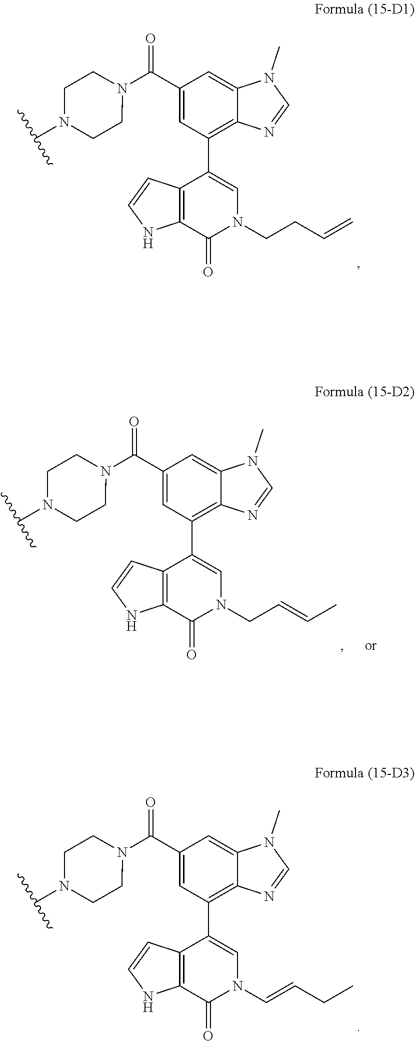 [SMILES: *N1CCN(C(=O)c2cc(-c3cn(CCC=C)c(=O)c4[nH]ccc34)c3ncn(C)c3c2)CC1 ; *N1CCN(C(=O)c2cc(-c3cn(C/C=C/C)c(=O)c4[nH]ccc34)c3ncn(C)c3c2)CC1 ; *N1CCN(C(=O)c2cc(-c3cn(/C=C/CC)c(=O)c4[nH]ccc34)c3ncn(C)c3c2)CC1]

In some embodiments, the second terminus comprises the structure of Formula (15-E1), (15-E2), or (15-E3), or a pharmaceutically acceptable salt thereof:

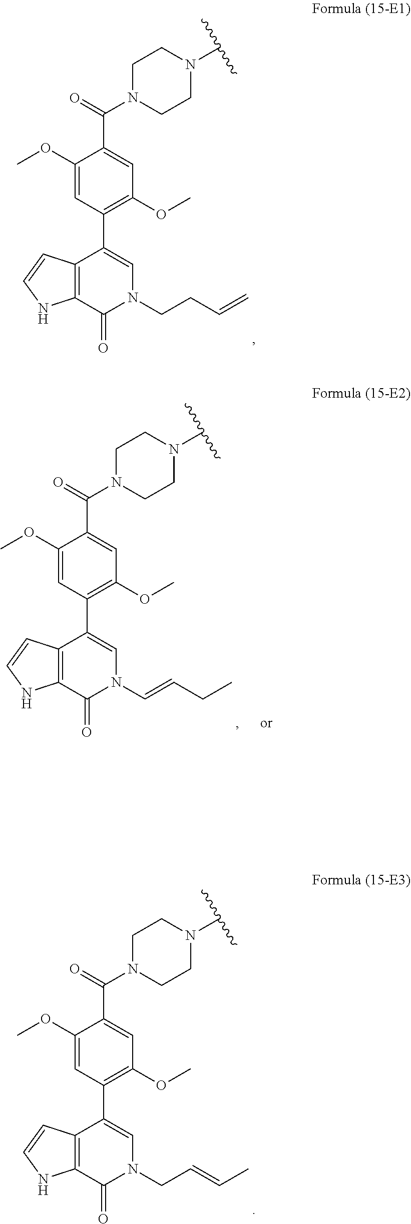

In some embodiments, the second terminus comprises the structure of Formula (16-A), or a pharmaceutically acceptable salt thereof:

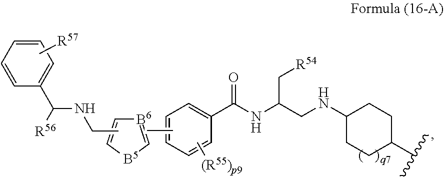

wherein:
B⁵ is —O—, —NH—, or S;
B⁶ is N or CH;
R⁵⁴ is optionally substituted aryl or optionally substituted heteroaryl;
each R⁵⁵ is independently halogen, —OH, —CN, —NO₂, —NH₂, optionally substituted C₁-C₁₀ alkyl, optionally substituted C₁-C₁₀ haloalkyl, optionally substituted C₁-C₁₀ heteroalkyl, or optionally substituted C₁-C₁₀ hydroxyalkyl;
R⁵⁶ is hydrogen, optionally substituted C₁-C₁₀ alkyl, optionally substituted C₁-C₁₀ haloalkyl, or optionally substituted C₁-C₁₀ hydroxyalkyl;
R⁵⁷ is halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl;
p₉ is 1-3; and
q₇ is 0-2.

In some embodiments, B⁵ is —O— or —S—. In some embodiments, B⁵ is —O—. In some embodiments, B⁵ is —S—.

In some embodiments, B⁶ is N. In some embodiments, B⁶ is CH.

In some embodiments, R⁵⁴ is an optionally substituted aryl. In some embodiments, R⁴ is phenyl optionally substituted with one or more halogen, —CN, —NH₂, —OH, C₁-C₁₀ alkyl, C₁-C₁₀ haloalkyl, or C₁-C₁₀ hydroxyalkyl.

In some embodiments, each R⁵⁵ is independently halogen, —OH, —CN, —NH₂, optionally substituted C₁-C₁₀ alkyl, optionally substituted C₁-C₁₀ haloalkyl, optionally substituted C₁-C₁₀ heteroalkyl, or optionally substituted C₁-C₁₀ hydroxyalkyl.

In some embodiments, R⁵⁶ is optionally substituted C₁-C₁₀ alkyl, optionally substituted C₁-C₁₀ haloalkyl, or optionally substituted C₁-C₁₀ hydroxyalkyl. In some embodiments, R⁶ is optionally substituted C₁-C₁₀ alkyl.

In some embodiments, R¹⁷ is halogen, —OH, —CN, —NO₂, —NH₂, or optionally substituted C₁-C₁₀ alkyl.

In some embodiments, p₉ is 3. In some embodiments, p₉ is 2. In some embodiments, p₉ is 1.

In some embodiments, q₇ is 2. In some embodiments, q₇ is 1. In some embodiments, q is 0.

In some embodiments, the second terminus comprises the structure of Formula (16-B), or a pharmaceutically acceptable salt thereof:

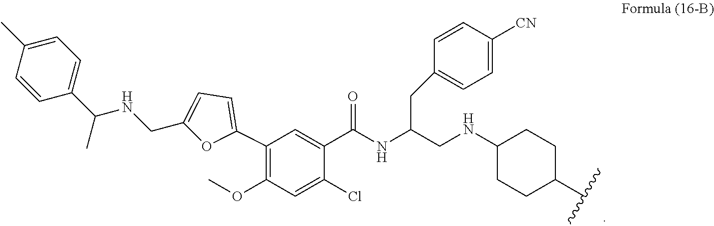 [SMILES: *C1CCC(NCC(Cc2ccc(C#N)cc2)NC(=O)c2cc(-c3ccc(CNC(C)c4ccc(C)cc4)o3)c(OC)cc2Cl)CC1]

In some embodiments, the second terminus comprises the structure of Formula (17-A), or a pharmaceutically acceptable salt thereof:

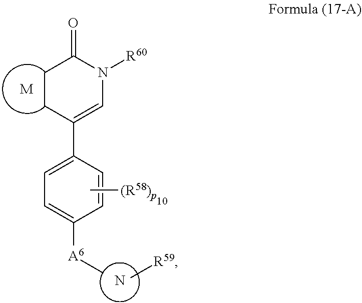

wherein:
Ring M is optionally substituted aryl or optionally substituted heteroaryl;
Ring N is absent or 4 to 8-membered heterocycloalkyl;
A⁶ is —O—, —NH—, or —CH₂—;
each R⁵⁸ is independently halogen, —OH, —CN, —NO₂, —NH₂, C₁-C₁₀ alkyl, C₁-C₁₀ haloalkyl, or C₁-C₁₀ hydroxyalkyl;
R⁵⁹ is hydrogen, —OH, —NH₂, C₁-C₁₀ alkyl, C₁-C₁₀ haloalkyl, C₁-C₁₀ hydroxyalkyl, or —NH—C₁-C₁₀ alkyl;
R⁶⁰ is hydrogen or optionally substituted C₁-C₁₀ alkyl; and
p₁₀ is 1-4; and
wherein Formula (17-A) is connected to the linker through R⁵⁹.

In some embodiments, Ring M is an aryl, optionally substituted with one or more halogen, CN, NH₂, OH, C₁-C₁₀ alkyl, C₁-C₁₀ haloalkyl, or C₁-C₁₀ hydroxyalkyl. In some embodiments, Ring M is phenyl. In some embodiments, Ring M is an optionally substituted 6-membered heteroaryl, optionally substituted with one or more halogen, CN, NH₂, OH, C₁-C₁₀ alkyl, C₁-C₁₀ haloalkyl, or C₁-C₁₀ hydroxyalkyl. In some embodiments, Ring M is an optionally substituted pyridine.

In some embodiments, Ring N is 4 to 8-membered heterocycloalkyl. In some embodiment, Ring N is a 4-membered heterocycloalkyl. In some embodiments, Ring N is a 5-membered heterocycloalkyl. In some embodiments, Ring N is a 6-membered heterocycloalkyl. In some embodiments, Ring N is absent.

In some embodiments, A⁶ is —O— or —NH—. In some embodiments, A⁶ is —CH₂—.

In some embodiments, each R⁵⁸ is independently —OH, —NH₂, C₁-C₁₀ alkyl, C₁-C₁₀ haloalkyl, or C₁-C₁₀ hydroxyalkyl. In some embodiments, each R⁵⁸ is independently C₁-C₁₀ alkyl, or C₁-C₁₀ hydroxyalkyl. In some embodiments, each R⁵⁸ is independently C₁-C₁₀ hydroxyalkyl.

In some embodiments, R⁵⁹ is —OH, —NH₂, C₁-C₁₀ hydroxyalkyl, or —NH—C₁-C₁₀ alkyl. In some embodiments, R⁵⁹ is hydrogen.

In some embodiments, R⁶⁰ is optionally substituted C₁-C₁₀ alkyl. In some embodiments, R⁶⁰ is methyl. In some embodiments, R⁶⁰ is hydrogen.

In some embodiments, p₁₀ is 3 or 4. In some embodiments, p₁₀ is 2. In some embodiments, p₁₀ is 1.

In some embodiments, the second terminus comprises the structure of Formula (17-B), or a pharmaceutically acceptable salt thereof:

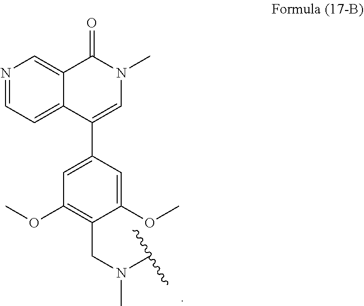 [SMILES: *N(C)Cc1c(OC)cc(-c2cn(C)c(=O)c3cnccc23)cc1OC]

In some embodiments, the second terminus comprises the structure of Formula (17-C), or a pharmaceutically acceptable salt thereof:

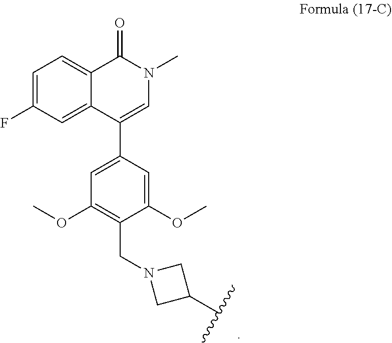 [SMILES: *C1CN(Cc2c(OC)cc(-c3cn(C)c(=O)c4ccc(F)cc34)cc2OC)C1]

In some embodiments, the second terminus is selected from the group consisting of:

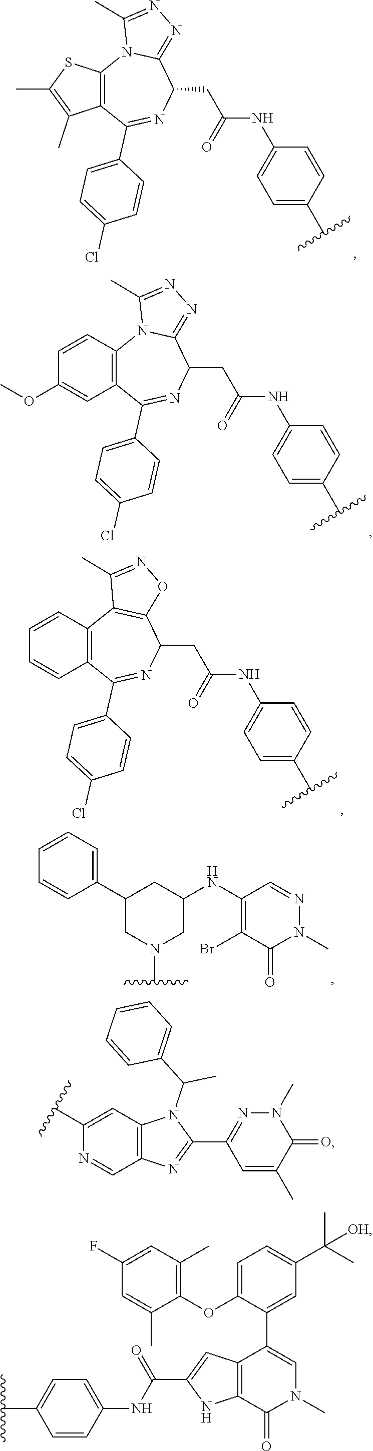

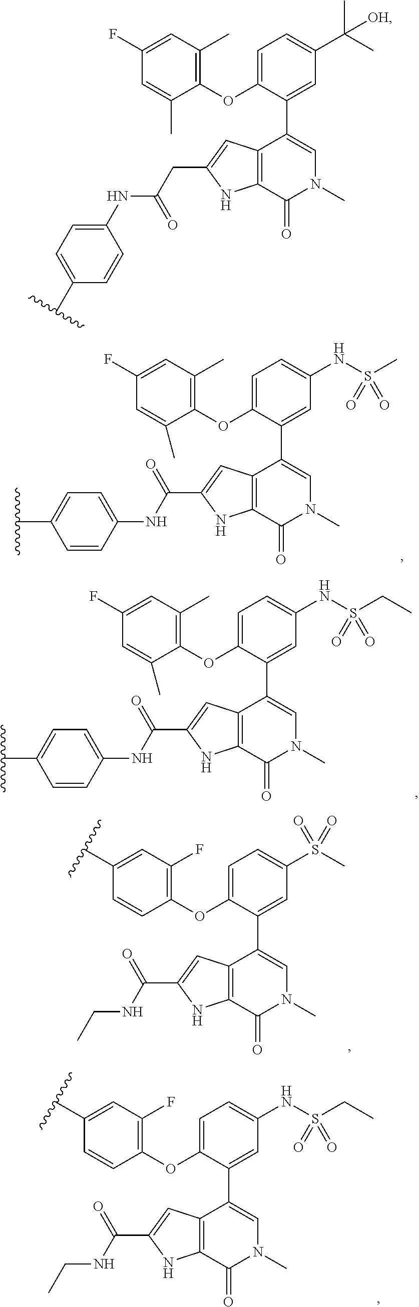

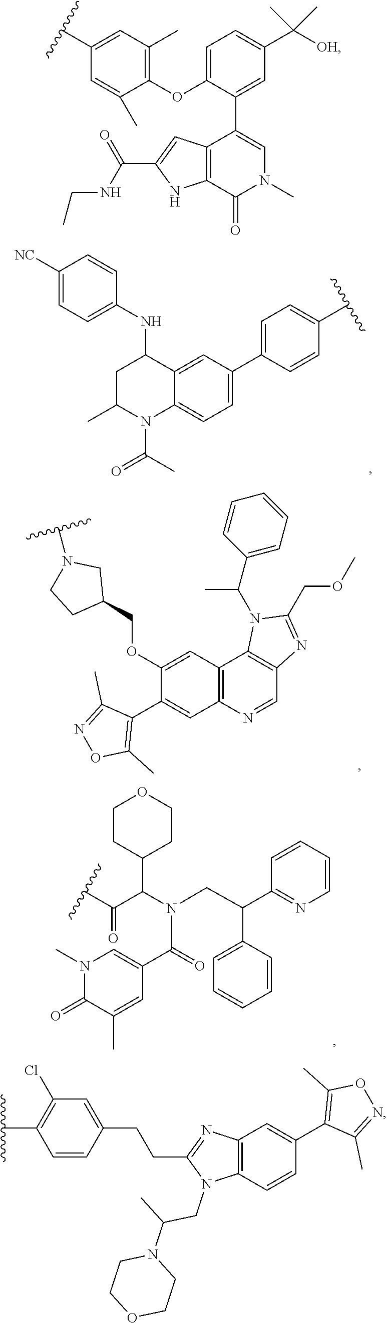

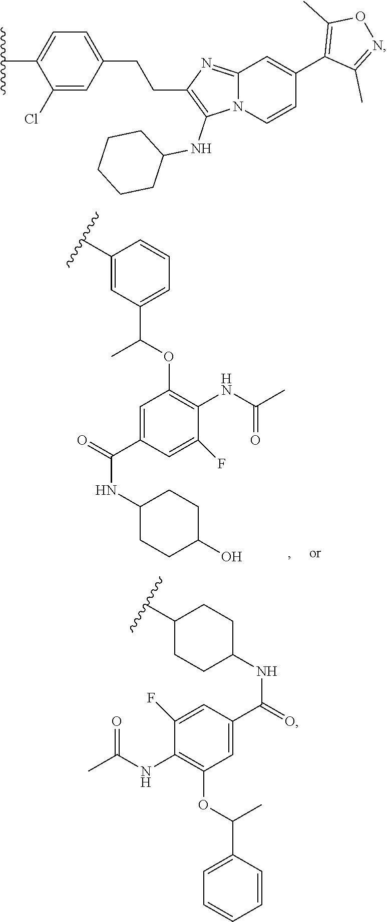 [SMILES: *c1ccc(CCc2nc3cc(-c4c(C)noc4C)ccn3c2NC2CCCCC2)cc1Cl ; *c1cccc(C(C)Oc2cc(C(=O)NC3CCC(O)CC3)cc(F)c2NC(C)=O)c1 ; *C1CCC(NC(=O)c2cc(F)c(NC(C)=O)c(OC(C)c3ccccc3)c2)CC1]

or a pharmaceutically acceptable salt thereof.

### Oligomeric Linker

The length of the oligomeric linker depends on the type of regulatory protein and also the target gene. In some embodiments, the linker has a length of less than about 50 Angstroms. In some embodiments, the linker has a length of about 20 to 30 Angstroms. In some embodiments, the oligomeric linker comprises between 5 and 50 chain atoms.

In some embodiments, the oligomeric linker comprises a multimer having 2 to 50 spacing moieties, wherein each spacing moiety is independently selected from the group consisting of —((CR^{1b}R^{1b})ₓ—O)_{y}—, —((CR^{1b}R^{1b})ₓ—NR^{1a})_{y}—, —((CR^{1b}R^{1b})ₓ—CH═CH—(CR^{1b}R^{1b})ₓ—O)_{y}—, optionally substituted C₁-C₁₂ alkyl, optionally substituted C₂-C₁₀ alkenyl, optionally substituted C₂-C₁₀ alkynyl, optionally substituted C₆-C₁₀ arylene, optionally substituted C₃-C₇ cycloalkylene, optionally substituted 5- to 10-membered heteroarylene, optionally substituted 4- to 10-membered heterocycloalkylene, amino acid residue, —O—, —C(O)NR^{1a}—, —NR^{1a}C(O)—, —C(O)—, —NR^{1a}—, —C(O)O—, —S—, —S(O)—, —S(O)₂—, —S(O)₂NR^{1a}—, —NR^{1a}S(O)₂—, and —P(O)OH—, and any combinations thereof, wherein
each x is independently 2-4;
each y is independently 1-10;
each R^{1a} is independently a hydrogen or optionally substituted C₁-C₆ alkyl; and each R^{1b} is independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl.

In some embodiments, the oligomeric linker comprises a multimer having 2 to 50 spacing moieties, wherein each spacing moiety is independently selected from the group consisting of optionally substituted C₁-C₁₂ alkyl, —((CH₂)ₓ—O)_{y}—, —((CH₂)ₓ—NH)_{y}—, —O—, —C(O)NH—, —NH—, and any combinations thereof.

In various aspects, the composition suitable for treating genetic diseases comprising a double stranded DNA binding moiety is suitable for topical administration. In some embodiments, the composition is suitable for administration to a human eye. In some embodiments, the composition further comprises an excipient. In some embodiments, the excipient comprises an isotonicity adjusting agent such as sodium chloride, a buffer, a stabilizer, an anti-oxidant, a viscosity enhancing agent, a solubilizer, a penetration enhancer, and/or a preservative. In some embodiments, the composition has a pH of about 5 to about 8. In some embodiments, the composition has a viscosity of from about 1 to about 50,000 cps at about 20° C.

In various aspects, administration of the composition suitable for treating genetic diseases comprising a double stranded DNA binding moiety to an eye results in minimal systemic exposure. In some embodiments, topical administration (e.g., ophthalmic administration) of the composition to an eye results in penetration of the double stranded DNA binding moiety to the internal cells and tissues of the eye. In some embodiments, at least about 10%, at least about 20%, at least about 30%, at least about 40%, at least about 50%, at least about 60%, at least about 70%, at least about 80%, at least about 85%, at least about 90%, at least about 95%, or at least about 99% of the ophthalmically administered double stranded DNA binding moiety penetrates the internal cells and tissues of the eye.

### Aqueous Dose-to-Dose Uniformity

Typical ophthalmic aqueous solutions, emulsions, or suspensions are packaged in eye drop bottles and administered as drops. For example, a single administration (i.e., a single dose) of an ophthalmic aqueous solution, emulsion, or suspension includes a single drop, two drops, three drops, or more into the eyes of the patient. In some embodiments, one dose of the ophthalmic aqueous solution, emulsion, or suspension described herein is one drop of the aqueous solution, emulsion, or suspension composition from the eye drop bottle.

In some cases, described herein include ophthalmic aqueous compositions which provide dose-to-dose uniform concentrations. In some instances, the dose-to-dose uniform concentration does not present significant variations of drug content from one dose to another. In some instances, the dose-to-dose uniform concentration does provide consistent drug content from one dose to another.

In some embodiments, the composition has a dose-to-dose ophthalmic agent concentration variation of less than 50%. In some embodiments, the composition has a dose-to-dose ophthalmic agent concentration variation of less than 40%. In some embodiments, the composition has a dose-to-dose ophthalmic agent concentration variation of less than 30%. In some embodiments, the composition has a dose-to-dose ophthalmic agent concentration variation of less than 20%. In some embodiments, the composition has a dose-to-dose ophthalmic agent concentration variation of less than 10%. In some embodiments, the composition has a dose-to-dose ophthalmic agent concentration variation of less than 5%.

In some embodiments, the dose-to-dose ophthalmic agent concentration variation is based on 10 consecutive doses. In some embodiments, the dose-to-dose ophthalmic agent concentration variation is based on 8 consecutive doses. In some embodiments, the dose-to-dose ophthalmic agent concentration variation is based on 5 consecutive doses. In some embodiments, the dose-to-dose ophthalmic agent concentration variation is based on 3 consecutive doses. In some embodiments, the dose-to-dose ophthalmic agent concentration variation is based on 2 consecutive doses.

A non-settling composition should not require shaking to disperse drug uniformly. A “no-shake” composition is potentially advantageous over compositions that require shaking for the simple reason that patients' shaking behavior is a major source of variability in the amount of drug dosed. It has been reported that patients often do not or forget to shake their compositions that require shaking before administering a dose, despite the instructions to shake that were clearly marked on the label. On the other hand, even for those patients who do shake the product, it is normally not possible to determine whether the shaking is adequate in intensity and/or duration to render the product uniform. In some embodiments, the ophthalmic gel compositions and ophthalmic ointment compositions described herein are “no-shake” formulations that maintained the dose-to-dose uniformity described herein. In some embodiments, the ophthalmic gel compositions and ophthalmic ointment compositions described herein require shaking to maintain the dose-to-dose uniformity described herein.

To evaluate the dose-to-dose uniformity, drop bottles or tubes containing the ophthalmic aqueous compositions, the ophthalmic gel compositions, or ophthalmic ointment compositions are stored upright for a minimum of 12 hours prior to the start of the test. To simulate the recommended dosing of these products, predetermined number of drops or strips are dispensed from each commercial bottles or tubes at predetermined time intervals for an extended period of time or until no product was left in the bottle or tube. All drops and strips are dispensed into tared glass vials, capped, and stored at room temperature until analysis. Concentrations of a double stranded DNA binding moiety in the expressed drops are determined using a reverse-phase HPLC method.

### Aqueous Viscosity

In some embodiments, the composition has a Brookfield RVDV viscosity of from about 1 to about 50,000 centipoise (cps) at about 20° C. and sheer rate of 1 s⁻¹. In some embodiments, the composition has a Brookfield RVDV viscosity of from about 100 to about 40,000 cps at about 20° C. and sheer rate of 1 s⁻¹. In some embodiments, the composition has a Brookfield RVDV viscosity of from about 500 to about 30,000 cps at about 20° C. and sheer rate of 1 s⁻¹. In some embodiments, the composition has a Brookfield RVDV viscosity of from about 1000 to about 20,000 cps at about 20° C. and sheer rate of 1 s⁻¹. In some embodiments, the composition has a Brookfield RVDV viscosity of from about 2000 to about 10,000 cps at about 20° C. and sheer rate of 1 s⁻¹. In some embodiments, the composition has a Brookfield RVDV viscosity of from about 4000 to about 8000 cps at about 20° C. and sheer rate of 1 s⁻¹.

In some embodiments, the aqueous composition contains a viscosity enhancing agent sufficient to provide a viscosity of between about 500 and 50,000 cps, between about 750 and 50,000 cps, between about 1000 and 50,000 cps, between about 1000 and 40,000 cps, between about 2000 and 30,000 cps, between about 3000 and 20,000 cps, between about 4000 and 10,000 cps, or between about 5000 and 8000 cps.

In some embodiments, the aqueous composition comprises a viscosity reducing agent. In some embodiments, the viscosity reducing agent comprises caffeine. In some embodiments, the aqueous composition comprises caffeine at a suitable concentration for achieving the desired viscosity. In some embodiments, the composition comprises caffeine at about 0.5%, about 1%, about 1.5%, about 2%, about 2.5%, about 3%, about 3.5%, about 4%, about 4.5%, about 5%, or about 10%.

In some embodiments, the compositions described herein are low viscosity compositions at body temperature. In some embodiments, low viscosity compositions contain from about 1% to about 10% of a viscosity enhancing agent (e.g., gelling components such as polyoxyethylene-polyoxypropylene copolymers). In some embodiments, low viscosity compositions contain from about 2% to about 10% of a viscosity enhancing agent (e.g., gelling components such as polyoxyethylene-polyoxypropylene copolymers). In some embodiments, low viscosity compositions contain from about 5% to about 10% of a viscosity enhancing agent (e.g., gelling components such as polyoxyethylene-polyoxypropylene copolymers). In some embodiments, low viscosity compositions are substantially free of a viscosity enhancing agent (e.g., gelling components such as polyoxyethylene-polyoxypropylene copolymers). In some embodiments, a low viscosity ophthalmic agent composition described herein provides an apparent viscosity of from about 100 cps to about 10,000 cps. In some embodiments, a low viscosity ophthalmic agent composition described herein provides an apparent viscosity of from about 500 cps to about 10,000 cps. In some embodiments, a low viscosity ophthalmic agent composition described herein provides an apparent viscosity of from about 1000 cps to about 10,000 cps.

### Osmolarity

In some embodiments, a composition disclosed herein is formulated in order to not disrupt the ionic balance of the eye. In some embodiments, a composition disclosed herein has an ionic balance that is the same as or substantially the same as the eye. In some embodiments, a composition disclosed herein does not disrupt the ionic balance of the eye.

As used herein, “practical osmolarity/osmolality” or “deliverable osmolarity/osmolality” means the osmolarity/osmolality of a composition as determined by measuring the osmolarity/osmolality of the ophthalmic agent and all excipients except the gelling and/or the thickening agent (e.g., polyoxyethylene-polyoxypropylene copolymers, carboxymethylcellulose or the like). The practical osmolarity of a composition disclosed herein is measured by a suitable method, e.g., a freezing point depression method as described in Viegas et. al., Int. J. Pharm., 1998, 160, 157-162. In some instances, the practical osmolarity of a composition disclosed herein is measured by vapor pressure osmometry (e.g., vapor pressure depression method) that allows for determination of the osmolarity of a composition at higher temperatures. In some instances, vapor pressure depression method allows for determination of the osmolarity of a composition comprising a gelling agent (e.g., a thermoreversible polymer) at a higher temperature wherein the gelling agent is in the form of a gel.

In some embodiments, the osmolarity at a target site of action (e.g., the eye) is about the same as the delivered osmolarity of a composition described herein. In some embodiments, a composition described herein has a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L, or about 250 mOsm/L to about 320 mOsm/L.

The practical osmolality of a composition disclosed herein is from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg, or from about 280 mOsm/kg to about 320 mOsm/kg. In some embodiments, a composition described herein has a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L.

In some embodiments, suitable tonicity adjusting agents include, but are not limited to any pharmaceutically acceptable sugar, salt or any combinations or mixtures thereof, such as, but not limited to dextrose, glycerin, mannitol, sorbitol, sodium chloride, and other electrolytes. In some instances, the tonicity adjusting agent is selected from sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In some embodiments, the compositions described herein include one or more salts in an amount required to bring osmolality of the composition into an acceptable range. Such salts include those having sodium, potassium, or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite, and ammonium sulfate.

### Sterility

In some embodiments, the compositions are sterilized. Included within the embodiments disclosed herein are means and processes for sterilization of a pharmaceutical composition disclosed herein for use in humans. The goal is to provide a safe pharmaceutical product, relatively free of infection causing microorganisms. The U. S. Food and Drug Administration has provided regulatory guidance in the publication “Guidance for Industry: Sterile Drug Products Produced by Aseptic Processing” available at: http://www.fda.gov/cder/guidance/5882fnl.htm, which is incorporated herein by reference in its entirety.

As used herein, sterilization means a process used to destroy or remove microorganisms that are present in a product or packaging. Any suitable method available for sterilization of objects and compositions is used. Available methods for the inactivation of microorganisms include, but are not limited to, the application of extreme heat, lethal chemicals, or gamma radiation. In some embodiments, a process for the preparation of an ophthalmic composition comprises subjecting the composition to a sterilization method selected from heat sterilization, chemical sterilization, radiation sterilization, or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

### Filtration Sterilization

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 μm. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C.: Health Industry Manufacturers Association, 1981) and involve challenging the membrane filter with a known quantity (ca. 10′/cm²) of unusually small microorganisms, such as Brevundimonas diminuta (ATCC 19146).

Pharmaceutical compositions are optionally sterilized by passing through membrane filters. Compositions comprising nanoparticles (U.S. Pat. No. 6,139,870) or multilamellar vesicles (Richard et al., International Journal of Pharmaceutics (2006), 312(1-2):144-50) are amenable to sterilization by filtration through 0.22 μm filters without destroying their organized structure.

In some embodiments, the methods disclosed herein comprise sterilizing the composition (or components thereof) by means of filtration sterilization. In ophthalmic gel compositions that include thermosetting polymers, filtration is carried out below (e.g., about 5° C.) the gel temperature (Tgel) of a composition described herein and with viscosity that allows for filtration in a reasonable time using a peristaltic pump (e.g., below a theoretical value of 100 cps).

Accordingly, provided herein are methods for sterilization of compositions provided herein that prevent degradation of polymeric components (e.g., thermosetting and/or other viscosity enhancing agents) and/or the ophthalmic agent during the process of sterilization. In some embodiments, degradation of the ophthalmic agent (e.g., a double stranded DNA binding moiety) is reduced or eliminated through the use of specific pH ranges for buffer components and specific proportions of viscosity enhancing agents in the compositions. In some embodiments, the choice of an appropriate viscosity enhancing agent or thermosetting polymer allows for sterilization of compositions described herein by filtration. In some embodiments, the use of an appropriate thermosetting polymer or other viscosity enhancing agents in combination with a specific pH range for the composition allows for high temperature sterilization of compositions described with substantially no degradation of the therapeutic agent or the polymeric excipients. An advantage of the methods of sterilization provided herein is that, in certain instances, the compositions are subjected to terminal sterilization via autoclaving without any loss of the ophthalmic agent and/or excipients and/or viscosity enhancing agents during the sterilization step and are rendered substantially free of microbes and/or pyrogens.

### Radiation Sterilization

One advantage of radiation sterilization is the ability to sterilize many types of products without heat degradation or other damage. The radiation commonly employed is beta radiation or, alternatively, gamma radiation from a ⁶⁰Co source. The penetrating ability of gamma radiation allows its use in the sterilization of many product types, including solutions, compositions, and heterogeneous mixtures. The germicidal effects of irradiation arise from the interaction of gamma radiation with biological macromolecules. This interaction generates charged species and free radicals. Subsequent chemical reactions, such as rearrangements and cross-linking processes, result in the loss of normal function for these biological macromolecules. The compositions described herein are also optionally sterilized using beta irradiation.

### Sterilization by Heat

Many methods are available for sterilization by the application of high heat. One method is through the use of a saturated steam autoclave. In this method, saturated steam at a temperature of at least 121° C. is allowed to contact the object to be sterilized. The transfer of heat is either directly to the microorganism, in the case of an object to be sterilized, or indirectly to the microorganism by heating the bulk of an aqueous solution to be sterilized. This method is widely practiced as it allows flexibility, safety, and economy in the sterilization process.

### Microorganisms

In some embodiments, the compositions are substantially free of microorganisms. Acceptable bioburden or sterility levels are based on applicable standards that define therapeutically acceptable compositions, including but not limited to United States Pharmacopeia Chapters <1111> et seq. For example, acceptable sterility (e.g., bioburden) levels include about 10 colony forming units (cfu) per gram of composition, about 50 cfu per gram of composition, about 100 cfu per gram of composition, about 500 cfu per gram of composition, or about 1000 cfu per gram of composition. In some embodiments, acceptable bioburden levels or sterility for compositions include less than 10 cfu/mL, less than 50 cfu/mL, less than 500 cfu/mL, or less than 1000 cfu/mL microbial agents. In addition, acceptable bioburden levels or sterility include the exclusion of specified objectionable microbiological agents. By way of example, specified objectionable microbiological agents include but are not limited to Escherichia coli (E. coli), Salmonella sp., Pseudomonas aeruginosa (P. aeruginosa), and/or other specific microbial agents.

An important component of the sterility assurance quality control, quality assurance, and validation process is the method of sterility testing. Sterility testing, by way of example only, is performed by two methods. The first is direct inoculation wherein a sample of the composition to be tested is added to growth medium and incubated for a period of time up to 21 days. Turbidity of the growth medium indicates contamination. Drawbacks to this method include the small sampling size of bulk materials which reduces sensitivity, and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the entire bulk product is sampled. The commercially available Millipore Steritest sterility testing system is optionally used for determinations by membrane filtration sterility testing. For the filtration testing of creams or ointments Steritest filter system No. TLHVSL210 is used. For the filtration testing of emulsions or viscous products Steritest filter system No. TLAREM210 or TDAREM210 is used. For the filtration testing of pre-filled syringes Steritest filter system No. TTHASY210 is used. For the filtration testing of material dispensed as an aerosol or foam Steritest filter system No. TTHVA210 is used. For the filtration testing of soluble powders in ampoules or vials Steritest filter system No. TTHADA210 or TTHADV210 is used.

Testing for E. coli and Salmonella includes the use of lactose broths incubated at 30-35° C. for 24-72 hours, incubation in MacConkey and/or EMB agars for 18-24 hours, and/or the use of Rappaport medium. Testing for the detection of P. aeruginosa includes the use of NAC agar. United States Pharmacopeia Chapter <62> further enumerates testing procedures for specified objectionable microorganisms.

In certain embodiments, the composition described herein has less than about 60 colony forming units (cfu), less than about 50 colony forming units, less than about 40 colony forming units, or less than about 30 colony forming units of microbial agents per gram of formulation. In certain embodiments, the compositions described herein are formulated to be isotonic with the eye.

### Endotoxins

An additional aspect of the sterilization process is the removal of by-products from the killing of microorganisms. The process of depyrogenation removes pyrogens from the sample. Pyrogens are endotoxins or exotoxins which induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins varies widely, the presence of endotoxins is expressed in “endotoxin units” (EU). One EU is equivalent to 100 picograms of E. coli LPS. In some cases, humans develop a response to as little as 5 EU/kg of body weight. The bioburden (e.g., microbial limit) and/or sterility (e.g., endotoxin level) is expressed in any units as recognized in the art. In certain embodiments, compositions described herein contain lower endotoxin levels (e.g., <4 EU/kg of body weight of a subject) when compared to conventionally acceptable endotoxin levels (e.g., 5 EU/kg of body weight of a subject). In some embodiments, the composition has less than about 5 EU/kg of body weight of a subject. In other embodiments, the composition has less than about 4 EU/kg of body weight of a subject. In additional embodiments, the composition has less than about 3 EU/kg of body weight of a subject. In additional embodiments, the composition has less than about 2 EU/kg of body weight of a subject.

In some embodiments, the composition has less than about 5 EU/kg of composition. In other embodiments, the composition has less than about 4 EU/kg of composition. In additional embodiments, the composition has less than about 3 EU/kg of composition. In some embodiments, the composition has less than about 2 EU/kg of composition. In other embodiments, the composition has less than about 1 EU/kg of composition. In additional embodiments, the composition has less than about 0.2 EU/kg of composition. In certain embodiments, composition described herein contain from about 1 to about 5 EU/mL of composition. In certain embodiments, composition described herein contain from about 2 to about 5 EU/mL of composition, from about 3 to about 5 EU/mL of composition, or from about 4 to about 5 EU/mL of composition.

In certain embodiments, compositions described herein contain lower endotoxin levels (e.g., <0.5 EU/mL of composition) when compared to conventionally acceptable endotoxin levels (e.g., 0.5 EU/mL of composition). In some embodiments, the composition has less than about 0.5 EU/mL of composition. In other embodiments, the composition has less than about 0.4 EU/mL of composition. In additional embodiments, the composition has less than about 0.2 EU/mL of composition.

Pyrogen detection, by way of example only, is performed by several methods. Suitable tests for sterility include tests described in United States Pharmacopoeia (USP)<71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, MD, 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the composition is subject to depyrogenation. In a further embodiment, the process for the manufacture of the composition comprises testing the composition for pyrogenicity. In certain embodiments, the compositions described herein are substantially free of pyrogens.

### Ophthalmic Gel Composition

Gels have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

In some embodiments, gels are also classified as being hydrophobic or hydrophilic. In certain embodiments, the base of a non-limiting example of a hydrophobic gel includes a liquid paraffin with polyethylene or fatty oils gelled with colloidal silica, or aluminum or zinc soaps. In contrast, the base of a non-limiting example of a hydrophilic gel includes water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions disclosed herein is pseudo plastic, plastic, thixotropic, or dilatant.

In some embodiments, the composition is an ophthalmic gel, and wherein the ophthalmically acceptable carrier comprises water and at least one viscosity-enhancing agent. In some embodiments, the viscosity-enhancing agent is selected from cellulose-based polymers, polyoxyethylene-polyoxypropylene triblock copolymers, dextran-based polymers, polyvinyl alcohol, dextrin, polyvinylpyrrolidone, polyalkylene glycols, chitosan, collagen, gelatin, hyaluronic acid, or combinations thereof.

In some embodiments, the composition comprises a viscosity reducing agent. In some embodiments, the viscosity reducing agent comprises caffeine. In some embodiments, the composition comprises caffeine at a suitable concentration for achieving the desired viscosity. In some embodiments, the composition comprises caffeine at about 0.5%, about 1%, about 1.5%, about 2%, about 2.5%, about 3%, about 3.5%, about 4%, about 4.5%, about 5%, or about 10%.

In some embodiments, the ophthalmic gel composition described herein is a semi-solid or is in a gelled state before it is topically administered (e.g., at room temperature). For example, suitable viscosity-enhancing agents for such gels include by way of example only, gelling agents and suspending agents. In one embodiment, the enhanced viscosity composition does not include a buffer. In other embodiments, the enhanced viscosity composition includes a pharmaceutically acceptable buffer. Sodium chloride or other tonicity agents are optionally used to adjust tonicity, if necessary.

By way of example only, the ophthalmically acceptable viscosity agent includes hydroxypropyl methylcellulose, hydroxyethyl cellulose, polyvinylpyrrolidone, carboxymethyl cellulose, polyvinyl alcohol, sodium chondroitin sulfate, or sodium hyaluronate. Other viscosity enhancing agents compatible with the targeted ocular site include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthan gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethylcellulose (CMC), silicon dioxide, polyvinylpyrrolidone (PVP: povidone), Splenda® (dextrose, maltodextrin and sucralose), or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the ophthalmic agents disclosed herein acts as a controlled release composition, restricting the diffusion of the ophthalmic agents from the composition. Moreover, the combination of carboxymethylated chitosan and alginate is optionally used to assist in increasing the permeability of the agents in the eye.

In some embodiments, provided herein is an enhanced viscosity composition comprising from about 0.1 mM and about 100 mM of an ophthalmic agent, a pharmaceutically acceptable viscosity agent, and water for injection, the concentration of the viscosity agent in the water being sufficient to provide an enhanced viscosity composition with a final viscosity from about 100 to about 100,000 cps. In certain embodiments, the viscosity of the gel is in the range from about 100 to about 50,000 cps, about 100 cps to about 1,000 cps, about 500 cps to about 1500 cps, about 1000 cps to about 3000 cps, about 2000 cps to about 8,000 cps, about 4,000 cps to about 50,000 cps, about 10,000 cps to about 500,000 cps, or about 15,000 cps to about 1,000,000 cps. In other embodiments, when an even more viscous medium is desired, the biocompatible gel comprises at least about 35%, at least about 45%, at least about 55%, at least about 65%, at least about 70%, at least about 75%, or at least about 80% or so by weight of the ophthalmic agent. In highly concentrated samples, the biocompatible enhanced viscosity composition comprises at least about 25%, at least about 35%, at least about 45%, at least about 55%, at least about 65%, at least about 75%, at least about 85%, at least about 90%, or at least about 95% or more by weight of the ophthalmic agent.

In one embodiment, the pharmaceutically acceptable enhanced viscosity ophthalmically acceptable composition comprises at least one ophthalmic agent and at least one gelling agent. Suitable gelling agents for use in preparation of the gel composition include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel compositions presented herein.

In some embodiments, the ophthalmic gel composition described herein is an in situ gel composition. In some instances, the in situ gel composition is based on increased pre-corneal residence time of the composition which improves ocular bioavailability, corneal mucoadhesion, lysosomal interaction and ionic gelation, improved corneal absorption, thermal gelation, or a combination thereof. In some instances, the in situ gel formulation is activated by pH, temperature, ion, UV, or solvent exchange.

In some instances, the ophthalmic gel composition comprises a double stranded DNA binding moiety and one or more gelling agents. In some instances, the gelling agent includes, but is not limited to, poloxamer (e.g. Poloxamer 407), tetronics, ethyl (hydroxyethyl) cellulose, cellulose acetate phthalate (CAP), carbopol (e.g. Carbopol 1342P NF, Carbopol 980 NF), alginates (e.g. low acetyl gellan gum (Gelrite®)), gellan, hyaluronic acid, pluronics (e.g. Pluronic F-127), chitosan, polyvinyl alcohol (PVA), polyvinylpyrrolidone (PVP), dextran, hydroxy propyl methyl cellulose (HPMC), hydroxyethylcellulose (HEC), methylcellulose (MC), thiolated xyloglucan, polymethacrylic acid (PMMA), polyethylene glycol (PEG), pseudolatexes, xyloglucans, or combinations thereof.

In some instances, the in situ gel composition further comprises a permeation enhancer. In some instances, the permeation enhancer includes surfactants (e.g., non-ionic surfactants), benzalkonium chloride, EDTA, surface-active heteroglycosides, calcium chelators, hydroxyl propyl beta cyclodextrin (HP beta CD), bile salts, and the like.

In some embodiments, other gel composition are useful depending upon the particular ophthalmic agent, other pharmaceutical agent or excipients/additives used, and as such are considered to fall within the scope of the present disclosure. For example, other commercially available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the ophthalmic agent compositions described herein. In some embodiments, ophthalmically acceptable gels include, but are not limited to, alginate hydrogels SAF®-Gel (ConvaTec, Princeton, N.J.), Duoderm® Hydroactive Gel (ConvaTec), Nu-gel® (Johnson & Johnson Medical, Arlington, Tex.); Carrasyn®(V) Acemannan Hydrogel (Carrington Laboratories, Inc., Irving, Tex.); glycerin gels Elta® Hydrogel (Swiss-American Products, Inc., Dallas, Tex.), and K—Y® Sterile (Johnson & Johnson). In further embodiments, biodegradable biocompatible gels also represent compounds present in ophthalmically acceptable composition disclosed and described herein.

In some embodiments, the viscosity-enhancing agent is a cellulose-based polymer selected from cellulose gum, alkylcellulose, hydroxyl-alkyl cellulose, hydroxyl-alkyl alkylcellulose, carboxy-alkyl cellulose, or combinations thereof. In some embodiments, the viscosity-enhancing agent is hydroxyl-alkyl alkylcellulose. In some embodiments, the viscosity-enhancing agent is hydroxypropyl methylcellulose.

In certain embodiments, the enhanced viscosity composition is characterized by a phase transition between room temperature and body temperature (including an individual with a serious fever, e.g., up to about 42° C.). In some embodiments, the phase transition occurs at 1° C. below body temperature, at 2° C. below body temperature, at 3° C. below body temperature, at 4° C. below body temperature, at 6° C. below body temperature, at 8° C. below body temperature, or at 10° C. below body temperature. In some embodiments, the phase transition occurs at about 15° C. below body temperature, at about 20° C. below body temperature or at about 25° C. below body temperature. In specific embodiments, the gelation temperature (Tgel) of a composition described herein is about 20° C., about 25° C., or about 30° C. In certain embodiments, the gelation temperature (Tgel) of a composition described herein is about 35° C., or about 40° C. Included within the definition of body temperature is the body temperature of a healthy individual, or an unhealthy individual, including an individual with a fever (up to −42° C.). In some embodiments, the pharmaceutical compositions described herein are liquids at about room temperature and are administered at or about room temperature.

Copolymers polyoxypropylene and polyoxyethylene (e.g., polyoxyethylene-polyoxypropylene triblock copolymers) form thermosetting gels when incorporated into aqueous solutions. These polymers have the ability to change from the liquid state to the gel state at temperatures close to body temperature, therefore allowing useful compositions that are applied to the targeted ocular site. The liquid state-to-gel state phase transition is dependent on the polymer concentration and the ingredients in the solution.

In some embodiments, the amount of thermosetting polymer in any composition described herein is about 10%, about 15%, about 20%, about 25%, about 30%, about 35%, or about 40% of the total weight of the composition. In some embodiments, the amount of thermosetting polymer in any composition described herein is about 10%, about 11%, about 12%, about 13%, about 14%, about 15%, about 16%, about 17%, about 18%, about 19%, about 20%, about 21%, about 22%, about 23%, about 24%, or about 25% of the total weight of the composition. In some embodiments, the amount of thermosetting polymer (e.g., Poloxamer 407) in any composition described herein is about 7.5% of the total weight of the composition. In some embodiments, the amount of thermosetting polymer (e.g., Poloxamer 407) in any composition described herein is about 10% of the total weight of the composition. In some embodiments, the amount of thermosetting polymer (e.g., Poloxamer 407) in any composition described herein is about 11% of the total weight of the composition. In some embodiments, the amount of thermosetting polymer (e.g., Poloxamer 407) in any composition described herein is about 12% of the total weight of the composition. In some embodiments, the amount of thermosetting polymer (e.g., Poloxamer 407) in any composition described herein is about 13% of the total weight of the composition. In some embodiments, the amount of thermosetting polymer (e.g., Poloxamer 407) in any composition described herein is about 14% of the total weight of the composition. In some embodiments, the amount of thermosetting polymer (e.g., Poloxamer 407) in any composition described herein is about 15% of the total weight of the composition. In some embodiments, the amount of thermosetting polymer (e.g., Poloxamer 407) in any composition described herein is about 16% of the total weight of the composition. In some embodiments, the amount of thermosetting polymer (e.g., Poloxamer 407) in any composition described herein is about 17% of the total weight of the composition. In some embodiments, the amount of thermosetting polymer (e.g., Poloxamer 407) in any composition described herein is about 18% of the total weight of the composition. In some embodiments, the amount of thermosetting polymer (e.g., Poloxamer 407) in any composition described herein is about 19% of the total weight of the composition. In some embodiments, the amount of thermosetting polymer (e.g., Poloxamer 407) in any composition described herein is about 20% of the total weight of the composition. In some embodiments, the amount of thermosetting polymer (e.g., Poloxamer 407) in any composition described herein is about 21% of the total weight of the composition. In some embodiments, the amount of thermosetting polymer (e.g., Poloxamer 407) in any composition described herein is about 22% of the total weight of the composition. In some embodiments, the amount of thermosetting polymer (e.g., Poloxamer 407) in any composition described herein is about 23% of the total weight of the composition. In some embodiments, the amount of thermosetting polymer (e.g., Poloxamer 407) in any composition described herein is about 24% of the total weight of the composition. In some embodiments, the amount of thermosetting polymer (e.g., Poloxamer 407) in any composition described herein is about 25% of the total weight of the composition. In some embodiments, the amount of thickening agent (e.g., a gelling agent) in any composition described herein is about 1%, about 5%, about 10%, or about 15% of the total weight of the composition. In some embodiments, the amount of thickening agent (e.g., a gelling agent) in any composition described herein is about 0.5%, about 1%, about 1.5%, about 2%, about 2.5%, about 3%, about 3.5%, about 4%, about 4.5%, or about 5% of the total weight of the composition.

In an alternative embodiment, the thermogel is a PEG-PLGA-PEG triblock copolymer (Jeong et al, Nature (1997), 388:860-2; Jeong et al, J. Control. Release (2000), 63:155-63; Jeong et al, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting copolymers are soluble in water and form a free-flowing liquid at room temperature but form a hydrogel at body temperature. A commercially available PEG-PLGA-PEG triblock copolymer is RESOMER RGP t50106 manufactured by Boehringer Ingelheim. This material is composed of a PLGA copolymer of 50:50 poly(DL-lactide-co-glycolide) and is 10% w/w of PEG and has a molecular weight of about 6000.

Additional biodegradable thermoplastic polyesters include AtriGel® (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or a combination thereof. In one embodiment, the biodegradable thermoplastic polyester is 50/50 poly(DL-lactide-co-glycolide) having a carboxy terminal group; is present in about 30 wt. % to about 40 wt. % of the composition; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the composition; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermosetting gels dissolve more completely at reduced temperatures, methods of solubilization include adding the required amount of polymer to the amount of water to be used at reduced temperatures. Generally, after wetting the polymer by shaking, the mixture is capped and placed in a cold chamber or in a thermostatic container at about 0-10° C. in order to dissolve the polymer. The mixture is stirred or shaken to bring about a more rapid dissolution of the thermosetting gel polymer. The ophthalmic agent and various additives such as buffers, salts, and preservatives are subsequently added and dissolved. In some instances, the pharmaceutically agent is suspended if it is insoluble in water. The pH is modulated by the addition of appropriate buffering agents.

### Ophthalmic Ointment Composition

An ointment is a homogeneous, viscous, semi-solid preparation, most commonly a greasy, thick oil (e.g., oil 80%-water 20%) with a high viscosity, intended for external application to the skin or mucous membranes. Ointments have a water number that defines the maximum amount of water that it contains. They are used as emollients or for the application of active ingredients to the skin for protective, therapeutic, or prophylactic purposes and where a degree of occlusion is desired. Ointments are used topically on a variety of body surfaces. These include the skin and the mucous membranes of the eye (an eye ointment), vulva, anus, and nose.

The vehicle of an ointment is known as the ointment base. The choice of a base depends upon the clinical indication for the ointment. The different types of ointment bases are hydrocarbon bases, e.g., hard paraffin, soft paraffin, microcrystalline wax and ceresine; absorption bases, e.g., wool fat, beeswax; water soluble bases, e.g., macrogols 200, 300, 400; emulsifying bases, e.g., emulsifying wax, cetrimide; vegetable oils, e.g., olive oil, coconut oil, sesame oil, almond oil, and peanut oil.

Ointments are formulated using hydrophobic, hydrophilic, or water-emulsifying bases to provide preparations that are immiscible, miscible, or emulsifiable with skin secretions. In some embodiments, they are also derived from hydrocarbon (fatty), absorption, water-removable, or water-soluble bases. The active agents are dispersed in the base, and later they get divided after the drug penetration into the target sites (e.g., membranes, skins, etc.).

The present disclosure recognizes that it is sometimes difficult to incorporate into the ointment a drug of low concentration with sufficient dose-to-dose uniformity for effectively treating a disorder or disease. In some embodiments, poly(ethylene-glycols), polyethoxylated castor oils (Cremophor®EL), alcohols having 12 to 20 carbon atoms, or a mixture of two or more of said components are effective excipients for dispersing and/or dissolving effective amounts of ophthalmic drugs. The resulting ointments are excellently tolerated by the skin and by ocular tissue.

The present disclosure further recognizes that ophthalmic drugs, such as a double stranded DNA binding moiety, incorporated in the ointment compositions described herein target the choroid and/or retina in a patient when the compositions are topically administered to the ocular surface, in particular to the sclera of said patient. In some embodiments, an ophthalmic ointment composition includes an ophthalmic drug, an ointment base, and an agent for dispersing and/or dissolving said drug in the ointment base, selected from a poly(ethylene-glycol), a polyethoxylated castor oil, an alcohol having 12 to 20 carbon atoms and a mixture of two or more of said components.

In some embodiments, the ointment bases include ophthalmically acceptable oil and fat bases, such as natural wax e.g., white and yellow bees wax, carnauba wax, wool wax (wool fat), purified lanolin, anhydrous lanolin; petroleum wax e.g., hard paraffin, microcrystalline wax; hydrocarbons e.g., liquid paraffin, white and yellow soft paraffin, white petrolatum, yellow petrolatum; or combinations thereof.

The above-mentioned oil and fat bases are described in more detail, for instance, in the British Pharmacopoeia, Edition 2001, or the European Pharmacopoeia, 3rd Edition.

In some embodiments, the ointment base is present in amounts of about 50 to about 95, preferably of 70 to 90% by weight based on the total weight of the composition.

A preferred ointment base comprises a combination of one or more of one or more natural waxes like those indicated above, preferably wool wax (wool fat), and one or more hydrocarbons like those indicated above, preferably a soft paraffin or a petrolatum, more preferably in combination with liquid paraffin.

A special embodiment of the aforementioned ointment base comprises e.g., 5 to 17 parts by weight of wool fat, and 50 to 65 parts by weight of white petrolatum as well as 20 to 30 parts by weight of liquid paraffin.

In some embodiments, the agent for dispersing and/or dissolving the ophthalmic drug in the ointment base is selected from a poly(ethylene-glycol), a polyethoxylated castor oil, an alcohol having 12 to 20 carbon atoms and a mixture of two or more of said components. The agent is preferably used in amounts of 1 to 20 percent, more preferably 1 to 10 percent by weight of the entire semisolid composition.

Alcohols having 12 to 20 carbon atoms include particularly stearyl alcohol (C₁₈H₃₇OH), cetyl alcohol (C₁₆H₃₃OH) and mixtures thereof. Preferred are so-called cetostearyl alcohols, mixtures of solid alcohols substantially consisting of stearyl and cetyl alcohol and preferably comprising not less than 40 percent by weight of stearyl alcohol and a sum of stearyl alcohol and cetyl alcohol amounting to at least 90 percent by weight, and compositions comprising not less than 80 percent by weight of cetostearyl alcohol and an emulsifier, in particular sodium cetostearyl sulfate and/or sodium lauryl sulfate, preferably in amounts not less than 7 percent by weight of emulsifier.

Polyethoxylated castor oils are reaction products of natural or hydrogenated castor oils and ethylene glycol. In some instances, such products are obtained in known manner, e.g., by reaction of a natural or hydrogenated castor oil or fractions thereof with ethylene oxide, e.g., in a molar ratio from about 1:30 to about 1:60, with optional removal of free polyethylene glycol components from the product, e.g., in accordance with the methods disclosed in German Auslegeschriften 1,182,388 and 1,518,819. Especially suitable and preferred is a product commercially available under the trade name Cremophor®EL having a molecular weight (by steam osmometry)=ca. 1630, a saponification no.=ca. 65-70, an acid no.=ca. 2, an iodine no.=ca. 28-32 and an nD 25=ca.1.471. Also suitable for use in this category is, for instance, Nikkol®HCO-60, a reaction product of hydrogenated castor oil and ethylene oxide exhibiting the following characteristics: acid no.=ca. 0.3; saponification no.=ca. 47.4; hydroxy value=ca. 42.5. pH (5%)=ca. 4.6; Color APHA=ca. 40; m.p.=ca. 36.0° C.; Freezing point=ca. 32.4° C.; H2O content (%, KF)=ca. 0.03.

Poly(ethylene-glycols) are used in some embodiments as the agent for dispersing and/or dissolving the ophthalmic drug in the ointment base according to the present disclosure. Suitable poly(ethylene-glycol)s are typically mixtures of polymeric compounds of the general formula H—(OCH₂—CH₂)ₙOH, wherein the index n typically range from 4 to 230 and the mean molecular weight typically ranges from about 200 to about 10000. In some embodiments, n is a number from about 6 to about 22 and the mean molecular weight between about 300 and about 1000. In some embodiments, n ranges from about 6 to about 13 and the mean molecular weight from about 300 to about 600. In some embodiments, n has a value of about 8.5 to about 9 and the relative molecular weight is about 400. Suitable poly(ethylene-glycols) are readily available commercially, for example poly(ethylene-glycols) having a mean molecular weight of about 200, 300, 400, 600, 1000, 1500, 2000, 3000, 4000, 6000, 8000, and 10000.

In some embodiments, the poly(ethylene-glycols), in particular the types described in the foregoing paragraph, are used in amounts of 1 to 10 percent, or 1 to 5 percent by weight of the entire semisolid composition.

In some embodiments, the compositions comprise an agent for dispersing and/or dissolving of the drug in the ointment base which is selected from a poly(ethylene-glycol), a polyethoxylated castor oil and preferably a mixture of said components.

### Gel/Ointment Viscosity

In some embodiments, the composition has a Brookfield RVDV viscosity of from about 10,000 to about 300,000 cps at about 20° C. and sheer rate of 1 s⁻¹. In some embodiments, the composition has a Brookfield RVDV viscosity of from about 15,000 to about 200,000 cps at about 20° C. and sheer rate of 1 s⁻¹. In some embodiments, the composition has a Brookfield RVDV viscosity of from about 50,000 to about 150,000 cps at about 20° C. and sheer rate of 1 s⁻¹. In some embodiments, the composition has a Brookfield RVDV viscosity of from about 70,000 to about 130,000 cps at about 20° C. and sheer rate of 1 s⁻¹. In some embodiments, the composition has a Brookfield RVDV viscosity of from about 90,000 to about 110,000 cps at about 20° C. and sheer rate of 1 s⁻¹.

In some embodiments, the ophthalmic gel composition contains a viscosity enhancing agent sufficient to provide a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. In some embodiments, the ophthalmic gel composition contains a viscosity enhancing agent sufficient to provide a viscosity of between about 50,0000 and 1,000,000 centipoise.

In some embodiments, the compositions described herein are low viscosity compositions at body temperature. In some embodiments, low viscosity compositions contain from about 1% to about 10% of a viscosity enhancing agent (e.g., gelling components such as polyoxyethylene-polyoxypropylene copolymers). In some embodiments, low viscosity compositions contain from about 2% to about 10% of a viscosity enhancing agent (e.g., gelling components such as polyoxyethylene-polyoxypropylene copolymers). In some embodiments, low viscosity compositions contain from about 5% to about 10% of a viscosity enhancing agent (e.g., gelling components such as polyoxyethylene-polyoxypropylene copolymers). In some embodiments, low viscosity compositions are substantially free of a viscosity enhancing agent (e.g., gelling components such as polyoxyethylene-polyoxypropylene copolymers). In some embodiments, a low viscosity ophthalmic agent composition described herein provides an apparent viscosity of from about 100 cps to about 10,000 cps. In some embodiments, a low viscosity ophthalmic agent composition described herein provides an apparent viscosity of from about 500 cps to about 10,000 cps. In some embodiments, a low viscosity ophthalmic agent composition described herein provides an apparent viscosity of from about 1000 cps to about 10,000 cps.

In some embodiments, the compositions described herein are viscous compositions at body temperature. In some embodiments, viscous compositions contain from about 10% to about 25% of a viscosity enhancing agent (e.g., gelling components such as polyoxyethylene-polyoxypropylene copolymers). In some embodiments, the viscous compositions contain from about 14% to about 22% of a viscosity enhancing agent (e.g., gelling components such as polyoxyethylene-polyoxypropylene copolymers). In some embodiments, the viscous compositions contain from about 15% to about 21% of a viscosity enhancing agent (e.g., gelling components such as polyoxyethylene-polyoxypropylene copolymers). In some embodiments, a viscous composition described herein provides an apparent viscosity of from about 100,000 cps to about 1,000,000 cps. In some embodiments, a viscous composition described herein provides an apparent viscosity of from about 150,000 cps to about 500,000 cps. In some embodiments, a viscous composition described herein provides an apparent viscosity of from about 250,000 cps to about 500,000 cps. In some of such embodiments, a viscous composition is a liquid at room temperature and gels at about between room temperature and body temperature (including an individual with a serious fever, e.g., up to about 42° C.). In some embodiments, a viscous composition is administered as monotherapy for treatment of an ophthalmic disease or condition described herein.

In some embodiments, the viscosity of the gel compositions presented herein is measured by any means described. For example, in some embodiments, an LVDV-II+CP Cone Plate Viscometer and a Cone Spindle CPE-40 is used to calculate the viscosity of the gel composition described herein. In other embodiments, a Brookfield (spindle and cup) viscometer is used to calculate the viscosity of the gel composition described herein. In some embodiments, the viscosity ranges referred to herein are measured at room temperature. In other embodiments, the viscosity ranges referred to herein are measured at body temperature (e.g., at the average body temperature of a healthy human).

### Gel/Ointment Dose-to-Dose Uniformity

Typical ophthalmic gels are packaged in eye drop bottles and administered as drops. For example, a single administration (i.e., a single dose) of an ophthalmic gel includes a single drop, two drops, three drops, or more into the eyes of the patient. Furthermore, typical ophthalmic ointments are packaged in tubes or other squeezable containers with a dispensing nozzle through which strips of the ointment are delivered. For example, a single administration (i.e., a single dose) of an ophthalmic ointment includes a single strip, or multiple strips into the eyes of the patient. In some embodiments, one dose of the ophthalmic gel described herein is one drop of the gel composition from the eye drop bottle. In some embodiments, one dose of the ophthalmic ointment is one strip of the ointment composition dispensed through the nozzle of a dispersing tube.

In some cases, described herein, the ophthalmic gel compositions provide dose-to-dose uniform concentrations. In some instances, the dose-to-dose uniform concentration does not present significant variations of drug content from one dose to another. In some instances, the dose-to-dose uniform concentration does provide consistent drug content from one dose to another.

In some cases, described herein, the ophthalmic ointment compositions provide dose-to-dose uniform concentrations. In some instances, the dose-to-dose uniform concentration does not present significant variations of drug content from one dose to another. In some instances, the dose-to-dose uniform concentration does provide consistent drug content from one dose to another.

In some embodiments, the composition has a dose-to-dose ophthalmic agent concentration variation of less than 50%. In some embodiments, the composition has a dose-to-dose ophthalmic agent concentration variation of less than 40%. In some embodiments, the composition has a dose-to-dose ophthalmic agent concentration variation of less than 30%. In some embodiments, the composition has a dose-to-dose ophthalmic agent concentration variation of less than 20%. In some embodiments, the composition has a dose-to-dose ophthalmic agent concentration variation of less than 10%. In some embodiments, the composition has a dose-to-dose ophthalmic agent concentration variation of less than 5%.

In some embodiments, the dose-to-dose ophthalmic agent concentration variation is based on 10 consecutive doses. In some embodiments, the dose-to-dose ophthalmic agent concentration variation is based on 8 consecutive doses. In some embodiments, the dose-to-dose ophthalmic agent concentration variation is based on 5 consecutive doses. In some embodiments, the dose-to-dose ophthalmic agent concentration variation is based on 3 consecutive doses. In some embodiments, the dose-to-dose ophthalmic agent concentration variation is based on 2 consecutive doses.

A non-settling composition should not require shaking to disperse drug uniformly. A “no-shake” composition is potentially advantageous over compositions that require shaking for the simple reason that patients' shaking behavior is a major source of variability in the amount of drug dosed. It has been reported that patients often do not or forget to shake their compositions that requires shaking before administering a dose, despite the instructions to shake that were clearly marked on the label. On the other hand, even for those patients who do shake the product, it is normally not possible to determine whether the shaking is adequate in intensity and/or duration to render the product uniform. In some embodiments, the ophthalmic gel compositions and ophthalmic ointment compositions described herein are “no-shake” compositions that maintained the dose-to-dose uniformity described herein.

To evaluate the dose-to-dose uniformity, drop bottles or tubes containing the ophthalmic aqueous compositions, the ophthalmic gel compositions, or ophthalmic ointment compositions are stored upright for a minimum of 12 hours prior to the start of the test. To simulate the recommended dosing of these products, predetermined number of drops or strips are dispensed from each commercial bottles or tubes at predetermined time intervals for an extended period of time or until no product was left in the bottle or tube. All drops and strips are dispensed into tared glass vials, capped, and stored at room temperature until analysis. Concentrations of a double stranded DNA binding moiety in the expressed drops are determined using a reverse-phase HPLC method.

### Methods of Treatment

In an aspect, provided herein are methods of treating an eye disorder in an individual in need thereof. In some embodiments, the method comprises administering a double stranded deoxyribonucleic acid (DNA) binding moiety to the eye. In some embodiments, systemic exposure to the double stranded DNA binding moiety is minimal. In some embodiments, the double stranded DNA binding moiety binds to a repeat sequence. In some embodiments, the repeat sequence does not comprise multiple copies of GAA. In some embodiments, the repeat sequence comprises a repeat of at least four nucleotides. In some embodiments, the repeat sequence comprises multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG. In some embodiments, the repeat sequence comprises multiple copies of CTG or CAG. In some embodiments, the repeat sequence is not a triplet repeat. In some embodiments, the double stranded DNA binding moiety is suitable for treating a genetic disease. In some embodiments, the genetic disease is not Friedreich's ataxia. In some embodiments, the double stranded DNA binding moiety is a polymer that does not comprise nucleotides or amino acids. In some embodiments, the double stranded DNA binding moiety binds to a minor groove of the double stranded DNA. In some embodiments, the double stranded DNA binding moiety is not an antibiotic.

In aspects of methods of treatment provided herein, the double stranded DNA binding moiety binds to a sequence, such as a repeat sequence, that is adjacent to or within a gene. In some embodiments, the double stranded DNA binding moiety modulates expression of the gene. In some embodiments the double stranded DNA binding moiety increases expression of the gene. In some embodiments, the double stranded DNA binding moiety decreases expression of the gene. In some embodiments, the gene is Transcription factor 4 (TCF4).

In aspects of methods of treatment provided herein, the double stranded DNA binding moiety binds to double stranded DNA non-covalently. In some embodiments, the double stranded DNA binding moiety binds to double stranded DNA using electrostatic forces, such as hydrogen bonds, or Van der Waals forces. In some embodiments, the double stranded DNA binding moiety does not comprise a polynucleotide. In some embodiments, the double stranded DNA binding moiety does not comprise a polypeptide.

In aspects of methods of treatment provided herein, the double stranded DNA binding moiety has a molecular weight of less than about 15 kDa, less than about 12.5 kDa, less than about 10 kDa, less than about 9.5 kDa, less than about 9 kDa, less than about 8.5 kDa, less than about 8 kDa, less than about 7.5 kDa, less than about 7 kDa, less than about 6.5 kDa, less than about 6 kDa, less than about 5.5 kDa, less than about 5 kDa, less than about 4.5 kDa, less than about 4 kDa, less than about 3.5 kDa, less than about 3 kDa, less than about 2.5 kDa, less than about 2 kDa, or less than about 1.5 kDa. In some embodiments, the double stranded DNA binding moiety has a molecular weight of less than about 10 kDa, about 7.5 kDa, about 5 kDa, or about 2.5 kDa.

In some embodiments, the double stranded DNA binding moiety comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker.

In aspects of methods of treatment provided herein, the individual is a mammal. In some embodiments, the individual is a mouse, a rat, a cat, a dog, a rabbit, a horse, a cow, a pig, or a human. In some embodiments, the individual is a human.

In aspects of methods of treatment provided herein, the double stranded DNA binding moiety is formulated in a composition comprising an excipient suitable for administration to the eye. In some embodiments, the excipient comprises an isotonicity adjusting agent such as sodium chloride, a buffer, a stabilizer, an anti-oxidant, a viscosity enhancing agent, a solubilizer, a penetration enhancer, and/or a preservative. In some embodiments, the composition has a pH of about 5 to about 8. In some embodiments, the composition has a viscosity of from about 1 to about 50,000 cps at about 20° C.

In various aspects, administration of the composition to an eye results in minimal systemic exposure. In some embodiments, topical administration (e.g., ophthalmic administration) of the composition to an eye results in penetration of the double stranded DNA binding moiety to the internal cells and tissues of the eye. In some embodiments, at least about 10%, at least about 20%, at least about 30%, at least about 40%, at least about 50%, at least about 60%, at least about 70%, at least about 80%, at least about 85%, at least about 90%, at least about 95%, or at least about 99% of the ophthalmically administered double stranded DNA binding moiety penetrates the internal cells and tissues of the eye.

In another aspect, provided herein are methods of treating an eye disorder in an individual in need thereof, the methods comprising administering a double stranded DNA binding moiety, wherein the double stranded DNA binding moiety is a polymer that does not comprise nucleotides or amino acids, and wherein the composition is suitable for administration to a human eye. In some embodiments, the double stranded DNA binding moiety does not bind to a repeat sequence comprising multiple copies of GAA. In some embodiments, the double stranded DNA binding moiety binds to a repeat sequence comprising multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG. In some embodiments, the repeat sequence comprises multiple copies of CTG or CAG. In some embodiments, the repeat sequence comprises at least four nucleotides. In some embodiments, the repeat sequence is not a triplet repeat.

In aspects of methods of treatment with a double stranded DNA binding moiety that does not comprise nucleotides or amino acids, the disorder is a genetic disease. In some embodiments, the disorder is not Friedreich's ataxia. In some embodiments, the double stranded DNA binding moiety is not an antibiotic. In some embodiments, the double stranded DNA binding moiety binds to a minor groove of the double stranded DNA.

In aspects of methods of treatment provided herein, the double stranded DNA binding moiety that does not comprise nucleotides or amino acids binds to a sequence, such as a repeat sequence, that is adjacent to or within a gene. In some embodiments, the double stranded DNA binding moiety modulates expression of the gene. In some embodiments the double stranded DNA binding moiety increases expression of the gene. In some embodiments, the double stranded DNA binding moiety decreases expression of the gene. In some embodiments, the gene is Transcription factor 4 (TCF4).

In aspects of methods of treatment provided herein, the double stranded DNA binding moiety that does not comprise nucleotides or amino acids binds to double stranded DNA non-covalently. In some embodiments, the double stranded DNA binding moiety binds to double stranded DNA using electrostatic forces, such as hydrogen bonds, or Van der Waals forces. In some embodiments, the double stranded DNA binding moiety does not comprise a polynucleotide. In some embodiments, the double stranded DNA binding moiety does not comprise a polypeptide.

In aspects of methods of treatment provided herein, the double stranded DNA binding moiety that does not comprise nucleotides or amino acids has a molecular weight of less than about 15 kDa, less than about 12.5 kDa, less than about 10 kDa, less than about 9.5 kDa, less than about 9 kDa, less than about 8.5 kDa, less than about 8 kDa, less than about 7.5 kDa, less than about 7 kDa, less than about 6.5 kDa, less than about 6 kDa, less than about 5.5 kDa, less than about 5 kDa, less than about 4.5 kDa, less than about 4 kDa, less than about 3.5 kDa, less than about 3 kDa, less than about 2.5 kDa, less than about 2 kDa, or less than about 1.5 kDa. In some embodiments, the double stranded DNA binding moiety has a molecular weight of less than about 10 kDa, about 7.5 kDa, about 5 kDa, or about 2.5 kDa.

In some embodiments, the double stranded DNA binding moiety that does not comprise nucleotides or amino acids comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker.

In aspects of methods of treatment where the double stranded DNA binding moiety does not comprise nucleotides or amino acids provided herein, the individual is a mammal. In some embodiments, the individual is a mouse, a rat, a cat, a dog, a rabbit, a horse, a cow, a pig, or a human. In some embodiments, the individual is a human.

In aspects of methods of treatment where the double stranded DNA binding moiety does not comprise nucleotides or amino acids provided herein, the double stranded DNA binding moiety is formulated in a composition comprising an excipient suitable for administration to the eye. In some embodiments, the excipient comprises an isotonicity adjusting agent such as sodium chloride, a buffer, a stabilizer, an anti-oxidant, a viscosity enhancing agent, a solubilizer, a penetration enhancer, and/or a preservative. In some embodiments, the composition has a pH of about 5 to about 8. In some embodiments, the composition has a viscosity of from about 1 to about 50,000 cps at about 20° C.

In various aspects, administration of the composition where the double stranded DNA binding moiety does not comprise nucleotides or amino acids to an eye results in minimal systemic exposure. In some embodiments, topical administration (e.g., ophthalmic administration) of the composition to an eye results in penetration of the double stranded DNA binding moiety to the internal cells and tissues of the eye. In some embodiments, at least about 10%, at least about 20%, at least about 30%, at least about 40%, at least about 50%, at least about 60%, at least about 70%, at least about 80%, at least about 85%, at least about 90%, at least about 95%, or at least about 99% of the ophthalmically administered double stranded DNA binding moiety penetrates the internal cells and tissues of the eye.

In a further aspect, provided herein are methods of treating an eye disorder in an individual in need thereof, the methods comprising administering a double stranded DNA binding moiety, wherein the eye disorder is a genetic disease. In some embodiments, the genetic disease is not Friedreich's ataxia. In some embodiments, the double stranded DNA binding moiety is not an antibiotic. In some embodiments, the double stranded DNA binding moiety does not comprise nucleotides or amino acids.

In some embodiments of methods of treating an eye disorder, wherein the eye disorder is a genetic disease, the double stranded DNA binding moiety does not bind to a repeat sequence comprising multiple copies of GAA. In some embodiments, the double stranded DNA binding moiety binds to a repeat sequence comprising multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG. In some embodiments, the repeat sequence comprises multiple copies of CTG or CAG. In some embodiments, the repeat sequence comprises at least four nucleotides. In some embodiments, the repeat sequence is not a triplet repeat. In some embodiments, the double stranded DNA binding moiety binds to a minor groove of the double stranded DNA.

In aspects of methods of treatment of an eye disorder that is a genetic disease provided herein, the double stranded DNA binding moiety binds to a sequence, such as a repeat sequence, that is adjacent to or within a gene. In some embodiments, the double stranded DNA binding moiety modulates expression of the gene. In some embodiments the double stranded DNA binding moiety increases expression of the gene. In some embodiments, the double stranded DNA binding moiety decreases expression of the gene. In some embodiments, the gene is Transcription factor 4 (TCF4).

In aspects of methods of treatment of an eye disorder that is a genetic disease provided herein, the double stranded DNA binding moiety binds to double stranded DNA non-covalently. In some embodiments, the double stranded DNA binding moiety binds to double stranded DNA using electrostatic forces, such as hydrogen bonds, or Van der Waals forces. In some embodiments, the double stranded DNA binding moiety does not comprise a polynucleotide. In some embodiments, the double stranded DNA binding moiety does not comprise a polypeptide.

In aspects of methods of treatment of an eye disorder that is a genetic disease provided herein, the double stranded DNA binding moiety has a molecular weight of less than about 15 kDa, less than about 12.5 kDa, less than about 10 kDa, less than about 9.5 kDa, less than about 9 kDa, less than about 8.5 kDa, less than about 8 kDa, less than about 7.5 kDa, less than about 7 kDa, less than about 6.5 kDa, less than about 6 kDa, less than about 5.5 kDa, less than about 5 kDa, less than about 4.5 kDa, less than about 4 kDa, less than about 3.5 kDa, less than about 3 kDa, less than about 2.5 kDa, less than about 2 kDa, or less than about 1.5 kDa. In some embodiments, the double stranded DNA binding moiety has a molecular weight of less than about 10 kDa, about 7.5 kDa, about 5 kDa, or about 2.5 kDa.

In some embodiments of methods of treatment of an eye disorder that is a genetic disease, the double stranded DNA binding moiety comprises a polyamide. In some embodiments, the double stranded DNA binding moiety further comprises a transcriptional modulator binding moiety. In some embodiments, the double stranded DNA binding moiety is connected to the transcriptional modulator binding moiety by a linker.

In aspects of methods of treatment of an eye disorder that is a genetic disease provided herein, the individual is a mammal. In some embodiments, the individual is a mouse, a rat, a cat, a dog, a rabbit, a horse, a cow, a pig, or a human. In some embodiments, the individual is a human.

In aspects of methods of treatment of an eye disorder that is a genetic disease provided herein, the double stranded DNA binding moiety is formulated in a composition comprising an excipient suitable for administration to the eye. In some embodiments, the excipient comprises an isotonicity adjusting agent such as sodium chloride, a buffer, a stabilizer, an anti-oxidant, a viscosity enhancing agent, a solubilizer, a penetration enhancer, and/or a preservative. In some embodiments, the composition has a pH of about 5 to about 8. In some embodiments, the composition has a viscosity of from about 1 to about 50,000 cps at about 20° C.

In various aspects of methods of treatment of an eye disorder that is a genetic disease, administration of the composition to an eye results in minimal systemic exposure. In some embodiments, topical administration (e.g., ophthalmic administration) of the composition to an eye results in penetration of the double stranded DNA binding moiety to the internal cells and tissues of the eye. In some embodiments, at least about 10%, at least about 20%, at least about 30%, at least about 40%, at least about 50%, at least about 60%, at least about 70%, at least about 80%, at least about 85%, at least about 90%, at least about 95%, or at least about 99% of the ophthalmically administered double stranded DNA binding moiety penetrates the internal cells and tissues of the eye.

In various aspects of methods of treatment provided herein, tissue biodistribution may be evaluated in single and multidose ophthalmic administration studies conducted in experimental models such as rats, rabbits and dogs. In some cases, following ophthalmic administration, levels of the double stranded DNA binding agent in ocular tissues consistently demonstrate a gradient of exposure with highest concentrations of the double stranded DNA binding agent measured in the outermost ocular structures closest to the site of application, decreasing to the inner most corneal endothelial layer, a therapeutic target tissue for the double stranded DNA binding agent.

In some cases, the double stranded DNA binding agent is detected in corneal endothelium up to 24 hours, for example 2-24 hours, 6-24 hours, 12-24 hours, 18-24 hours, 2-18 hours, 6-18 hours, 12-18 hours, 2-12 hours, 6-12 hours, or 2-6 hours, following about 14 days of repeat doses, for example 1-28 days, 7-28 days, 14-28 days, 21-28 days, 1-21 days, 7-21 days, 14-21 days, 1-14 days, 7-14 days, or 1-7 days (once or twice per day) at concentrations that equal or exceed 3 to 20 nM, for example at least 3 nM, at least 6 nM, at least 9 nM, at least 12 nM, at least 15 nM, at least 18 nM, at least 20 nM or more, which are concentrations predicted to reduce symptoms, such as quantity of nuclear foci and instances of spliceopathy, in corneal endothelial cells (CECs) of some diseased individuals. In one embodiment, following a final ophthalmic dose, for example within 10 minutes, 20 minutes, 30 minutes, 40 minutes, 50 minutes, 1 hour, 1.2 hours, 1.4 hours, 1.6 hours, 1.8 hours, and the like, endothelial concentrations up to 10-fold, 20 fold, 30-fold, 40-fold, 50-fold, 60-fold, 80-fold, 100-fold, or 120-fold greater than the lowest or highest predicted efficacious concentrations. In some cases, efficacious concentrations are achieved when the double stranded DNA binding agent is administered once daily over several days, weeks, or months using clinically relevant dosing paradigms. In some cases, efficacious concentrations are achieved when the double stranded DNA binding agent is administered twice daily in over several days, weeks, or months using clinically relevant dosing paradigms. In some cases, results from distribution studies utilizing a water formulation, such as an acidified water formulation, demonstrate that ophthalmic dosing results in concentrations of the double stranded DNA binding agent in the target endothelium that exceed concentrations predicted to be biologically relevant for about 24 hours post-dose.

In various aspects of methods of treatment provided herein, a water formulation, such as an acidified water formulation, optionally containing mannitol, such as 1-10% mannitol, for example 2-10%, 2-8%, 2-6%, 2-4%, 4-10%, 4-8%, 4-6%, 6-10%, 6-8%, or 8-10% is selected as an ophthalmic formulation for the double stranded DNA binding agent. In some cases, the double stranded DNA binding agent formulated in this manner is well-tolerated locally after daily administration for about 7 days, such as 1-14 days, 1-10 days, 1-8 days, 1-6 days, 1-4 days, 1-2 days, 2-14 days, 2-10 days, 2-8 days, 2-6 days, 2-4 days, 4-14 days, 4-10 days, 4-8 days, 4-6 days, 6-14 days, 6-10 days, 6-8 days, 8-14 days, 8-10 days, or 10-14 days, utilizing an ophthalmic dose regimen (e.g., four 50 μL drops twice daily per eye in both eyes, for example 1, 2, 3, 4, 5, or 6 drops, of 10 μL, 20 μL, 30 μL, 40 μL, 50 μL, 60 μL, 70 μL, 80 μL, 90 μL, or 100 μL, once, twice, or three times per day) and concentrations (0.6% and 1%, such as 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9% or 2%) employed in about 4-week studies, such as 1-4 weeks, 2-4 weeks, or 1-2 weeks GLP studies in rabbits and dogs. In some cases, ocular target tissue levels are equivalent to those observed in earlier studies using acidified water alone. In some cases, the highest exposures of the double stranded DNA binding agent are observed in the corneal endothelium within the initial hour following topical instillation, such as 20 minutes, 40 minutes, 1 hour following treatment. Subsequent lowering of levels of the double stranded DNA binding agent in the corneal endothelium over the next 2-24 hours, such as 2 hours, 4 hours, 6 hours, 8 hours, 10 hours, 12 hours, 14 hours, 16 hours, 18 hours, 20 hours, 22 hours or 24 hours, to about 3-40 nM, such as 3 nM, 6 nM, 8 nM, 10 nM, 12 nM, 14 nM, 16 nM, 18 nM, 20 nM, 24 nM, 28 nM, 32 nM, 36 nM, or 40 nM, is suggestive of diffusion and/or transport across the cornea. In some cases, the double stranded DNA binding agent is detected in aqueous humor up to 12-48 hours following the last dose, for example 12 hours, 16 hours, 20 hours, 24 hours, 28 hours, 32 hours, 36 hours, 40 hours, 44 hours, or 48 hours. In some cases, the full volume of aqueous humor is replenished approximately every 1-4 hours, such as 1 hour, 2 hours, 3 hours, or 4 hours, in research animals such as rabbits, therefore, measurable levels of the double stranded DNA binding agent in the aqueous humor at later time points is suggestive of sustained release of the double stranded DNA binding agent from the cornea into posterior eye compartments.

TABLE 1
Example Double Stranded DNA Binding Agent Structures

| # | Structure |
|---|---|
| 201 | 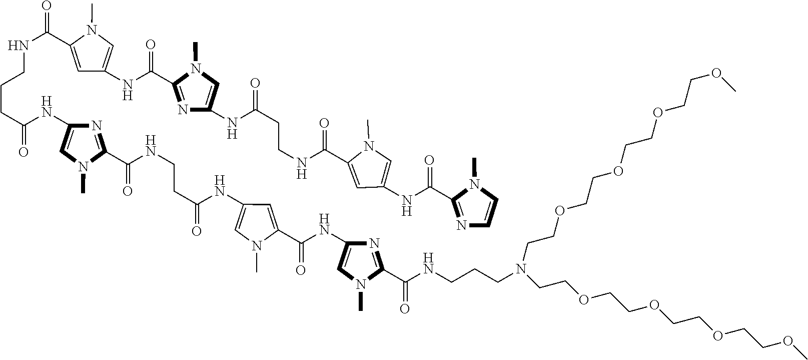 |
| 202 | 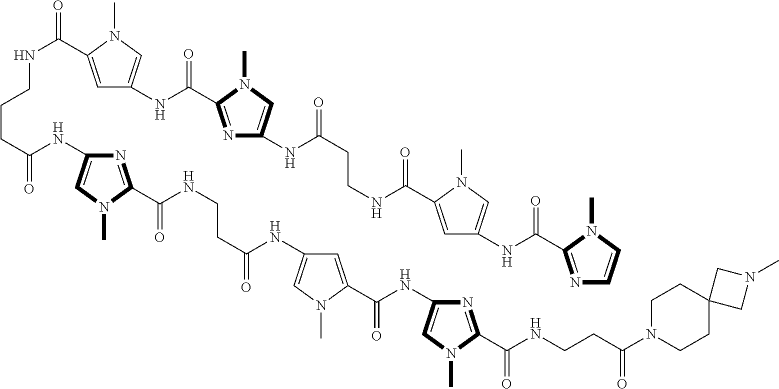 |
| 203 | 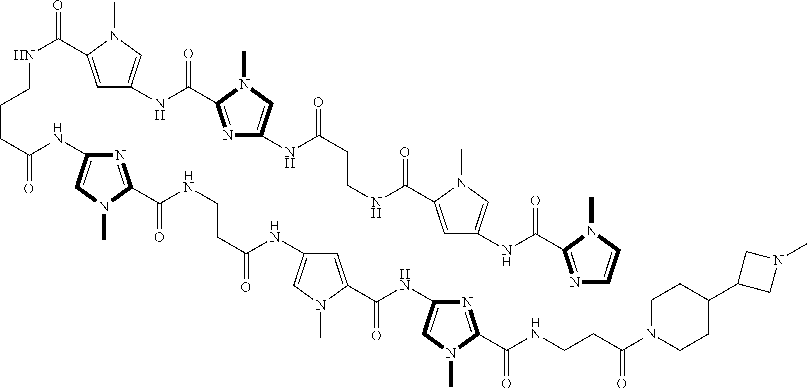 |
| 204 | 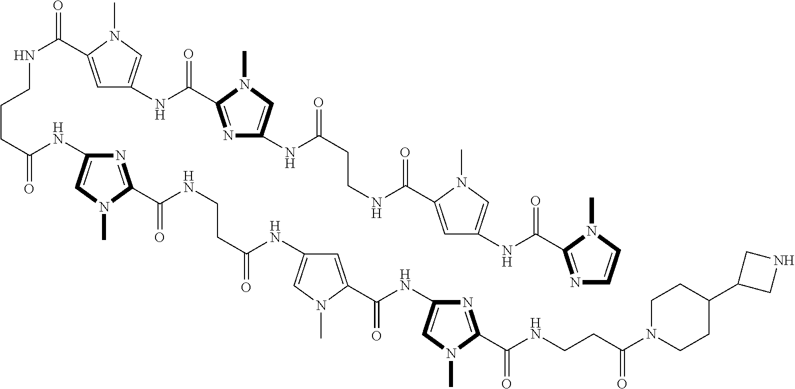 |
| 205 | 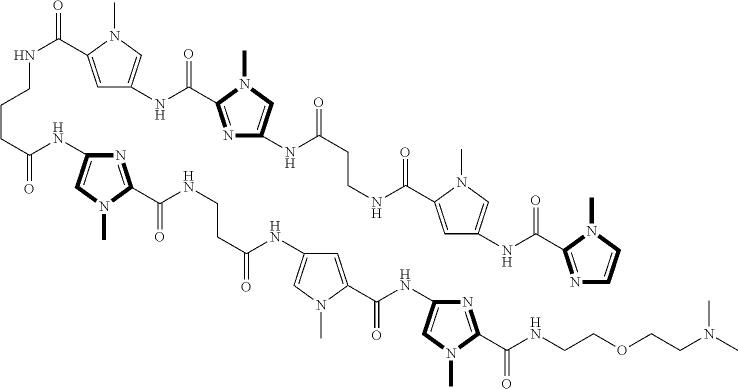 |
| 206 | 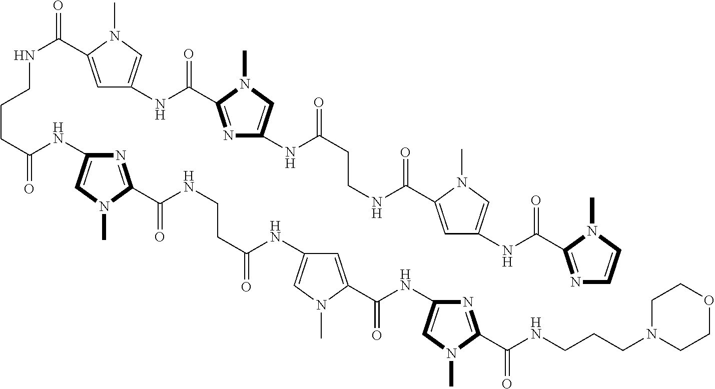 |
| 207 | 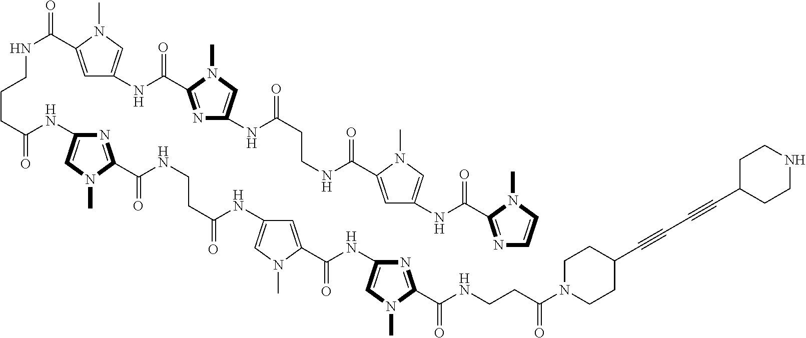 |
| 208 | 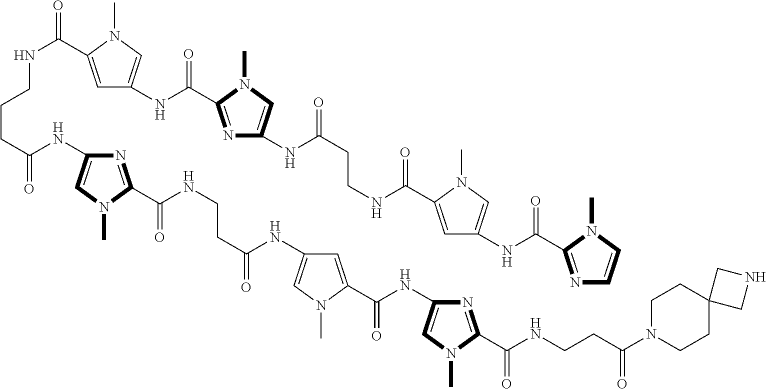 |
| 209 | 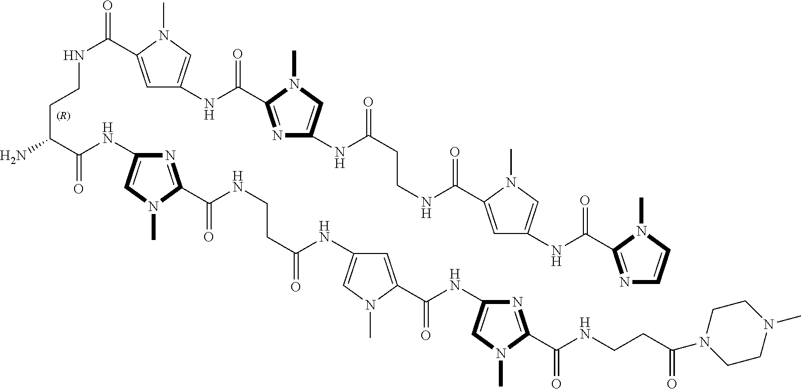 |
| 210 | 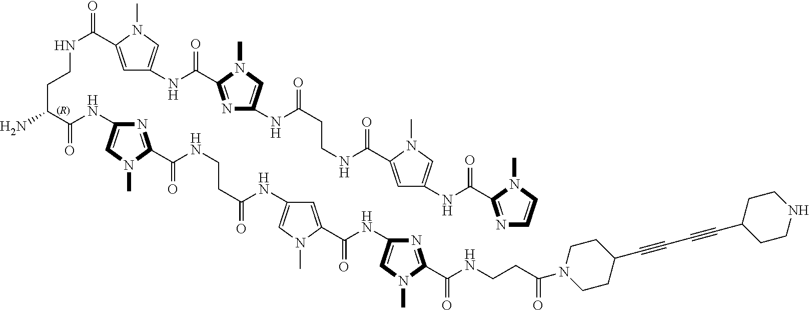 |
| 211 | 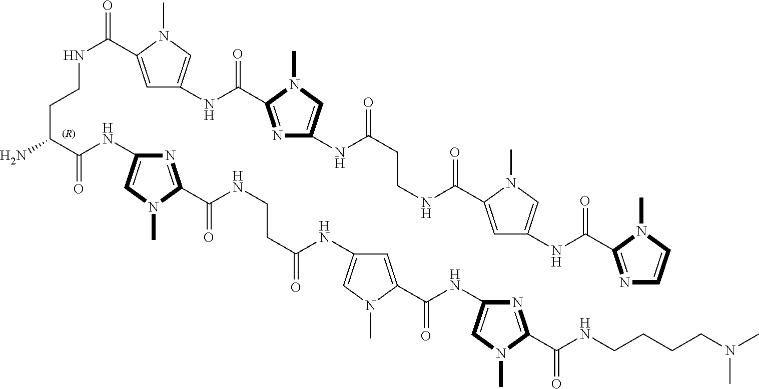 |
| 212 | 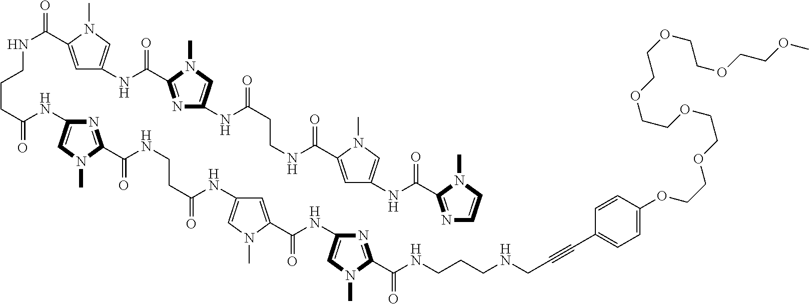 |
| 213 | 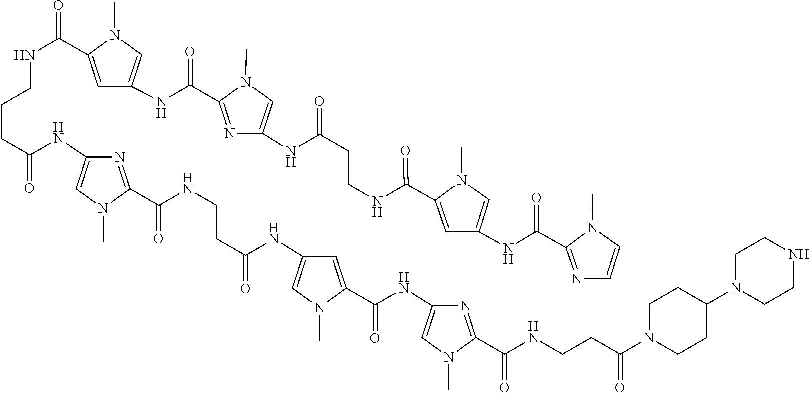 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(N5CCNCC5)CC4)n3)n(C)c2)n1] |
| 214 | 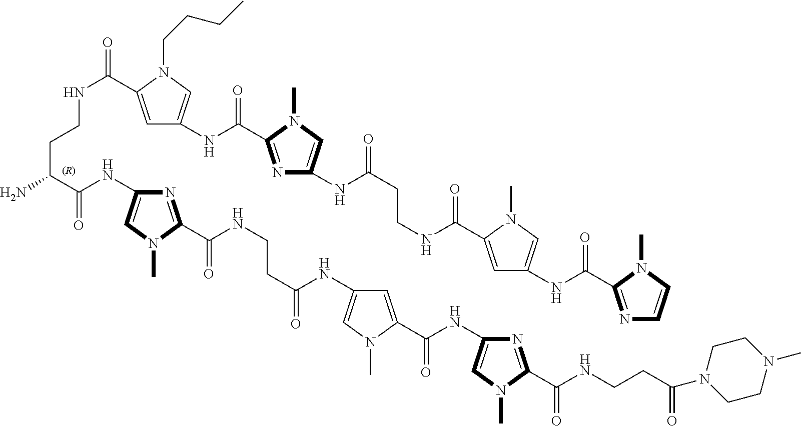 |

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Determination of Compound Solubility in Water

To determine solubility of candidate compounds, solubility of the compounds was determined at a concentration of 10 mg/ml using in situ salt formation with aqueous HCl. First, 0.5 ml of sterile water was added to 10 mg of dry compound, vortexed, and briefly centrifuged. Next, 0.5 equivalents of 0.025 M HCl were added to the mixture followed by a volume of water that totaled 1 ml. The mixture was stirred continuously for 0.5 to 24 h. If clear solution was obtained, the pH value was determined using a microelectrode. If the solution was not clear, additional water and/or HCl was added, along with continuous stirring and vortexing until it was determined that the compound had completely dissolved or was refractory to dissolution. The minimum concentration tested was 1 mg/ml. pH values were assessed at the end of the experiment. Experimental results are shown in Table 2.

TABLE 2
Compound Solubility and pH

| | Final Concentration | | |
|---|---|---|---|
| # | (mg/ml) | Result | pH |
| 207 | 10 | Clear solution | |
| 201 | 10 | Clear solution | 6.04 |
| 208 | 3.53 | Clear solution | 2.75→6.07, aq. NaOH |
| 202 | 4.03 | Clear solution | 3.10→6.24, aq. NaOH |
| 204 | 3.78 | Clear solution | 2.32→6.73, aq. NaOH |
| 203 | 3.18 | Clear solution | 8.04 |
| 206 | 1.00 | Clear solution | 4.96 |
| 205 | 1.00 | Clear solution | 7.62 |
| 209 | 10 | Clear solution | 2.21 |
| 210 | 10 | Clear solution | 2.14 |
| 211 | 10 | Clear solution | 2.14 |
| 212 | 10 | Clear solution | 1.97 |
| 213 | 3.21 | Clear solution | 6.64 |
| 214 | 10 | Clear solution | 2.16 |

### Example 2: Determination of Compound Activity Using In Vitro Foci Assay

Immortalized Fuchs endothelial corneal dystrophy cells (F35T) were seeded in black-sided clear-bottomed 96 well plates at 5000 cells per well 16 hours prior to compound treatment. Compounds were dissolved in DMSO to 1 mM and diluted to a final concentration of 3000 to 1 nM in F35T cell culture media. Diluted compounds were added to the F35T cells and incubated for 48 h.

Following treatment, cells were fixed in 4% paraformaldehyde for 20 min, washed twice with PBS, and permeabilized with 70% ethanol at −20 deg C. for 16 h. Following permeabilization, cells were washed twice with PBS then hydrated with 30% formamide plus 2×SSC buffer for 10 min. Nuclear foci were labeled using fluorescent in situ hybridization with a (CAG)10-Cy3 oligonucleotide. Images were collected and automated image analysis was performed on a BioTEK Cytation 5. Experimental data are provided in Table 3.

TABLE 3
In vitro Foci Assay

| # | IC₅₀ F35T foci 48 h (nM) | Eₘᵢₙ F35T foci 48 h |
|---|---|---|
| 207 | 33 | 0.32 |
| 201 | 167 | 0.32 |
| 208 | 186 | 0.07 |
| 202 | 213 | 0.02 |
| 204 | 518 | 0.15 |
| 203 | 384 | 0.18 |
| 206 | 172 | 0.02 |
| 205 | 65 | 0.14 |
| 209 | 1646 | 0.4088 |
| 210 | 714.8 | 0.3 |
| 211 | 1645 | 0.06904 |
| 212 | 377.6 | 0.172 |
| 213 | 891.7 | 0.1605 |
| 214 | 237.4 | 0.2974 |

### Example 3: Evaluating Eye Penetration of Compounds Formulated in Water

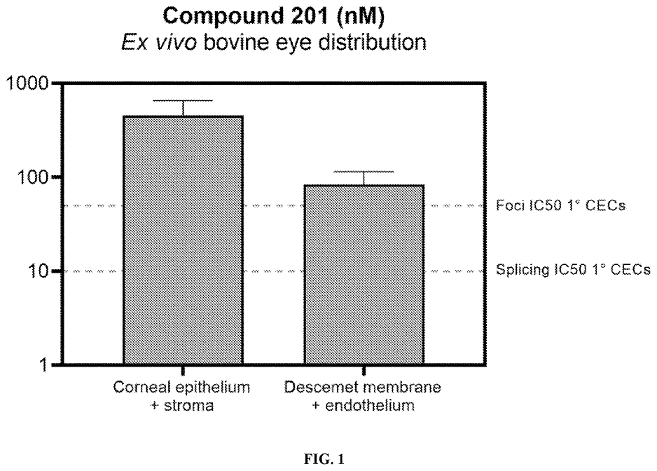

Freshly enucleated bovine eyes (Nebraska Scientific) were treated with compound as described previously (Luschmann et al., “Developing an in situ nanosuspension: a novel approach towards the efficient administration of poorly soluble drugs at the anterior eye”, European Journal of Pharmaceutical Sciences (2013)). Individual eyes were rinsed with 10 mL of sterile 0.9% saline then placed in the bottom of a 600 mL glass beaker (Pyrex). A borosilicate glass powder funnel with a 26 mm diameter stem (Eisco Labs) was gently rested on top of the eye with the cornea centered in the cylindrical opening, creating a sealed dosing chamber in which solutions were applied. Eyes were equilibrated in sterile 0.9% saline at room temperature for 20 min then decanted. 500 μL of 0.1 to 0.3% GeneTAC compound was applied to the cornea followed by 167 μL of sterile water to mimic the instillation a 30 μL eye drop into 10 μL of tear fluid. Compound was incubated for 30 min at room temperature after which the funnel was removed. Next, eyes were washed twice with 10 ml of 5.2% aqueous mannitol solution to remove residual compound. Aqueous humor was recovered using a 28-gauge needle and snap frozen in liquid nitrogen. The cornea was then surgically excised, and the Descemet membrane and corneal endothelium (DM/CE) were removed with fine forceps under a dissecting microscope and snap frozen in liquid nitrogen. Compound abundance was quantified in aqueous humor, anterior cornea, and DM/CE using liquid chromatography-mass spectrometry. Data is shown in Table 4 below. Data is also shown in FIG. 1 which shows that compound 201 reaches concentrations in the corneal epithelium and stroma as well as the Descemet membrane and endothelium that are above the IC50 for preventing foci formation as well as above the IC50 for preventing abnormal splicing events.

TABLE 4
Bovine Eye Penetration

| | Descemet Membrane/ | | Cornea | |
|---|---|---|---|---|
| | endothelial cells (nM) | | (nM) | |
| | R1 | R2 | R1 | R2 |
| 0.1% Compound 201 | 46 | BQL | 798 | 50 |
| Compound 213 | BQL | BQL | 50 | 508 |

BQL—below quantifiable limit

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A composition comprising a double stranded deoxyribonucleic acid (DNA) binding moiety that binds to double stranded DNA in a sequence specific manner, wherein the double stranded DNA binding moiety does not bind to a repeat sequence comprising multiple copies of GAA, and wherein the composition is suitable for administration to a human eye.

2. A composition comprising a double stranded DNA binding moiety suitable for treating a genetic disease, wherein the composition is suitable for administration to a human eye.

3. A composition comprising a double stranded DNA binding moiety that binds to a repeat sequence comprising at least four nucleotides, wherein the composition is suitable for administration to a human eye.

4. A composition comprising a double stranded DNA binding moiety that binds to a repeat sequence comprising multiple copies of CTG, TGC, CAG, GGC, TCCAA, GGGGCC, CGG, GCC, GCG, ATTCT, TGGAA, GGCCTG, CCCCGCCCCGCG, or CCTG, wherein the composition is suitable for administration to a human eye.

5. A composition comprising a double stranded DNA binding moiety, wherein the double stranded DNA binding moiety is a polymer that does not comprise nucleotides or amino acids, and wherein the composition is suitable for administration to a human eye.

6. The composition of any one of claims 1 to 5, wherein the double stranded DNA binding moiety binds to a repeat sequence comprising multiple copies of CTG or CAG.

7. The composition of any one of claims 1 to 6, wherein the double stranded DNA binding moiety binds to a minor groove of double stranded DNA.

8. The composition of any one of claims 1 to 7, wherein the double stranded DNA binding moiety binds to a repeat sequence that is adjacent to or within a gene.

9. The composition of claim 8, wherein the double stranded DNA binding moiety modulates expression of the gene.

10. The composition of claim 8 or claim 9, wherein the gene is Transcription factor 4 (TCF4).

11. The composition of any one of claims 1 to 10, wherein the double stranded DNA binding moiety binds non-covalently.

12. The composition of any one of claims 1 to 4 or 6 to 11, wherein the double stranded DNA binding moiety does not comprise a polynucleotide.

13. The composition of any one of claims 1 to 4 or 6 to 12, wherein the double stranded DNA binding moiety does not comprise a polypeptide.

14. The composition of any one of claims 1 to 13, wherein the double stranded DNA binding moiety has a molecular weight of less than about 10 kDa, about 7.5 kDa, about 5 kDa, or about 2.5 kDa.

15. The composition of any one of claims 1 to 14, wherein the double stranded DNA binding moiety comprises a polyamide.

16. The composition of any one of claims 1 to 15, wherein the double stranded DNA binding moiety further comprises a transcriptional modulator binding moiety.

17. The composition of claim 16, wherein the double stranded DNA binding moiety is connected to the transcriptional modulator binding moiety by a linker.

18. The composition of any one of claims 1 to 17, wherein administration of the composition to an eye results in minimal systemic exposure.

19. The composition of any one of claims 1 to 18, further comprising an excipient.

20. The composition of any one of claims 1 to 19, wherein the ophthalmic formulation composition has a pH of about 5 to about 8.

21. The composition of any one of claims 1 to 20, wherein the composition has a viscosity of from about 1 to about 50,000 cps at about 20° C.

22. A method of treating an eye disorder in a human individual in need thereof, the method comprising administering a double stranded deoxyribonucleic acid (DNA) binding moiety that binds to double stranded DNA in a sequence specific manner, wherein the double stranded DNA binding moiety does not bind to a repeat sequence comprising multiple copies of GAA.

23. A method of treating an eye disorder in a human individual in need thereof, the method comprising administering a double stranded DNA binding moiety, wherein the eye disorder is a genetic disease.

24. A method of treating an eye disorder in a human individual in need thereof, the method comprising administering a double stranded DNA binding moiety to an eye of the individual, wherein administration results in minimal systemic exposure to the double stranded DNA binding moiety, and wherein the double stranded DNA binding moiety binds to a repeat sequence comprising multiple copies of CTG, TGC, CAG, GGC, TCCAA, GGGGCC, CGG, GCC, GCG, GCT, GCA, GAC, AAGGG, ATTCT, TGGAA, GGCCTG, CCCCGCCCCGCG, or CCTG.

25. A method of treating an eye disorder in a human individual in need thereof, the method comprising administering a double stranded DNA binding moiety that binds to a repeat sequence comprising at least four nucleotides.

26. A method of treating an eye disorder in a human individual in need thereof, the method comprising administering a double stranded DNA binding moiety, wherein the double stranded DNA binding moiety is a polymer that does not comprise nucleotides or amino acids.

27. The method of any one of claims 22 to 26, wherein the double stranded DNA binding moiety binds to a repeat sequence comprising multiple copies of CTG or CAG.

28. The method of any one of claims 22 to 27, wherein the double stranded DNA binding moiety binds to a minor groove of double stranded DNA.

29. The method of any one of claims 22 to 28, wherein the double stranded DNA binding moiety binds to a repeat sequence adjacent to or within a gene.

30. The method of claim 29, wherein the double stranded DNA binding moiety modulates expression of the gene.

31. The method of claim 29 or claim 30, wherein the gene is Transcription factor 4 (TCF4).

32. The method of any one of claims 22 to 31, wherein the double stranded DNA binding moiety binds non-covalently.

33. The method of any one of claims 22 to 25 or 27 to 32, wherein the double stranded DNA binding moiety does not comprise a polynucleotide.

34. The method of any one of claims 22 to 25 or 27 to 33, wherein the double stranded DNA binding moiety does not comprise a polypeptide.

35. The method of any one of claims 22 to 34, wherein the double stranded DNA binding moiety has a molecular weight of less than about 10 kDa, about 7.5 kDa, about 5 kDa, or about 2.5 kDa.

36. The method of any one of claims 22 to 35, wherein the double stranded DNA binding moiety comprises a polyamide.

37. The method of any one of claims 22 to 36, wherein the double stranded DNA binding moiety further comprises a transcriptional modulator binding moiety.

38. The method of claim 37, wherein the double stranded DNA binding moiety is connected to the transcriptional modulator binding moiety by a linker.

39. The method of any one of claim 22 or 25 to 38, wherein the double stranded DNA binding moiety is formulated in an excipient suitable for administration to the eye.

40. The method of any one of claims 22 to 39, wherein the double stranded DNA binding moiety is in a having a pH of about 5 to about 8.

41. The method of any one of claims 22 to 40, wherein the double stranded DNA binding moiety is in a having a viscosity of from about 1 to about 50,000 cps at about 20° C.
